SEQUENCE LISTING (21 sequences)

SEQ ID NO: 1 - RNA, 13 nt, Homo sapiens
  5′UTR (1)..(13): /note=“The ′Kozak sequence′ refers to a sequence on eukaryotic mRNA having the consensus (gcc)gccRccAUGG (SEQ ID NO 1), where R is a purine (adenine or guanine) three bases upstream of the start codon (AUG)”
gccgccrccaugg

SEQ ID NO: 2 - DNA, 504 bp, Homo sapiens
  CDS (1)..(501): /note=“Nucleotides 58-561 from Homo sapiens leptin (LEP), mRNA”
atgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgctga

SEQ ID NO: 3 - protein, 167 aa, Homo sapiens
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 4 - RNA, 1080 nt, Homo sapiens
  mRNA (1)..(1080): /note=“TEV-hLeptin-01opt-2xhBG-120A mRNA construct”
gggagacgcguguuaaauaacaaaucucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccgccaccaugcacuggggaacccugugcggauuccuguggcuguggcccuaccuguucuaugugcaagccgugcccauccagaaggugcaggacgacaccaagacccugaucaagaccaucgugacccggaucaacgacaucagccacacccagagcguguccagcaagcagaaagugaccggccuggacuucauccccggccugcacccuauccugacccuguccaagauggaccagacccuggccguguaccagcagauccugaccagcaugcccagccggaacgugauccagaucagcaacgaccuggaaaaccugcgggaccugcugcacgugcuggccuucagcaagagcugccaucugccuugggccagcggccuggaaacccuggauucucugggcggagugcuggaagccagcggcuacucuacagaggugguggcccugagcagacugcagggcagccugcaggauaugcuguggcagcuggaucugagccccggcugcuaauagcggaccggcgauagaugaagcucgcuuucuugcuguccaauuucuauuaaagguuccuuuguucccuaaguccaacuacuaaacugggggauauuaugaagggccuugagcaucuggauucugccuaauaaaaaacauuuauuuucauugcagcucgcuuucuugcuguccaauuucuauuaaagguuccuuuguucccuaaguccaacuacuaaacugggggauauuaugaagggccuugagcaucuggauucugccuaauaaaaaacauuuauuuucauugcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 5 - protein, 167 aa, Homo sapiens <s300> /note=“GenBank Acc. No. NP_000221”; identical to SEQ ID NO: 3

SEQ ID NO: 6 - RNA, 1080 nt, Homo sapiens
  mRNA (1)..(1080): /note=“TEV-hLeptin-02opt-2xhBG-120A construct mRNA”
gggagacgcguguuaaauaacaaaucucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccgccaccaugcacuggggcacccuguguggcuuucuguggcuguggcccuaccuguucuacgugcaggccgugcccauccagaaggugcaggacgacaccaagacccugaucaagaccaucgucaccaggaucaacgacaucucccacacccaguccguguccagcaagcaaaaggugaccggacuggacuucauccccggccugcaucccauccugacccugagcaagauggaccagacacuggccguguaccaacagauccugaccagcaugcccagcaggaacgugauccagaucuccaacgaccuggagaaccucagggaccugcugcacgugcuggccuucagcaagagcugccaucugcccugggcuagcggacuggagacccuggacagccugggaggagugcuggaagccagcggcuacagcacagagguggucgcccugagcaggcuccagggcagccugcaggacaugcuguggcagcuggaccugagccccggaugcugaugacggaccggcgauagaugaagcucgcuuucuugcuguccaauuucuauuaaagguuccuuuguucccuaaguccaacuacuaaacugggggauauuaugaagggccuugagcaucuggauucugccuaauaaaaaacauuuauuuucauugcagcucgcuuucuugcuguccaauuucuauuaaagguuccuuuguucccuaaguccaacuacuaaacugggggauauuaugaagggccuugagcaucuggauucugccuaauaaaaaacauuuauuuucauugcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 7 - protein, 167 aa, Homo sapiens <s300> /note=“GenBank Acc. No. NP_000221”; identical to SEQ ID NO: 3

SEQ ID NO: 8 - RNA, 1080 nt, Homo sapiens
  mRNA (1)..(1080): /note=“TEV-hLeptin-03opt-2xhBG-120A mRNA construct”
gggagacgcguguuaaauaacaaaucucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccgccaccaugcauugggggacacugugcggguuccuguggcuguggcccuaucuguucuacgugcaggcugugcccauccagaagguccaggacgacaccaagacacugaucaaaacuauugugaccaggaucaacgauauuagucacacccagucagugagcuccaagcagaaagucacaggacuggacuucauccccggccugcauccuauucugacacugucuaagauggaucagacucuggccguguaccagcagauccugacaucaaugccaagccggaaugucauccagauuuccaacgaccuggagaaucugagagaucugcugcacgugcuggcauuuucuaaaaguugccaucugccaugggcaagcggacuggaaacucuggacucccugggagggguccuggaggcauccggauauucuaccgaaguggucgcucugagccggcugcagggcucacugcaggauaugcuguggcagcuggaucugucucccggaugcuaauaacggaccggcgauagaugaagcucgcuuucuugcuguccaauuucuauuaaagguuccuuuguucccuaaguccaacuacuaaacugggggauauuaugaagggccuugagcaucuggauucugccuaauaaaaaacauuuauuuucauugcagcucgcuuucuugcuguccaauuucuauuaaagguuccuuuguucccuaaguccaacuacuaaacugggggauauuaugaagggccuugagcaucuggauucugccuaauaaaaaacauuuauuuucauugcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 9 - protein, 167 aa, Homo sapiens <s300> /note=“GenBank Acc. No. NP_000221”; identical to SEQ ID NO: 3

SEQ ID NO: 10 - RNA, 1080 nt, Homo sapiens
  mRNA (1)..(1080): /note=“TEV-hLeptin-nonopt-2xhBG-120A mRNA construct”
gggagacgcguguuaaauaacaaaucucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccgccaccaugcauuggggaacccugugcggauucuuguggcuuuggcccuaucuuuucuauguccaagcugugcccauccaaaaaguccaagaugacaccaaaacccucaucaagacaauugucaccaggaucaaugacauuucacacacgcagucagucuccuccaaacagaaagucaccgguuuggacuucauuccugggcuccaccccauccugaccuuauccaagauggaccagacacuggcagucuaccaacagauccucaccaguaugccuuccagaaacgugauccaaauauccaacgaccuggagaaccuccgggaucuucuucacgugcuggccuucucuaagagcugccacuugcccugggccaguggccuggagaccuuggacagccugggggguguccuggaagcuucaggcuacuccacagaggugguggcccugagcaggcugcaggggucucugcaggacaugcuguggcagcuggaccucagcccugggugcugauaacggaccggcgauagaugaagcucgcuuucuugcuguccaauuucuauuaaagguuccuuuguucccuaaguccaacuacuaaacugggggauauuaugaagggccuugagcaucuggauucugccuaauaaaaaacauuuauuuucauugcagcucgcuuucuugcuguccaauuucuauuaaagguuccuuuguucccuaaguccaacuacuaaacugggggauauuaugaagggccuugagcaucuggauucugccuaauaaaaaacauuuauuuucauugcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 11 - protein, 167 aa, Homo sapiens <s300> /note=“GenBank Acc. No. NP_000221”; identical to SEQ ID NO: 3

SEQ ID NO: 12 - RNA, 1128 nt, Mus musculus
  mRNA (1)..(1128): /note=“TEV-mEpo(NcoI)-2xhBG-120A mRNA construct”
gggagacgcguguuaaauaacaaaucucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccgccaccaugggcgugcccgaaagaccuacacuccugcugcugcugucacugcugcugaucccucugggccugccugugcugugugcccccccuagacugaucugcgacagcagagugcuggaacgguacauccuggaagccaaagaggccgagaacgugacgaugggaugugccgagggccccagacugagcgagaacaucaccgugcccgacaccaaagugaacuucuacgccuggaagcggauggaaguggaagaacaggccaucgaaguguggcagggccugagccugcugagcgaggcuauucugcaggcacaggcucugcuggccaacagcagccagccuccugagacacugcagcugcacaucgacaaggccaucagcggccugagaagccugaccucccugcugagggugcugggagcccagaaagaacugaugagccccccugacaccacccccccugcuccucugagaacucugaccguggacaccuucugcaagcuguuccggguguacgccaacuuccugcggggcaagcugaagcuguacaccggcgaagugugcagacggggcgacagaugaagcucgcuuucuugcuguccaauuucuauuaaagguuccuuuguucccuaaguccaacuacuaaacugggggauauuaugaagggccuugagcaucuggauucugccuaauaaaaaacauuuauuuucauugcagcucgcuuucuugcuguccaauuucuauuaaagguuccuuuguucccuaaguccaacuacuaaacugggggauauuaugaagggccuugagcaucuggauucugccuaauaaaaaacauuuauuuucauugcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 13 - protein, 192 aa, Mus musculus <s300> /note=“GenBank Acc. No. NP_031968”
MGVPERPTLLLLLSLLLIPLGLPVLCAPPRLICDSRVLERYILEAKEAENVTMGCAEGPRLSENITVPDTKVNFYAWKRMEVEEQAIEVWQGLSLLSEAILQAQALLANSSQPPETLQLHIDKAISGLRSLTSLLRVLGAQKELMSPPDTTPPAPLRTLTVDTFCKLFRVYANFLRGKLKLYTGEVCRRGDR

SEQ ID NO: 14 - DNA, 4523 bp, Homo sapiens
  misc_feature (1)..(4523): /note=“Entire plasmid DNA construct for human leptin encoding amino acids 1-167 of Protein Accession # NP_000221, sequence codon, optimized”
gatccggaggccggagaattgtaatacgactcactatagggagacgcgtgttaaataacaaatctcaacacaacatatacaaaacaaacgaatctcaagcaatcaagcattctacttctattgcagcaatttaaatcatttcttttaaagcaaaagcaattttctgaaaattttcaccatttacgaacgatagccgccaccatgcactggggaaccctgtgcggattcctgtggctgtggccctacctgttctatgtgcaagccgtgcccatccagaaggtgcaggacgacaccaagaccctgatcaagaccatcgtgacccggatcaacgacatcagccacacccagagcgtgtccagcaagcagaaagtgaccggcctggacttcatccccggcctgcaccctatcctgaccctgtccaagatggaccagaccctggccgtgtaccagcagatcctgaccagcatgcccagccggaacgtgatccagatcagcaacgacctggaaaacctgcgggacctgctgcacgtgctggccttcagcaagagctgccatctgccttgggccagcggcctggaaaccctggattctctgggcggagtgctggaagccagcggctactctacagaggtggtggccctgagcagactgcagggcagcctgcaggatatgctgtggcagctggatctgagccccggctgctaatagcggaccggcgatagatgaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagcaagctttcgatagcgctgttcgtagaaaaaaaagaagtaaataattactacttgccatatagactaaatagctgcgcntaatacatctacactttctannattgacaagtgatacgttgcaaaaggagcaacaccccacagactcgatgactgcgcagtcatacagtgaaattgccctaatgtcttacctctgaaagggctaaacgaaagtagagcactattccgcgtagctatttagtgcgatcttttagaaatatcagcccagagagctgggctgataaatattttatccgacaagacgaattttgctcaaatgagttaaaacgatgctaccactatctgctgcttttacgagatcagcccaccattgcatcatcggacgacgttgctcaagataatcactgcggttataagcgcgacgcacctcatttttgtctacatgagcaagcgctgcttcaatgacatcaggtggaaatccttcctcattgagtgccgtactggcgatagaacgcaagccgtgtgaaacaagtacacctcctaagccagcacgcttgagtgctgcattcactgtttggctattcattggttggttgggcttgatacggctaggaaagataaattctcggccaccactgagaggcttcatcatttccagaatagcaagagccccatcagatagtggaaccgtatggtcccggttcatcttcattcgagctgcaggaattttccattcgctagcattgaaatcgatctcatcccatcgagcctcagcagcttcggcagggcgggtgatggttagaagttgccacatgaacaggcatcttgtggacatgctgatacttgccgtacgcatggtgtgcattagctgcggaagttgatccggccggatgcttggcatgtttttcttttgcggtttctcgaaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctattccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaattcgagctcggtacccggg

SEQ ID NO: 15 - protein, 167 aa, Homo sapiens <s300> /note=“GenBank Acc. No. NP_000221”; identical to SEQ ID NO: 3

SEQ ID NO: 16 - RNA, 1080 nt, Homo sapiens
  mRNA (1)..(1080): /note=“Non-translatable TEV-hLeptin-GAopt(NT)- 2xhBG-120A (codon optimized leptin coding region rendered non-translatable by mutation of ATG codons)”
gggagacgcguguuaaauaacaaaucucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccgccaccgugcacuggggaacccugugcggauuccuguggcuguggcccuaccuguucucugugcaagccgugcccauccagaaggugcaggacgacaccaagacccugaucaagaccaucgugacccggaucaacgacaucagccacacccagagcguguccagcaagcagaaagugaccggccuggacuucauccccggccugcacccuauccugacccuguccaagguggaccagacccuggccguguaccagcagauccugaccagccugcccagccggaacgugauccagaucagcaacgaccuggaaaaccugcgggaccugcugcacgugcuggccuucagcaagagcugccaucugccuugggccagcggccuggaaacccuggauucucugggcggagugcuggaagccagcggcuacucuacagaggugguggcccugagcagacugcagggcagccugcaggaugugcuguggcagcuggaucugagccccggcugcuaauagcggaccggcgauagaugaagcucgcuuucuugcuguccaauuucuauuaaagguuccuuuguucccuaaguccaacuacuaaacugggggauauuaugaagggccuugagcaucuggauucugccuaauaaaaaacauuuauuuucauugcagcucgcuuucuugcuguccaauuucuauuaaagguuccuuuguucccuaaguccaacuacuaaacugggggauauuaugaagggccuugagcaucuggauucugccuaauaaaaaacauuuauuuucauugcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 17 - RNA, 501 nt, Homo sapiens
  variation (1)..(501): /note=“Codon optimized coding sequence of SEQ ID NO 4”
augcacuggggaacccugugcggauuccuguggcuguggcccuaccuguucuaugugcaagccgugcccauccagaaggugcaggacgacaccaagacccugaucaagaccaucgugacccggaucaacgacaucagccacacccagagcguguccagcaagcagaaagugaccggccuggacuucauccccggccugcacccuauccugacccuguccaagauggaccagacccuggccguguaccagcagauccugaccagcaugcccagccggaacgugauccagaucagcaacgaccuggaaaaccugcgggaccugcugcacgugcuggccuucagcaagagcugccaucugccuugggccagcggccuggaaacccuggauucucugggcggagugcuggaagccagcggcuacucuacagaggugguggcccugagcagacugcagggcagccugcaggauaugcuguggcagcuggaucugagccccggcugc

SEQ ID NO: 18 - RNA, 501 nt, Homo sapiens
  variation (1)..(501): /note=“Codon optimized coding sequence of SEQ ID NO 6”
augcacuggggcacccuguguggcuuucuguggcuguggcccuaccuguucuacgugcaggccgugcccauccagaaggugcaggacgacaccaagacccugaucaagaccaucgucaccaggaucaacgacaucucccacacccaguccguguccagcaagcaaaaggugaccggacuggacuucauccccggccugcaucccauccugacccugagcaagauggaccagacacuggccguguaccaacagauccugaccagcaugcccagcaggaacgugauccagaucuccaacgaccuggagaaccucagggaccugcugcacgugcuggccuucagcaagagcugccaucugcccugggcuagcggacuggagacccuggacagccugggaggagugcuggaagccagcggcuacagcacagagguggucgcccugagcaggcuccagggcagccugcaggacaugcuguggcagcuggaccugagccccggaugc

SEQ ID NO: 19 - RNA, 501 nt, Homo sapiens
  variation (1)..(501): /note=“Codon optimized coding sequence of SEQ ID NO 8”
augcauugggggacacugugcggguuccuguggcuguggcccuaucuguucuacgugcaggcugugcccauccagaagguccaggacgacaccaagacacugaucaaaacuauugugaccaggaucaacgauauuagucacacccagucagugagcuccaagcagaaagucacaggacuggacuucauccccggccugcauccuauucugacacugucuaagauggaucagacucuggccguguaccagcagauccugacaucaaugccaagccggaaugucauccagauuuccaacgaccuggagaaucugagagaucugcugcacgugcuggcauuuucuaaaaguugccaucugccaugggcaagcggacuggaaacucuggacucccugggagggguccuggaggcauccggauauucuaccgaaguggucgcucugagccggcugcagggcucacugcaggauaugcuguggcagcuggaucugucucccggaugc

SEQ ID NO: 20 - RNA, 501 nt, Homo sapiens
  variation (1)..(501): /note=“Coding sequence of SEQ ID NO 10”
augcauuggggaacccugugcggauucuuguggcuuuggcccuaucuuuucuauguccaagcugugcccauccaaaaaguccaagaugacaccaaaacccucaucaagacaauugucaccaggaucaaugacauuucacacacgcagucagucuccuccaaacagaaagucaccgguuuggacuucauuccugggcuccaccccauccugaccuuauccaagauggaccagacacuggcagucuaccaacagauccucaccaguaugccuuccagaaacgugauccaaauauccaacgaccuggagaaccuccgggaucuucuucacgugcuggccuucucuaagagcugccacuugcccugggccaguggccuggagaccuuggacagccugggggguguccuggaagcuucaggcuacuccacagaggugguggcccugagcaggcugcaggggucucugcaggacaugcuguggcagcuggaccucagcccugggugc

SEQ ID NO: 21 - RNA, 1761 nt, Homo sapiens
  mRNA (1)..(1761): /note=“Non-translatable TEV-hIgGH1-59-hLeptin- GAopt(NT)- 2xhBG-120A (codon optimized N-terminal Fc-Leptin fusion coding region rendered non-translatable by mutation of ATG codons)”
gggagacgcguguuaaauaacaaaucucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagccgccacccuggacuggaucuggcgcauccuguuucucgugggagccgccacaggcgcccacagcgauaagacccacaccuguccucccugcccugccccugaagcugcuggcggaccuagcguguuccuguuccccccaaagcccaaggacacccuggugaucagccggacccccgaagugaccugcguggugguggcugugucccacgaggacccugaagugaaguucaauugguacguggacggcguggaagugcacaacgccaagaccaagcccagagaggaacaguacaacagcaccuaccgggugguguccgugcugaccgugcugcaccaggacuggcugaacggcaaagaguacaagugcaagguguccaacaaggcccugccagcccccaucgagaaaaccaucagcaaggccaagggccagccccgcgaaccccagguguacacacugcccccuagccgggaagaggugaccaagaaccaggugucccugaccugucucgugaagggcuucuaccccuccgauaucgccguggaaugggagagcaacggccagcccgagaacaacuacaagaccacccccccugugcuggacagcgacggcucauucuuccuguacagcaagcugacaguggacaagagccgguggcagcagggcaacguguucagcugcagcgugcugcacgaggcccugcacaaccacuacacccagaagucccugagccugagcccuggcaagggcagcgugccaauccagaaggugcaggacgacaccaagacccugaucaagaccaucgugacccggaucaacgacaucagccacacccagagcguguccagcaagcagaaagugaccggccuggacuucauccccggccugcacccuauccugacccuguccaagguggaccagacccuggccguguaccagcagauccugaccagccugcccagccggaacgugauccagaucagcaacgaccuggaaaaccugcgggaccugcugcacgugcuggccuucagcaagagcugccaucugccuugggccagcggccuggaaacccuggauucucugggcggagugcuggaagccagcggcuacucuacagaggugguggcccugagcagacugcagggcagccugcaggaugugcuguggcagcuggaucugagccccggcuguuaauagcggaccggcgauagaugaagcucgcuuucuugcuguccaauuucuauuaaagguuccuuuguucccuaaguccaacuacuaaacugggggauauuaugaagggccuugagcaucuggauucugccuaauaaaaaacauuuauuuucauugcagcucgcuuucuugcuguccaauuucuauuaaagguuccuuuguucccuaaguccaacuacuaaacugggggauauuaugaagggccuugagcaucuggauucugccuaauaaaaaacauuuauuuucauugcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2016/0367638 A1
(43) Pub. Date: Dec. 22, 2016

(54) LEPTIN mRNA COMPOSITIONS AND FORMULATIONS

(71) Applicants: Crystal BYERS, Cambridge, MA (US); Shari Lynn CAPLAN, Cambridge, MA (US); Gabriel Grant GAMBER, Cambridge, MA (US); Seung HAHM, Cambridge, MA (US); Kurt Alex HELDWEIN, Cambridge, MA (US); Igor SPLAWSKI, Cambridge, MA (US); Thomas ZABAWA, Cambridge, MA (US); Frédéric ZECRI, Cambridge, MA (US)

(72) Inventors: Crystal BYERS, Cambridge, MA (US); Shari Lynn CAPLAN, Cambridge, MA (US); Gabriel GAMBERS, Cambridge, MA (US); Seung HAHM, Cambridge, MA (US); Kurt Alex HELDWEIN, Cambridge, MA (US); Igor SPLAWSKI, Cambridge, MA (US); Thomas ZABAWA, Cambridge, MA (US); Frédéric ZECRI, Cambridge, MA (US)

(21) Appl. No.: 15/104,843

(22) PCT Filed: Dec. 17, 2014
(86) PCT No.: PCT/US2014/070896

§ 371 Date: Jun. 15, 2016

Related U.S. Application Data
(60) Provisional application No. 61/918,161, filed on Dec. 19, 2013.
(60) Provisional application No. 61/933,964, filed on Jan. 31, 2014.

(51) Int. Cl. A61K 38/22; A61K 9/00; A61K 9/16

(52) U.S. Cl. CPC A61K 38/2264; A61K 9/1617; A61K 9/1641; A61K 9/0019; A61K 48/00

(57) ABSTRACT

A formulation comprising a modified synthetic messenger RNA and a delivery agent. The modified synthetic messenger RNA encodes a leptin protein, is non-replicating and is translation-ready. The formulation can be delivered to a subject with congenital leptin deficiency, lipodystrophy or other condition where circulating leptin level is low.

### FIELD OF THE INVENTION

The invention relates generally to polynucleotide bio-affecting or body eating compositions, and specifically to the treatment of a subject with congenital leptin deficiency, lipodystrophy or other condition where circulating leptin level is low, by administering to the subject a modified, synthetic, non-replicating messenger ribonucleic acid (mRNA) encoding a human leptin protein.

### BACKGROUND OF THE INVENTION

Leptin is a hormone that is produced and secreted by white adipose tissue into the circulatory system of an individual. Circulating leptin enters the individual's brain, where it binds to leptin receptors to regulate appetite (feeling of satiety), energy metabolism and neuroendocrine function by activating several signal transduction cascades.

Congenital leptin deficiencies are caused by mutations in the leptin gene. Individuals with congenital leptin deficiency eat voraciously and become morbidly obese, suffering the consequences of obesity, including hypogonadism and diabetes. Individuals with leptin mutations or with monogenic mutations that cause generalized lipodystrophy (near complete absence of adipose tissue) also have inadequate leptin levels. These individuals with lipodystrophy and low leptin levels are lean, but develop hyperphagia, severe lipid abnormalities and diabetes.

Treatment with recombinant leptin protein reverses these effects. Licinio J et al. (2004) Proc. Natl. Acad. Sci. USA 101(13):4531-6, showed weight loss in leptin-deficient obese adults following a leptin protein therapy. Patients were treated with metreleptin (a recombinant leptin protein) subcutaneously for 18 months, which resulted in increased physical activity, resolution of both type 2 diabetes and hypogonadism, and a reduction in body mass index. In patients with generalized lipodystrophy, Ebihara K et al. (2007) J. Clin. Endocrinol. Metab. 92 532-541 showed that a 40-month treatment with metreleptin by twice-daily injection improved fasting glucose and triglyceride levels, beginning within 1 week. The leptin-replacement therapy reduced insulin resistance and augmented insulin secretion. The metreleptin-therapy was beneficial for both diabetic and lipodystrophic complications. Chan J L et al. (2011) Endocr. Pract. 17(6):922-932, showed that metreleptin therapy normalized the metabolic abnormalities in lipodystrophic patients. In particular, Chan et al. (2011) showed that metreleptin therapy reduced hemoglobin A1c and triglyceride levels throughout a 3-year treatment period.

However, leptin protein is difficult to produce and has a short half-life, requiring once or twice daily subcutaneous (SC) dosing. Thus, there is a need in the art for improving the patient standard of care by decreasing the leptin dosing regimen from twice daily to once every several days, or even less frequently.

### SUMMARY OF THE INVENTION

The invention provides a formulation that is useful for correcting leptin deficiency in a subject. The formulation can advantageously be administered to a subject once every several days with minimal immune activation and with controlled expression of leptin protein in vivo.

One component of the formulation is a modified synthetic leptin messenger ribonucleic acid (mRNA). The modification of the mRNA provides improved mRNA stability and decreased immunogenicity. The synthesis of the modified synthetic leptin mRNA can be by any of several methods, including in vitro transcription. In one embodiment, leptin mRNA is modified during its synthesis by the substitution of the uridines in the leptin mRNA with pseudouridine (ψ) during an in vitro transcription of the leptin mRNA. In a specific embodiment, all of the uridines in the leptin mRNA are substituted with pseudouridine.

In one embodiment, the modified synthetic leptin has a coding sequence that is found in nature. In an alternative embodiment, the modified synthetic leptin has a coding sequence that is codon optimized. In one embodiment, the modified synthetic leptin has a coding sequence that encodes a protein that is functionally equivalent to native human leptin protein.

Another component of the formulation is a delivery agent. In one embodiment, the delivery agent is a lipid nanoparticle. In one embodiment, the cationic lipid nanoparticle comprises (i) a cationic lipid for encapsulation and for endosomal escape, (ii) a neutral lipid, for stabilization, (iii) a helper lipid, also for stabilization, and (iv) a stealth lipid, which prevents aggregation. In a more specific embodiment, the cationic lipid can be Cationic Lipid A, Cationic Lipid B, Cationic Lipid C or Cationic Lipid D; the helper lipid is cholesterol; and the stealth lipid is a polyethylene glycol (PEG) lipid (“lipidated PEG”). In another embodiment, improved lipid nanoparticles are used as delivery agents to provide improved expression of leptin protein in an individual's cells and increased leptin in an individual's circulation. In another embodiment, the helper lipid is 1,2-distearoyl-sn-glycero-3-phosphocholine (DSPC). In yet another embodiment, the stealth lipid is S024 (described further herein). In another embodiment, the molar ratio of cationic lipid to neutral lipid is between 3:1 and 8:1.

The invention also provides a method of administering the modified synthetic leptin mRNA formulations to a subject with a leptin deficiency. The subjects may have condition such as congenital and acquired generalized lipodystrophy (in which there are very low leptin levels, acquired HIV lipodystrophy (in which there are low leptin levels), hypothalamic amenorrhea or obesity (in particular, those with decreased leptin levels). The modified synthetic leptin mRNA formulation can be administered to a subject in vivo or to an organ or tissue ex vivo to induce exogenous expression of the leptin proteins in an organ or in adipose tissue. Accordingly, the modified synthetic leptin mRNA formulation of the invention can be used for purposes similar to gene therapy, with minimal cancer risk, because the mRNA is incapable of being reverse transcribed in mammalian cells to generate DNA copies that could pose a chromosome insertion risk.

In one embodiment, the modified synthetic leptin mRNA formulation is administered to the individual intravenously. In another embodiment, the modified synthetic leptin mRNA formulation is administered subcutaneously. In yet another embodiment, the modified synthetic leptin mRNA formulation is first administered intravenously, and then administered subcutaneously. The modified synthetic leptin mRNA formulation can be administered in repeat dosages. In one embodiment, the modified synthetic leptin mRNA formulation of the invention is delivered at a dosage of at least 0.2 mg leptin mRNA/kg of the subject's body weight. In another embodiment, the modified synthetic leptin mRNA formulation of the invention is delivered at a dosage of at least 0.6 mg leptin mRNA/kg of the subject's body weight.

The invention has several advantages. One of the advantages is that the formulation can be dosed to the individual less frequently than the dosing required for the administration of leptin protein, which is once or twice daily subcutaneous dosing. In one embodiment, the administration of the modified synthetic leptin mRNA formulation can be once every three days. In another embodiment, the administration of the modified synthetic leptin mRNA formulation can be once a week. In one embodiment, the modified synthetic leptin mRNA is packaged in a lipid nanoparticle complex that provides a low immunogenicity level to the individual. In another embodiment, the modified synthetic leptin mRNA is advantageously packaged in a delivery agent that is biodegradable.

One of the advantages is that administration of the formulation to a subject delivers sufficient modified synthetic leptin mRNA to result in pharmaceutically active levels of leptin in vivo. As described herein, an EC₅₀ concentration value of the modified synthetic leptin mRNA of the invention has been determined as 1.4 ng/mL (85 pM) in plasma for the suppression of food intake. In one embodiment, the modified synthetic leptin mRNA formulation of the invention is administered to a subject so that the administration results in a plasma concentration of at least 2.8 ng/mL leptin protein, which value is twice the EC₅₀ of human leptin protein concentration for decreasing body weight in leptin-deficient ob/ob mice. In another embodiment, delivery of the modified synthetic leptin mRNA formulation results in a plasma concentration of 1.4 ng/mL leptin protein. In yet another embodiment, delivery of the modified synthetic leptin mRNA formulation results in a plasma concentration of 185 ng/mL. In another embodiment, delivery of the modified synthetic leptin mRNA formulation results in a plasma leptin protein concentration of 1300 ng/mL. In one embodiment, the modified synthetic leptin mRNA formulation of the invention is administered to a subject at an amount sufficient for a plasma leptin protein concentration of at least 10 ng/mL above the subject's baseline leptin protein concentration before the administration.

The invention provides several measurable benefits to the subject to whom the modified synthetic leptin mRNA formulation of the invention is administered. Delivery of the modified synthetic leptin mRNA formulation of the invention induces dose dependent decrease in food intake and body weight. Delivery of the modified synthetic leptin mRNA formulation of the invention ameliorates obesity and diabetes. In one embodiment, the administration results in a decrease of plasma concentration of glucose by at least 30%. In one embodiment, the administration of the leptin mRNA of the invention results in a decrease of plasma concentration of triglycerides by at least 40%.

The administration of the modified synthetic leptin mRNA of the invention to lean subjects results in a lower level of leptin protein in the circulation than administration of the modified synthetic leptin mRNA of the invention to obese subjects. These results show that the modified synthetic leptin mRNA formulation of the invention has advantages for the treatment of obese subjects.

### BRIEF DESCRIPTION OF THE DRAWINGS

FIG. 1 shows the chemical structures of cationic lipids (Cationic Lipid A, Cationic Lipid B, Cationic Lipid C and Cationic Lipid D) used for formulating the in vivo delivery of modified synthetic human leptin mRNA.

FIG. 2 is a set of graphs showing that the administration of human leptin mRNA formulated in a lipid nanoparticle with Cationic Lipid A specifically and transiently reverses the obese and hyperphagic phenotype of leptin deficient ob/ob mice because of a transient restoration of leptin protein expression. Phosphate buffered saline (PBS) or mRNAs encoding either human leptin (hLeptin or hLep) (SEQ ID NO: 4) or mouse erythropoietin (mEPO) (SEQ ID NO: 12) formulated with Cationic Lipid A were administered intravenously to leptin-deficient ob/ob mice at 0.2 mg of mRNA per kilogram of mouse body weight (mg/kg, mpk). Taken together, FIG. 2A-C shows that, compared to PBS and mEPO mRNA controls, human leptin mRNA caused a decrease in body weight (FIG. 2A) that correlates with a decrease in food intake (FIG. 2B) and expression of human leptin protein (FIG. 2C). FIG. 2A is a set of line graphs showing the specific effect of human leptin mRNA (hLeptin) packaged in Cationic Lipid A and administered intravenously on body weight. See EXAMPLE 9 for further details. FIG. 2B is a set of bar graphs showing the specific effect of human leptin mRNA (hLep) packaged in Cationic Lipid A and administered intravenously on food intake. See EXAMPLE 9 for further details. FIG. 2C is a line graph showing the expression levels of human leptin protein (Leptin) after administration of human leptin mRNA packaged in Cationic Lipid A and administered intravenously. See EXAMPLE 10 for further details. mEPO protein expression was high in mice receiving mEPO mRNA (54,582 pg/mL at 6 hours), confirming mEPO mRNA delivery.

FIG. 3 is a set of graphs showing that administration of human leptin mRNA formulated in a lipid nanoparticle with Cationic Lipid B specifically and transiently reverses the obese and hyperphagic phenotype of leptin-deficient ob/ob mice because of a transient restoration of leptin protein expression. Phosphate-buffered saline (PBS) or human leptin mRNA (hLeptin) (SEQ ID NO: 4) or mouse erythropoietin (mEPO) (SEQ ID NO: 12) formulated with Cationic Lipid B were administered intravenously to leptin-deficient ob/ob mice. Human leptin mRNA was administered at 0.02, 0.06, and 0.2 milligrams per kilogram of body weight (mg/kg; mpk). In a parallel study, mouse erythropoietin mRNA was administered at 0.2 mpk. The results show that, compared to PBS, human leptin mRNA caused decreases in body weight (FIG. 3A) that correlate with decreases in food intake (FIG. 3B) and expression of human leptin protein (FIG. 3C). FIG. 3A is a set of line graphs showing the specific effect of different amounts of human leptin mRNA (hLeptin) packaged in Cationic Lipid B and administered intravenously on body weight. See EXAMPLE 11 for further details. FIG. 3B is a set of bar graphs showing the specific effect of different amounts of human leptin mRNA (hLeptin) packaged in Cationic Lipid B and administered intravenously on food intake (FIG. 3B). See EXAMPLE 11 for further details. FIG. 3C is a bar graph showing the expression levels of human leptin protein (Leptin) after administration of 0.6 mpk human leptin mRNA packaged in Cationic Lipid B and administered intravenously. See EXAMPLE 12 for further details. FIG. 3D is a line graph showing that the administration of mouse erythropoietin mRNA (mEPO) does not cause a decrease in body weight in mice. The efficacy of the administration of human leptin mRNA on decreasing body weight is specific. See EXAMPLE 15 for further details. mEPO protein expression was high in mice receiving mEPO mRNA (892,633 pg/mL at 6 hours), confirming mEPO mRNA delivery.

FIG. 4 is a set of graphs showing that administration of human leptin mRNA formulated with Cationic Lipid C specifically and transiently reverses the obese and hyperphagic phenotype of leptin-deficient ob/ob mice because of a transient restoration of leptin protein expression. PBS or human leptin mRNA (SEQ ID NO: 4) or mouse erythropoietin (mEPO) (SEQ ID NO: 12) formulated in a lipid nanoparticle with Cationic Lipid C were administered intravenously to leptin-deficient ob/ob mice. Human leptin mRNA was administered at 0.02, 0.06, and 0.2 mpk. In a parallel study mEPO mRNA was administered at 0.2 mpk. The results show that, compared to PBS, human leptin mRNA caused decreases in body weight (FIG. 4A) that correlate with decreases in food intake (FIG. 4B) and expression of human leptin protein (FIG. 4C). FIG. 4A is a set of line graphs showing the specific effect of human leptin mRNA packaged in Cationic Lipid C on body weight. See EXAMPLE 13 for further details. FIG. 4B is a set of bar graphs showing the specific effect of human leptin mRNA packaged in Cationic Lipid C on food intake. See EXAMPLE 13 for further details. FIG. 4C is a bar graph showing the expression levels of human leptin protein after administration of human leptin mRNA packaged in Cationic Lipid C. See EXAMPLE 14 for further details. FIG. 4D is a line graph showing that the administration of mouse erythropoietin mRNA (mEPO) does not cause a decrease in body weight in mice. The efficacy of the administration of human leptin mRNA on decreasing body weight is specific. See EXAMPLE 15 for further details. mEPO protein expression was high in mice that received mEPO mRNA (158,865 pg/mL at 6 hours), confirming mEPO mRNA delivery.

FIG. 5 is a line graph showing the plasma leptin protein levels following intravenous (IV) vs. subcutaneous (SC) delivery of leptin mRNA formulated in Cationic Lipid A to lean mice. See EXAMPLE 7 (IV) and EXAMPLE 8 (SC) for further details. The efficacious concentration is equivalent to the EC₅₀ for suppression of food intake in leptin-deficient ob/ob mice, as shown in EXAMPLE 1.

FIG. 6 is a set of pictures showing the encapsulation setup for Improvement Process A. See EXAMPLE 34 for further details. See also EXAMPLE 5 for additional information about Improvement Process A.

FIG. 7 is a set of pictures showing the encapsulation setup for Improvement Process B. FIG. 7A shows the encapsulation setup for Improvement Process B. FIG. 7B shows the setup of syringes on two syringe pumps for Process B. FIG. 7C shows the second dilution setup for Improvement Process B. See EXAMPLE 35 for further details.

### DETAILED DESCRIPTION OF THE INVENTION

### Introduction

Disclosed herein are compositions and methods for the production of a modified synthetic leptin mRNA. The compositions and methods of the invention do not include exogenous DNA or viral vector-based methods for the expression of leptin proteins, and thus do not cause permanent modification of the genome or have the potential for unintended mutagenic effects. The compositions, formulations and methods of the invention are based upon the direct introduction of in vitro synthesized RNAs into a cell, which, when translated in vitro or in vivo, provide desired leptin proteins.

One of the objects of modifying the synthetic leptin mRNA of the invention is to reduce immunogenicity. Higher eukaryotic cells have cellular defenses against foreign, “non-self,” RNA. These defenses cause the global inhibition of cellular protein synthesis, resulting in cellular toxicity. The cellular defenses normally recognize in vitro synthesized RNAs as foreign, and induce this cellular innate immune response. In the methods described herein, the effect of the cellular innate immune response is mitigated by using synthetic RNAs that are modified in a manner that avoids or reduces the response. Avoidance or reduction of the innate immune response permit sustained expression from exogenously introduced RNA. In one aspect, sustained expression is achieved by repeated introduction of a modified synthetic leptin mRNA of the invention.

U.S. Pat. No. 8,278,036 to Kariko et al. discloses mRNA molecules with uridine replaced by pseudouridine (ψ), methods of synthesizing the same, and methods for the delivery of therapeutic proteins in vivo. The patent discloses many mRNAs that can be made by the disclosed methods. See also, Kariko K et al. (2007) Current Opinion in Drug Discovery and Development 10(5): 523-532; Kariko K et al. (2008) Molecular Therapy 16(11), 1833-1840 and Anderson B R et al. (2010) Nucleic Acids Res. 38(17): 5884-5892.

The invention provides in vivo potency data for leptin mRNA packaged in a novel lipid nanoparticle complex and testing data in vivo for leptin protein expression following intravenous and subcutaneous delivery.

### DEFINITIONS

“Added co-transcriptionally” means the addition of a feature, e.g., a 5′ diguanosine cap or other modified nucleoside or nucleotide, to a modified synthetic leptin mRNA of the invention during transcription of the RNA molecule (i.e., the modified RNA is not fully transcribed prior to the addition of the 5′ cap).

“Biodegradable” means that the material breaks down in the body of a subject and loses its chemical identity. A biodegradable lipid is a lipid that is rapidly cleared in vivo, as compared to most lipids. The biodegradable lipid moiety disappears rapidly following the peak of tissue exposure after administration in vivo. The clearance can be measured by pharmacokinetically as half-time in the liver. See, Intl. Pat. Appl. No. WO 2011/153493. A biodegradable polymer can be a polymer of the type used in medical devices to avoid a second operation to remove them or to gradually release a drug.

A “coding region (CDS)” or “coding sequence” is the part of a messenger RNA (mRNA) that codes for a polypeptide, such as a protein. A coding region typically begins with an AUG codon and terminates with one or more stop codons. Several exemplary codon regions are described herein. Methods for determining other coding regions are also described herein. A eukaryotic messenger RNA contains other parts that are useful for the translation of information in a coding region to a polypeptide, but which are not themselves coding regions. Such other parts of a messenger RNA are described further herein. As used herein, the term “open reading frame (ORF)” is also used to describe a coding region or coding sequence.

“Contacting” a cell means contacting a cell in vivo or in vitro with a modified synthetic leptin mRNA of the invention or a formulation thereof. Where such a cell is in vivo, contacting the cell with a modified synthetic leptin mRNA of the invention includes administering the modified synthetic leptin mRNA of the invention in a formulation to a subject by an appropriate administration route, such that the compound contacts the cell in vivo.

“Conserved” nucleotides or amino acids are residues of a polynucleotide sequence or polypeptide sequence, respectively, which occurs unaltered in the same position of two or more sequences being compared. Nucleotides or amino acids that are relatively conserved are those that are conserved amongst more related sequences than nucleotides or amino acids appearing elsewhere in the sequences. Two or more sequences are “completely conserved” if they are 100% identical to one another. In some embodiments, two or more sequences are “highly conserved” if they are at least 90% identical, to one another. In some embodiments, two or more bases are “conserved” if they are identical, to one another. Conservation of sequence may apply to the entire length of an oligonucleotide or polypeptide or may apply to a portion, region or feature thereof.

“Delivery” means the act or manner of delivering a compound, substance, entity, moiety, cargo or payload. A “delivery agent” is any substance which facilitates, at least in part, the in vivo delivery of a nucleic acid molecule to cells.

A “deletion” is a mutation in which a section of DNA is lost or deleted.

A “detectable label” means one or more markers, signals, or moieties which are attached, incorporated or associated with another entity (such as RNA or protein) that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, strepavidin and haptens, quantum dots, and the like. Detectable labels may be located at any position in the RNAs or proteins disclosed herein.

A “dosing regimen” is a schedule of administration or physician determined regimen of treatment, prophylaxis, or palliative care.

An “exogenous” nucleic acid is a nucleic acid (e.g., a modified synthetic leptin mRNA of the invention) that has been introduced by a process involving human intervention into a biological system such as a cell or organism in which it is not normally found, or in which it is found in lower amounts. A factor (e.g., a modified synthetic leptin mRNA of the invention) is exogenous if it is introduced into an immediate precursor cell or a progeny cell that inherits the substance. By contrast, an “endogenous” is a factor or expression product that is native to the biological system or cell (e.g., endogenous expression of a gene).

“Expression” is the cellular processes involved in producing RNA and proteins and as appropriate, secreting proteins, including transcription, translation, folding, modification and processing. “Expression” of a nucleic acid sequence refers to one or more of the following events: (i) production of an RNA template from a DNA sequence (e.g., by transcription); (ii) processing of an RNA transcript (e.g., by splicing, editing, 5′ cap formation, or 3′ end processing); (iii) translation of an RNA into a polypeptide or protein; and (iv) post-translational modification of a polypeptide or protein. “Expression products” or “gene products” include RNA transcribed from a gene and polypeptides obtained by translation of mRNA transcribed from a gene.

A “frameshift” is a mutation caused by insertions or deletions) of a number of nucleotides that is not evenly divisible by three from a DNA sequence. Due to the triplet nature of gene expression by codons, the insertion or deletion can change the reading frame (the grouping of the codons), resulting in a completely different translation from the original. This often generates truncated proteins that result in loss of function.

“Homology” means the overall relatedness between nucleic acid molecules (e.g. DNA molecules or RNA molecules) or between polypeptide molecules. The term “homologous” necessarily refers to a comparison between at least two sequences (polynucleotide or polypeptide sequences).

“Identity” means the overall relatedness between polymeric molecules, e.g., between oligonucleotide molecules (e.g., DNA molecules or RNA molecules) or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described by the National Center for Biotechnology Information (http://www.ncbi.nlm.nih.gov/). For example, the percent identity between two nucleotide sequences can be determined using Clustal 2.0 multiple sequence alignment program. Larkin M A et al. (2007) “Clustal @ and Clustal X version 2.0.” Bioinformatics 23(21): 2947-2948.

“Innate immune response” or “interferon response” means a cellular defense response initiated by a cell in response to recognition of infection by a foreign organism, such as a virus or bacteria or a product of such an organism, e.g., an RNA lacking the modifications characteristic of RNAs produced in the subject cell. The innate immune response protects against viral and bacterial infection by inducing the death of cells that detect exogenous nucleic acids. A description of the “innate immune response” or “interferon response” is provided by U.S. Pat. No. 8,278,036 to Kariko et al.

An “isolated cell” is a cell that has been removed from an organism in which it was originally found, or a descendant of such a cell. Optionally the cell has been cultured in vitro, e.g., in the presence of other cells. Optionally, the cell is later introduced into a second organism or re-introduced into the organism from which it (or the cell or population of cells from which it descended) was isolated.

An “insertion” is a mutation in which extra base pairs are inserted into a place in the DNA.

“Leptin” is a protein of about 16 kDa that is involved with regulating energy intake and expenditure, including appetite and hunger, metabolism, and behavior, in a subject. As used herein, the term “leptin” includes any protein that can function as a leptin protein, as measured by in vivo function or in vitro function, such as by binding functionally to a human leptin receptor (LEPR, CD295). The term “human leptin” includes native human leptin (LEP), which has the sequence of Protein Accession # NP_000221 (SEQ. ID NO: 3) and any variants of human leptin that function as a human leptin protein. Mammalian non-human leptin polypeptides generally bear 67% or greater identity to human leptin. Doyon C et al. (2001) “Molecular Evolution of Leptin.” Gen. and Comp. Endocrinol. 124:188-198; Denver R J et al. (2011) “Evolution of Leptin Structure and Function.” Neuroendocrinol. 94:21-38. The term “leptin” also includes metreleptin, a synthetic analog of human leptin, the use of which is described by Licinio et al. (2004), Ebihara K et al. (2007) and Chan et al. (2011). The term “leptin” also includes any teleost or amphibian leptin orthologs that has the functional characteristics of human leptin. For example, Xenopus leptin activates human leptin receptor expressed on cells. Hen G et al. (2008) “Monitoring leptin activity using the chicken leptin receptor.” J. Endocrinol. 197:325-333.

“Modified” means a changed state or structure of a molecule of the invention. A “modified” mRNA contains ribonucleosides that encompass modifications relative to the standard guanine (G), adenine (A), cytidine (C), and uridine (U) nucleosides. The nonstandard nucleosides can be naturally occurring or non-naturally occurring. RNA can be modified in many ways including chemically, structurally, and functionally, by methods known to one of skill in the art. Such RNA modifications can include, for example, modifications normally introduced post-transcriptionally to mammalian cell mRNA. Moreover, mRNA molecules can be modified by the introduction during transcription of natural and non-natural nucleosides or nucleotides, as described in U.S. Pat. No. 8,278,036 to Kariko et al. and in U.S. Pat. Appl. No. 2013/0102034 to Schrum, U.S. Pat. Appl. No. 2013/0115272 to deFougerolles et al. and U.S. Pat. Appl. No. 2013/0123481 to deFougerolles et al. Modified, as it pertains to the modified synthetic leptin mRNA of the invention may also mean any alteration which is different from the wild type human leptin coding sequence.

A “patient” is a subject who may seek or be in need of treatment, requires treatment, is receiving treatment, will receive treatment, or a subject who is under care by a trained professional for a particular disease or condition.

The phrase “pharmaceutically acceptable” is refers to those compounds, materials, compositions or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salts” are derivatives of the compound of the invention wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts of the parent compound formed, for example, from non-toxic inorganic or organic acids. The pharmaceutically acceptable salts can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Lists of suitable salts are found in Remington's The Science and Practice of Pharmacy, 22^{nd} edition (20012) Allen L V et al. eds., Pharmaceutical Press and Journal of Pharmaceutical Science (1977) 66, 2.

“Primers” are short nucleic acid sequences. Polymerase chain reaction (PCR) primers are typically oligonucleotides of fairly short length (e.g., 8-30 nucleotides) that are used in polymerase chain reactions. PCR primers and hybridization probes can readily be developed and produced by those of skill in the art, using sequence information from the target sequence. See, Sambrook et al. (1989) Molecular Cloning: A Laboratory Manual, Cold Spring Harbor Labs Press.

A “probe” (oligonucleotide probe) is a nucleic acid molecule which typically ranges in size from about 50-100 nucleotides to several hundred nucleotides to several thousand nucleotides in length. Therefore, a probe can be any suitable length for use in an assay described herein, including any length in the range of 50 to several thousand nucleotides, in whole number increments. Such a molecule is typically used to identify a specific nucleic acid sequence in a sample by hybridizing to the specific nucleic acid sequence under stringent hybridization conditions. Hybridization conditions are known in the art. See, e.g., Sambrook et al. (1989) Molecular Cloning: A Laboratory Manual, Cold Spring Harbor Labs Press.

“Reduced cytotoxicity” means the death of less than 50% of the cells in a cell culture repeatedly contacted with a modified synthetic leptin mRNA of the invention e.g., compared to contact with an RNA molecule having the same sequence) but lacking modifications to the RNA. The reduced cytotoxicity can be assessed by measuring apoptosis using e.g., a TUNEL assay. Other useful measures for determining “reduced cytotoxicity” include e.g., flow cytometric and bead based measurements of viability, cell growth or cellularity (measured e.g., microscopically and quantitated by a hemocytometer).

“Repeated administrations” are administrations to a subject a plurality of times (e.g., more than once or at least twice). In some embodiments, the frequency of administration occurs every 24-48 hours or more during a given time period. The frequency can also vary, such that the interval between each dose is different (e.g., first interval 36 hours, second interval 48 hours, third interval 72 hours, etc.).

A “ribonucleic acid (RNA) polynucleotide” is a polymer of ribonucelotides, as is known to those of skill in the biological and chemical arts. Each nucleotide in an RNA molecule contains a ribose sugar, with carbons numbered 1′ through 5′. A base is attached to the 1′ position. In general, the bases are adenine (A), cytosine (C), guanine (G), or uracil (U), although many modifications are known to those of skill in the art. For example, as described herein, an RNA may contain one or more pseudouracil (ψ) base, such that the pseudouridine nucleotides are substituted for uridine nucleotides. Many other RNA modifications are known to those of skill in the art, as described herein. Among the kinds of RNA is “messenger ribonucleic acid (mRNA)”, which acts in nature to convey genetic information from DNA to the ribosome, where they specify the amino acid sequence of the protein products of gene expression by a process known as transcription. Structurally and informationally, messenger RNA encodes the information for a protein in a coding region, as is known to those of skill in the biological arts. See, Sambrook et al. (1989) Molecular Cloning: A Laboratory Manual, Cold Spring Harbor Labs Press.

A “sample” is a subset of its tissues, cells or component parts (e.g., bodily fluids). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule. A “test sample” or “patient sample” means a sample of any type which contains cells or products that have been secreted from cells to be evaluated by the method of the invention, including but not limited to, a sample of isolated cells, a tissue sample or a bodily fluid sample. A “tissue sample”, although similar to a sample of isolated cells, is a section of an organ or tissue of the body which typically includes several cell types, optionally with cytoskeletal structures that hold the cells together. A “cell sample” is a type of “tissue sample”, although term “tissue sample” may more often used to designate a more complex structure than a cell sample. A tissue sample can be obtained by a biopsy, for example, including by cutting, slicing, or a punch. A “bodily fluid sample”, like a tissue sample, contains the cells to be evaluated, and is a fluid obtained by any method suitable for the particular bodily fluid to be sampled. Bodily fluids suitable for sampling include blood, plasma and serum, among others.

“Selectively binds to” means the specific binding of one compound to another (e.g., a formulation of the invention to a cell), wherein the level of binding, as measured by any standard assay, is statistically significantly higher than the background control for the assay.

A “subject”, as used herein, is any organism to which the modified synthetic leptin mRNA formulation of the invention may be administered, whether for experimental, diagnostic, prophylactic or therapeutic purposes. Typical subjects include mammals such as mice, rats, non-human primates and humans.

A “substitution” is a mutation that exchanges one base for another (i.e., a change in a single “chemical letter” such as switching an A to a G). Such a substitution could (i) change a codon to one that encodes a different amino acid and cause a small change in the protein produced; (ii) change a codon to one that encodes the same amino acid and causes no change in the protein produced (“silent mutations”); or (iii) change an amino-acid-coding codon to a single “stop” codon and cause an incomplete protein.

An individual who is “suffering from” a disease, disorder or condition has been diagnosed with or displays one or more symptoms of a disease, disorder or condition.

An individual who is “susceptible to” a disease, disorder or condition has not been diagnosed with or does not exhibit symptoms of the disease, disorder, or condition but harbors a propensity to develop a disease or its symptoms. In some embodiments, an individual who is susceptible to a disease, disorder or condition (for example, obesity) may be characterized by one or more of the following: (i) a genetic mutation associated with development of the disease, disorder or condition; (ii) a genetic polymorphism associated with development of the disease, disorder or condition; (iii) increased or decreased expression or activity of a protein or nucleic acid associated with the disease, disorder or condition; (iv) habits or lifestyles associated with development of the disease, disorder or condition; (v) a family history of the disease, disorder or condition; and (vi) exposure to or infection with a microbe associated with development of the disease, disorder or condition. In some embodiments, an individual who is susceptible to a disease, disorder or condition will develop the disease, disorder or condition. In some embodiments, an individual who is susceptible to a disease, disorder or condition will not develop the disease, disorder or condition.

“Synthetic” means produced, prepared, or manufactured by the human intervention. Synthesis of polynucleotides or polypeptides or other molecules of the invention may be chemical or enzymatic.

A “targeting moiety” is an agent that homes to or preferentially associates or binds to a particular tissue, cell type, receptor, infecting agent or other area of interest. The addition of a targeting moiety to an mRNA delivery composition will enhance the delivery of the mRNA to a desired cell type or location. The addition to, or expression of, a targeting moiety in a cell enhances the localization of that cell to a desired location within an animal or subject.

A “therapeutically effective amount” or “effective amount” is an amount of an agent to be delivered (e.g., nucleic acid, drug, therapeutic agent, diagnostic agent, prophylactic agent, etc.) that is sufficient, when administered to a subject suffering from or susceptible to a disease, disorder or condition, to treat, improve symptoms of, diagnose, prevent, or delay the onset of the disease, disorder or condition, or that will elicit the biological or medical response of a tissue, system, animal or human that is being sought by the researcher, veterinarian, medical doctor or other clinician.

“Treating” is the partially or completely alleviating, ameliorating, improving, relieving, delaying onset of, inhibiting progression of, reducing severity of or reducing incidence of one or more symptoms or features of a particular disease, disorder or condition. For example, “treating” may refer to means reversing or alleviating leptin deficiency or lipodystrophy. “Treatment” means the act of treating, e.g., leptin deficiency or lipodystrophy. Treatment may be administered to a subject who does not exhibit signs of a disease or to a subject who exhibits only early signs of a disease for the purpose of decreasing the risk of developing pathology associated with the disease.

### Leptin Deficiency

The modified synthetic leptin mRNA of the invention is useful for administration to subjects with a leptin deficiency. In principle, any state that leads to leptin deficiency (low circulating leptin level) can be treated by administration of the modified synthetic leptin mRNA of the invention. Subjects with a leptin deficiency can include those with the following conditions: (i) congenital leptin deficiency due to mutation in the leptin gene, (ii) congenital and acquired generalized lipodystrophy; (iii) acquired HIV lipodystrophy, in which the patients have low leptin levels; (iv) hypothalamic amenorrhea, whether the exercise-induced form or the nonathletic form (which is diet induced); (v) weight maintenance following 10% body weight loss (i.e., a metabolic adaptation). Subjects with any of these conditions are in a state of low circulating leptin level and will benefit from administration of the modified synthetic leptin mRNA of the invention for normalizing metabolic disease (diabetes, insulin resistance, hypertriglyceridemia and hyperphagia), menstrual cycle and weight maintenance. See, Kelesidis T et al. (2010) Annals of Internal Medicine 152(2):93-101 and Dardeno T A et al. (2010) Frontiers in Neuroendocrinology 31:377-393.

Congenital leptin deficiency is caused by mutations in the leptin gene. Subjects with congenital leptin deficiency eat voraciously, become morbidly obese and suffer the consequences of obesity including hypogonadism and diabetes.

Lipodystrophy syndrome can be either inherited or acquired, with generalized or partial loss of adipose tissue and a resulting inadequate leptin level in circulation. Patients with lipodystrophy syndrome have degeneration or redistribution of the body's adipose tissue (low adiposity) or both. Patients with lipodystrophy syndrome display severe metabolic phenotype often associated with morbid obesity, such as insulin resistance, diabetes, hypertriglyceridemia and hyperphagia. Patients with severe lipodystrophy are also predisposed to develop cardiomyopathy, acute pancreatitis, cirrhosis, blindness and end stage diabetic renal disease requiring kidney transplantation.

Acquired HIV lipodystrophy affects 15-35% of HIV-infected subjects. Patients with acquired HIV lipodystrophy display metabolic syndrome including insulin resistance, hyperlipidemia and central obesity.

Hypothalamic amenorrhea is a cessation of menstrual cycles that results from dysregulation of a subject's hypothalamic-pituitary-gonal axis. Hypothalamic amenorrhea can be induced by stress, exercise or weight loss.

Obese patients are generally leptin resistant. Following moderate body weight loss leptin sensitivity is partially restored. Thus the administration of the modified synthetic leptin mRNA of the invention allows patients to maintain body weight.

Modified Synthetic Leptin mRNA

The modified synthetic leptin mRNA of the invention encodes a leptin polypeptide, comprises at least one modified nucleoside and has at least the following characteristics: (i) it is generated in vitro and is not isolated from a cell; (ii) it is translatable in a cell (e.g., human cell) in vivo or ex vivo; and (iii) it provokes a significantly reduced innate immune response or interferon response in a subject to whom it is introduced or contacted relative to a non-modified RNA of the same sequence.

The modified synthetic leptin mRNA of the invention can encode a leptin polypeptide that is functionally equivalent to native human leptin protein (SEQ ID NO: 3), as measured by in vivo function (such as by regulating appetite, energy metabolism or neuroendocrine function) or in vitro function (such as by binding functionally to a human leptin receptor (LEPR, CD295)) or both. The modified synthetic leptin mRNA of the invention can encode a leptin polypeptide that is structurally similar to native human leptin protein, as determined by homology to native human protein. The modified synthetic leptin mRNA of the invention can encode a leptin polypeptide that is both functionally equivalent to and structurally similar to native human leptin protein. In specific examples, the modified synthetic leptin mRNA of the invention contains a coding sequence selected from among SEQ ID NOS: 17-20. See EXAMPLE 31. The nucleotide sequences of these four human leptin open reading frames are between 78% and 91% identical to each other. Accordingly, a modified synthetic leptin mRNA of the invention can contain a functional coding region that is at least 78% identical to a coding sequence selected from among SEQ ID NOS: 17-20.

Coding regions for a modified synthetic leptin mRNA of the invention can be constructed by codon optimization of any native leptin coding sequence, including a native human leptin coding sequence. Codon optimization can be performed by commercially available services such as GeneArt® (available from Life Technologies Corporation, Grand Island, N.Y. USA), GENEWHIZ (available from GENEWHIZ, Inc., Cambridge Mass. USA) and GenScript (available from GenScript, Inc., Piscataway N.J. USA).

TABLE 1 provides some polynucleotide and polypeptide sequences useful for the practice of the invention.

TABLE 1
Polynucleotide and Polypeptide Sequences

| SEQ ID NO: | |
|---|---|
| and features | Sequence |
| SEQ ID NO: 1 | (gcc)gccRccAUGG |
| Consensus Kozak | |
| sequence | |
| R is a purine | |
| (adenine or | |
| guanine) three | |
| bases upstream | |
| of the start | |
| codon (AUG), | |
| which is | |
| followed by | |
| another ‘G’. | |
| SEQ ID NO: 2 | ATGCATTGGGGAACCCTGTGCGGATTCT |
| Native DNA sequence | TGTGGCTTTGGCCCTATCTTTTCTATGT |
| corresponding to | CCAAGCTGTGCCCATCCAAAAAGTCCAA |
| Protein Accession | GATGACACCAAAACCCTCATCAAGACAA |
| # NP_000221 | TTGTCACCAGGATCAATGACATTTCACA |
| | CACGCAGTCAGTCTCCTCCAAACAGAAA |
| | GTCACCGGTTTGGACTTCATTCCTGGGC |
| | TCCACCCCATCCTGACCTTATCCAAGAT |
| | GGACCAGACACTGGCAGTCTACCAACAG |
| | ATCCTCACCAGTATGCCTTCCAGAAACG |
| | TGATCCAAATATCCAACGACCTGGAGAA |
| | CCTCCGGGATCTTCTTCACGTGCTGGCC |
| | TTCTCTAAGAGCTGCCACTTGCCCTGGG |
| | CCAGTGGCCTGGAGACCTTGGACAGCCT |
| | GGGGGGTGTCCTGGAAGCTTCAGGCTAC |
| | TCCACAGAGGTGGTGGCCCTGAGCAGGC |
| | TGCAGGGGTCTCTGCAGGACATGCTGTG |
| | GCAGCTGGACCTCAGCCCTGGGTGC |
| SEQ ID NO: 3 | MHWGTLCGFLWLWPYLFYVQAVPIQKVQ |
| Translated human | DDTKTLIKTIVTRINDISHTQSVSSKQK |
| leptin from coding | VTGLDFIPGLHPILTLSKMDQTLAVYQQ |
| sequence (CDS) of | ILTSMPSRNVIQISNDLENLRDLLHVLA |
| the DNA construct | FSKSCHLPWASGLETLDSLGGVLEASGY |
| of SEQ ID NO: 2 | STEVVALSRLQGSLQDMLWQLDLSPGC |
| SEQ ID NO: 4 | GGGAGACGCGUGUUAAAUAACAAAUCUC |
| TEV-hLeptin-01opt-2 | AACACAACAUAUACAAAACAAACGAAUC |
| xhBG-120A | UCAAGCAAUCAAGCAUUCUACUUCUAUU |
| Sequence features: | GCAGCAAUUUAAAUCAUUUCUUUUAAAG |
| Tobacco Etch | CAAAAGCAAUUUUCUGAAAAUUUUCACC |
| Virus (TEV) | AUUUACGAACGAUAGCCGCCACCAUGCA |
| 5′ UTR: 14-154 | CUGGGGAACCCUGUGCGGAUUCCUGUGG |
| Optimal Kozak | CUGUGGCCCUACCUGUUCUAUGUGCAAG |
| sequence: 155-163 | CCGUGCCCAUCCAGAAGGUGCAGGACGA |
| Human leptin | CACCAAGACCCUGAUCAAGACCAUCGUG |
| encoding amino | ACCCGGAUCAACGACAUCAGCCACACCC |
| acids 1-167 of | AGAGCGUGUCCAGCAAGCAGAAAGUGAC |
| Protein Accession | CGGCCUGGACUUCAUCCCCGGCCUGCAC |
| # NP_000221, | CCUAUCCUGACCCUGUCCAAGAUGGACC |
| sequence codon | AGACCCUGGCCGUGUACCAGCAGAUCCU |
| optimized: 164-664 | GACCAGCAUGCCCAGCCGGAACGUGAUC |
| 2 stop | CAGAUCAGCAACGACCUGGAAAACCUGC |
| codons: 665-670 | GGGACCUGCUGCACGUGCUGGCCUUCAG |
| 2 copies of | CAAGAGCUGCCAUCUGCCUUGGGCCAGC |
| human beta-globin | GGCCUGGAAACCCUGGAUUCUCUGGGCG |
| 3′UTR: 689-954 | GAGUGCUGGAAGCCAGCGGCUACUCUAC |
| 120 nucleotide | AGAGGUGGUGGCCCUGAGCAGACUGCAG |
| polyA tail: | GGCAGCCUGCAGGAUAUGCUGUGGCAGC |
| 961-1080 | UGGAUCUGAGCCCCGGCUGCUAAUAGCG |
| | GACCGGCGAUAGAUGAAGCUCGCUUUCU |
| | UGCUGUCCAAUUUCUAUUAAAGGUUCCU |
| | UUGUUCCCUAAGUCCAACUACUAAACUG |
| | GGGGAUAUUAUGAAGGGCCUUGAGCAUC |
| | UGGAUUCUGCCUAAUAAAAAACAUUUAU |
| | UUUCAUUGCAGCUCGCUUUCUUGCUGUC |
| | CAAUUUCUAUUAAAGGUUCCUUUGUUCC |
| | CUAAGUCCAACUACUAAACUGGGGGAUA |
| | UUAUGAAGGGCCUUGAGCAUCUGGAUUC |
| | UGCCUAAUAAAAAACAUUUAUUUUCAUU |
| | GCGGCCGCAAAAAAAAAAAAAAAAAAAA |
| | AAAAAAAAAAAAAAAAAAAAAAAAAAAA |
| | AAAAAAAAAAAAAAAAAAAAAAAAAAAA |
| | AAAAAAAAAAAAAAAAAAAAAAAAAAAA |
| | AAAAAAAAAAAAAAAA |
| SEQ ID NO: 6 | GGGAGACGCGUGUUAAAUAACAAAUCUC |
| TEV-hLeptin-02opt- | AACACAACAUAUACAAAACAAACGAAUC |
| 2xhBG-120A | UCAAGCAAUCAAGCAUUCUACUUCUAUU |
| Sequence features: | GCAGCAAUUUAAAUCAUUUCUUUUAAAG |
| Tobacco Etch Virus | CAAAAGCAAUUUUCUGAAAAUUUUCACC |
| (TEV)5′ UTR: 14-154 | AUUUACGAACGAUAGCCGCCACCAUGCA |
| Optimal Kozak | CUGGGGCACCCUGUGUGGCUUUCUGUGG |
| sequence: 155-163 | CUGUGGCCCUACCUGUUCUACGUGCAGG |
| Human leptin encoding | CCGUGCCCAUCCAGAAGGUGCAGGACGA |
| amino acids 1-167 of | CACCAAGACCCUGAUCAAGACCAUCGUC |
| Protein Accession | ACCAGGAUCAACGACAUCUCCCACACCC |
| # NP_000221, | AGUCCGUGUCCAGCAAGCAAAAGGUGAC |
| sequence codon | CGGACUGGACUUCAUCCCCGGCCUGCAU |
| optimized: 164-664 | CCCAUCCUGACCCUGAGCAAGAUGGACC |
| 2 stop codons: | AGACACUGGCCGUGUACCAACAGAUCCU |
| 665-670 | GACCAGCAUGCCCAGCAGGAACGUGAUC |
| 2 copies of human | CAGAUCUCCAACGACCUGGAGAACCUCA |
| beta-globin | GGGACCUGCUGCACGUGCUGGCCUUCAG |
| 3′UTR: 689-954 | CAAGAGCUGCCAUCUGCCCUGGGCUAGC |
| 120 nucleotide | GGACUGGAGACCCUGGACAGCCUGGGAG |
| polyA tail: | GAGUGCUGGAAGCCAGCGGCUACAGCAC |
| 961-1080 | AGAGGUGGUCGCCCUGAGCAGGCUCCAG |
| | GGCAGCCUGCAGGACAUGCUGUGGCAGC |
| | UGGACCUGAGCCCCGGAUGCUGAUGACG |
| | GACCGGCGAUAGAUGAAGCUCGCUUUCU |
| | UGCUGUCCAAUUUCUAUUAAAGGUUCCU |
| | UUGUUCCCUAAGUCCAACUACUAAACUG |
| | GGGGAUAUUAUGAAGGGCCUUGAGCAUC |
| | UGGAUUCUGCCUAAUAAAAAACAUUUAU |
| | UUUCAUUGCAGCUCGCUUUCUUGCUGUC |
| | CAAUUUCUAUUAAAGGUUCCUUUGUUCC |
| | CUAAGUCCAACUACUAAACUGGGGGAUA |
| | UUAUGAAGGGCCUUGAGCAUCUGGAUUC |
| | UGCCUAAUAAAAAACAUUUAUUUUCAUU |
| | GCGGCCGCAAAAAAAAAAAAAAAAAAAA |
| | AAAAAAAAAAAAAAAAAAAAAAAAAAAA |
| | AAAAAAAAAAAAAAAAAAAAAAAAAAAA |
| | AAAAAAAAAAAAAAAAAAAAAAAAAAAA |
| | AAAAAAAAAAAAAAAA |
| SEQ ID NO: 8 | GGGAGACGCGUGUUAAAUAACAAAUCUC |
| TEV-hLeptin-03opt- | AACACAACAUAUACAAAACAAACGAAUC |
| 2xhBG-120A | UCAAGCAAUCAAGCAUUCUACUUCUAUU |
| Sequence features: | GCAGCAAUUUAAAUCAUUUCUUUUAAAG |
| Tobacco Etch Virus | CAAAAGCAAUUUUCUGAAAAUUUUCACC |
| (TEV) 5′ UTR: 14-154 | AUUUACGAACGAUAGCCGCCACCAUGCA |
| Optimal Kozak | UUGGGGGACACUGUGCGGGUUCCUGUGG |
| sequence: 155-163 | CUGUGGCCCUAUCUGUUCUACGUGCAGG |
| Human leptin encoding | CUGUGCCCAUCCAGAAGGUCCAGGACGA |
| amino acids 1-167 of | CACCAAGACACUGAUCAAAACUAUUGUG |
| Protein Accession | ACCAGGAUCAACGAUAUUAGUCACACCC |
| # NP_000221, | AGUCAGUGAGCUCCAAGCAGAAAGUCAC |
| sequence codon | AGGACUGGACUUCAUCCCCGGCCUGCAU |
| optimized: 164-664 | CCUAUUCUGACACUGUCUAAGAUGGAUC |
| 2 stop codons: | AGACUCUGGCCGUGUACCAGCAGAUCCU |
| 665-670 | GACAUCAAUGCCAAGCCGGAAUGUCAUC |
| 2 copies of human | CAGAUUUCCAACGACCUGGAGAAUCUGA |
| beta-globin | GAGAUCUGCUGCACGUGCUGGCAUUUUC |
| 3′UTR: 689-954 | UAAAAGUUGCCAUCUGCCAUGGGCAAGC |
| 120 nucleotide | GGACUGGAAACUCUGGACUCCCUGGGAG |
| polyA tail: | GGGUCCUGGAGGCAUCCGGAUAUUCUAC |
| 961-1080 | CGAAGUGGUCGCUCUGAGCCGGCUGCAG |
| | GGCUCACUGCAGGAUAUGCUGUGGCAGC |
| | UGGAUCUGUCUCCCGGAUGCUAAUAACG |
| | GACCGGCGAUAGAUGAAGCUCGCUUUCU |
| | UGCUGUCCAAUUUCUAUUAAAGGUUCCU |
| | UUGUUCCCUAAGUCCAACUACUAAACUG |
| | GGGGAUAUUAUGAAGGGCCUUGAGCAUC |
| | UGGAUUCUGCCUAAUAAAAAACAUUUAU |
| | UUUCAUUGCAGCUCGCUUUCUUGCUGUC |
| | CAAUUUCUAUUAAAGGUUCCUUUGUUCC |
| | CUAAGUCCAACUACUAAACUGGGGGAUA |
| | UUAUGAAGGGCCUUGAGCAUCUGGAUUC |
| | UGCCUAAUAAAAAACAUUUAUUUUCAUU |
| | GCGGCCGCAAAAAAAAAAAAAAAAAAAA |
| | AAAAAAAAAAAAAAAAAAAAAAAAAAAA |
| | AAAAAAAAAAAAAAAAAAAAAAAAAAAA |
| | AAAAAAAAAAAAAAAAAAAAAAAAAAAA |
| | AAAAAAAAAAAAAAAA |
| SEQ ID NO: 10 | GGGAGACGCGUGUUAAAUAACAAAUCUC |
| TEV-hLpetin-nonopt- | AACACAACAUAUACAAAACAAACGAAUC |
| 2xhBG-120A | UCAAGCAAUCAAGCAUUCUACUUCUAUU |
| Sequence features: | GCAGCAAUUUAAAUCAUUUCUUUUAAAG |
| Tobacco Etch Virus | CAAAAGCAAUUUUCUGAAAAUUUUCACC |
| (TEV) 5′ UTR: 14-154 | AUUUACGAACGAUAGCCGCCACCAUGCA |
| Optimal Kozak | UUGGGGAACCCUGUGCGGAUUCUUGUGG |
| sequence: 155-163 | CUUUGGCCCUAUCUUUUCUAUGUCCAAG |
| Human leptin encoding | CUGUGCCCAUCCAAAAAGUCCAAGAUGA |
| amino acids 1-167 of | CACCAAAACCCUCAUCAAGACAAUUGUC |
| Protein Accession | ACCAGGAUCAAUGACAUUUCACACACGC |
| # NP_000221: 164-664 | AGUCAGUCUCCUCCAAACAGAAAGUCAC |
| 2 stop codons: | CGGUUUGGACUUCAUUCCUGGGCUCCAC |
| 665-670 | CCCAUCCUGACCUUAUCCAAGAUGGACC |
| 2 copies of human | AGACACUGGCAGUCUACCAACAGAUCCU |
| beta-globin | CACCAGUAUGCCUUCCAGAAACGUGAUC |
| 3′UTR: 689-954 | CAAAUAUCCAACGACCUGGAGAACCUCC |
| 120 nucleotide | GGGAUCUUCUUCACGUGCUGGCCUUCUC |
| polyA tail: | UAAGAGCUGCCACUUGCCCUGGGCCAGU |
| 961-1080 | GGCCUGGAGACCUUGGACAGCCUGGGGG |
| | GUGUCCUGGAAGCUUCAGGCUACUCCAC |
| | AGAGGUGGUGGCCCUGAGCAGGCUGCAG |
| | GGGUCUCUGCAGGACAUGCUGUGGCAGC |
| | UGGACCUCAGCCCUGGGUGCUGAUAACG |
| | GACCGGCGAUAGAUGAAGCUCGCUUUCU |
| | UGCUGUCCAAUUUCUAUUAAAGGUUCCU |
| | UUGUUCCCUAAGUCCAACUACUAAACUG |
| | GGGGAUAUUAUGAAGGGCCUUGAGCAUC |
| | UGGAUUCUGCCUAAUAAAAAACAUUUAU |
| | UUUCAUUGCAGCUCGCUUUCUUGCUGUC |
| | CAAUUUCUAUUAAAGGUUCCUUUGUUCC |
| | CUAAGUCCAACUACUAAACUGGGGGAUA |
| | UUAUGAAGGGCCUUGAGCAUCUGGAUUC |
| | UGCCUAAUAAAAAACAUUUAUUUUCAUU |
| | GCGGCCGCAAAAAAAAAAAAAAAAAAAA |
| | AAAAAAAAAAAAAAAAAAAAAAAAAAAA |
| | AAAAAAAAAAAAAAAAAAAAAAAAAAAA |
| | AAAAAAAAAAAAAAAAAAAAAAAAAAAA |
| | AAAAAAAAAAAAAAAA |
| SEQ ID NO: 12 | GGGAGACGCGUGUUAAAUAACAAAUCUC |
| TEV-mEpo(NcoI)- | AACACAACAUAUACAAAACAAACGAAUC |
| 2xhBG-120A | UCAAGCAAUCAAGCAUUCUACUUCUAUU |
| Sequence features: | GCAGCAAUUUAAAUCAUUUCUUUUAAAG |
| Tobacco Etch Virus | CAAAAGCAAUUUUCUGAAAAUUUUCACC |
| (TEV) 5′ UTR: 14-154 | AUUUACGAACGAUAGCCGCCACCAUGGG |
| Optimal Kozak | CGUGCCCGAAAGACCUACACUCCUGCUG |
| sequence: 155-163 | CUGCUGUCACUGCUGCUGAUCCCUCUGG |
| Mouse erythropoietin | GCCUGCCUGUGCUGUGUGCCCCCCCUAG |
| encoding amino acids | ACUGAUCUGCGACAGCAGAGUGCUGGAA |
| 1-191 of Protein | CGGUACAUCCUGGAAGCCAAAGAGGCCG |
| Accession # | AGAACGUGACGAUGGGAUGUGCCGAGGG |
| NP_031968, sequence | CCCCAGACUGAGCGAGAACAUCACCGUG |
| codon: 164-739 | CCCGACACCAAAGUGAACUUCUACGCCU |
| Stop codons: | GGAAGCGGAUGGAAGUGGAAGAACAGGC |
| 740-742 | CAUCGAAGUGUGGCAGGGCCUGAGCCUG |
| 2 copies of human | CUGAGCGAGGCUAUUCUGCAGGCACAGG |
| beta-globin 3′UTR: | CUCUGCUGGCCAACAGCAGCCAGCCUCC |
| 743-1008 | UGAGACACUGCAGCUGCACAUCGACAAG |
| 120 nucleotide | GCCAUCAGCGGCCUGAGAAGCCUGACCU |
| polyA tail: | CCCUGCUGAGGGUGCUGGGAGCCCAGAA |
| 1009-1128 | AGAACUGAUGAGCCCCCCUGACACCACC |
| | CCCCCUGCUCCUCUGAGAACUCUGACCG |
| | UGGACACCUUCUGCAAGCUGUUCCGGGU |
| | GUACGCCAACUUCCUGCGGGGCAAGCUG |
| | AAGCUGUACACCGGCGAAGUGUGCAGAC |
| | GGGGCGACAGAUGAAGCUCGCUUUCUUG |
| | CUGUCCAAUUUCUAUUAAAGGUUCCUUU |
| | GUUCCCUAAGUCCAACUACUAAACUGGG |
| | GGAUAUUAUGAAGGGCCUUGAGCAUCUG |
| | GAUUCUGCCUAAUAAAAAACAUUUAUUU |
| | UCAUUGCAGCUCGCUUUCUUGCUGUCCA |
| | AUUUCUAUUAAAGGUUCCUUUGUUCCCU |
| | AAGUCCAACUACUAAACUGGGGGAUAUU |
| | AUGAAGGGCCUUGAGCAUCUGGAUUCUG |
| | CCUAAUAAAAAACAUUUAUUUUCAUUGC |
| | AAAAAAAAAAAAAAAAAAAAAAAAAAAA |
| | AAAAAAAAAAAAAAAAAAAAAAAAAAAA |
| | AAAAAAAAAAAAAAAAAAAAAAAAAAAA |
| | AAAAAAAAAAAAAAAAAAAAAAAAAAAA |
| | AAAAAAAA |
| SEQ ID NO: 14 | GATCCGGAGGCCGGAGAATTGTAATACG |
| Entire plasmid | ACTCACTATAGGGAGACGCGTGTTAAAT |
| DNA construct | AACAAATCTCAACACAACATATACAAAA |
| for “Nucleotide | CAAACGAATCTCAAGCAATCAAGCATTC |
| sequence of codon | TACTTCTATTGCAGCAATTTAAATCATT |
| optimized native | TCTTTTAAAGCAAAAGCAATTTTCTGAA |
| human leptin” | AATTTTCACCATTTACGAACGATAGCCG |
| Sequence features: | CCACCATGCACTGGGGAACCCTGTGCGG |
| Human leptin | ATTCCTGTGGCTGTGGCCCTACCTGTTC |
| encoding amino | TATGTGCAAGCCGTGCCCATCCAGAAGG |
| acids 1-167 of | TGCAGGACGACACCAAGACCCTGATCAA |
| Protein Accession | GACCATCGTGACCCGGATCAACGACATC |
| # NP_000221, | AGCCACACCCAGAGCGTGTCCAGCAAGC |
| sequence codon | AGAAAGTGACCGGCCTGGACTTCATCCC |
| optimized: 202-702 | CGGCCTGCACCCTATCCTGACCCTGTCC |
| | AAGATGGACCAGACCCTGGCCGTGTACC |
| | AGCAGATCCTGACCAGCATGCCCAGCCG |
| | GAACGTGATCCAGATCAGCAACGACCTG |
| | GAAAACCTGCGGGACCTGCTGCACGTGC |
| | TGGCCTTCAGCAAGAGCTGCCATCTGCC |
| | TTGGGCCAGCGGCCTGGAAACCCTGGAT |
| | TCTCTGGGCGGAGTGCTGGAAGCCAGCG |
| | GCTACTCTACAGAGGTGGTGGCCCTGAG |
| | CAGACTGCAGGGCAGCCTGCAGGATATG |
| | CTGTGGCAGCTGGATCTGAGCCCCGGCT |
| | GCTAATAGCGGACCGGCGATAGATGAAG |
| | CTCGCTTTCTTGCTGTCCAATTTCTATT |
| | AAAGGTTCCTTTGTTCCCTAAGTCCAAC |
| | TACTAAACTGGGGGATATTATGAAGGGC |
| | CTTGAGCATCTGGATTCTGCCTAATAAA |
| | AAACATTTATTTTCATTGCAGCTCGCTT |
| | TCTTGCTGTCCAATTTCTATTAAAGGTT |
| | CCTTTGTTCCCTAAGTCCAACTACTAAA |
| | CTGGGGGATATTATGAAGGGCCTTGAGC |
| | ATCTGGATTCTGCCTAATAAAAAACATT |
| | TATTTTCATTGCGGCCGCAAAAAAAAAA |
| | AAAAAAAAAAAAAAAAAAAAAAAAAAAA |
| | AAAAAAAAAAAAAAAAAAAAAAAAAAAA |
| | AAAAAAAAAAAAAAAAAAAAAAAAAAAA |
| | AAAAAAAAAAAAAAAAAAAAAAAAAAGA |
| | AGAGCAAGCTTTCGATAGCGCTGTTCGT |
| | AGAAAAAAAAGAAGTAAATAATTACTAC |
| | TTGCCATATAGACTAAATAGCTGCGCNT |
| | AATACATCTACACTTTCTANNATTGACA |
| | AGTGATACGTTGCAAAAGGAGCAACACC |
| | CCACAGACTCGATGACTGCGCAGTCATA |
| | CAGTGAAATTGCCCTAATGTCTTACCTC |
| | TGAAAGGGCTAAACGAAAGTAGAGCACT |
| | ATTCCGCGTAGCTATTTAGTGCGATCTT |
| | TTAGAAATATCAGCCCAGAGAGCTGGGC |
| | TGATAAATATTTTATCCGACAAGACGAA |
| | TTTTGCTCAAATGAGTTAAAACGATGCT |
| | ACCACTATCTGCTGCTTTTACGAGATCA |
| | GCCCACCATTGCATCATCGGACGACGTT |
| | GCTCAAGATAATCACTGCGGTTATAAGC |
| | GCGACGCACCTCATTTTTGTCTACATGA |
| | GCAAGCGCTGCTTCAATGACATCAGGTG |
| | GAAATCCTTCCTCATTGAGTGCCGTACT |
| | GGCGATAGAACGCAAGCCGTGTGAAACA |
| | AGTACACCTCCTAAGCCAGCACGCTTGA |
| | GTGCTGCATTCACTGTTTGGCTATTCAT |
| | TGGTTGGTTGGGCTTGATACGGCTAGGA |
| | AAGATAAATTCTCGGCCACCACTGAGAG |
| | GCTTCATCATTTCCAGAATAGCAAGAGC |
| | CCCATCAGATAGTGGAACCGTATGGTCC |
| | CGGTTCATCTTCATTCGAGCTGCAGGAA |
| | TTTTCCATTCGCTAGCATTGAAATCGAT |
| | CTCATCCCATCGAGCCTCAGCAGCTTCG |
| | GCAGGGCGGGTGATGGTTAGAAGTTGCC |
| | ACATGAACAGGCATCTTGTGGACATGCT |
| | GATACTTGCCGTACGCATGGTGTGCATT |
| | AGCTGCGGAAGTTGATCCGGCCGGATGC |
| | TTGGCATGTTTTTCTTTTGCGGTTTCTC |
| | GAAAGCTTGAGTATTCTATAGTGTCACC |
| | TAAATAGCTTGGCGTAATCATGGTCATA |
| | GCTGTTTCCTGTGTGAAATTGTTATCCG |
| | CTCACAATTCCACACAACATACGAGCCG |
| | GAAGCATAAAGTGTAAAGCCTGGGGTGC |
| | CTAATGAGTGAGCTAACTCACATTAATT |
| | GCGTTGCGCTCACTGCCCGCTTTCCAGT |
| | CGGGAAACCTGTCGTGCCAGCTGCATTA |
| | ATGAATCGGCCAACGCGCGGGGAGAGGC |
| | GGTTTGCGTATTGGGCGCTATTCCGCTT |
| | CCTCGCTCACTGACTCGCTGCGCTCGGT |
| | CGTTCGGCTGCGGCGAGCGGTATCAGCT |
| | CACTCAAAGGCGGTAATACGGTTATCCA |
| | CAGAATCAGGGGATAACGCAGGAAAGAA |
| | CATGTGAGCAAAAGGCCAGCAAAAGGCC |
| | AGGAACCGTAAAAAGGCCGCGTTGCTGG |
| | CGTTTTTCGATAGGCTCCGCCCCCCTGA |
| | CGAGCATCACAAAAATCGACGCTCAAGT |
| | CAGAGGTGGCGAAACCCGACAGGACTAT |
| | AAAGATACCAGGCGTTTCCCCCTGGAAG |
| | CTCCCTCGTGCGCTCTCCTGTTCCGACC |
| | CTGCCGCTTACCGGATACCTGTCCGCCT |
| | TTCTCCCTTCGGGAAGCGTGGCGCTTTC |
| | TCATAGCTCACGCTGTAGGTATCTCAGT |
| | TCGGTGTAGGTCGTTCGCTCCAAGCTGG |
| | GCTGTGTGCACGAACCCCCCGTTCAGCC |
| | CGACCGCTGCGCCTTATCCGGTAACTAT |
| | CGTCTTGAGTCCAACCCGGTAAGACACG |
| | ACTTATCGCCACTGGCAGCAGCCACTGG |
| | TAACAGGATTAGCAGAGCGAGGTATGTA |
| | GGCGGTGCTACAGAGTTCTTGAAGTGGT |
| | GGCCTAACTACGGCTACACTAGAAGGAC |
| | AGTATTTGGTATCTGCGCTCTGCTGAAG |
| | CCAGTTACCTTCGGAAAAAGAGTTGGTA |
| | GCTCTTGATCCGGCAAACAAACCACCGC |
| | TGGTAGCGGTGGTTTTTTTGTTTGCAAG |
| | CAGCAGATTACGCGCAGAAAAAAAGGAT |
| | CTCAAGAAGATCCTTTGATCTTTTCTAC |
| | GGGGTCTGACGCTCAGTGGAACGAAAAC |
| | TCACGTTAAGGGATTTTGGTCATGAGAT |
| | TATCAAAAAGGATCTTCACCTAGATCCT |
| | TTTAAATTAAAAATGAAGTTTTAAATCA |
| | ATCTAAAGTATATATGAGTAAACTTGGT |
| | CTGACAGTTACCAATGCTTAATCAGTGA |
| | GGCACCTATCTCAGCGATCTGTCTATTT |
| | CGTTCATCCATAGTTGCCTGACTCCCCG |
| | TCGTGTAGATAACTACGATACGGGAGGG |
| | CTTACCATCTGGCCCCAGTGCTGCAATG |
| | ATACCGCGAGACCCACGCTCACCGGCTC |
| | CAGATTTATCAGCAATAAACCAGCCAGC |
| | CGGAAGGGCCGAGCGCAGAAGTGGTCCT |
| | GCAACTTTATCCGCCTCCATCCAGTCTA |
| | TTAATTGTTGCCGGGAAGCTAGAGTAAG |
| | TAGTTCGCCAGTTAATAGTTTGCGCAAC |
| | GTTGTTGGCATTGCTACAGGCATCGTGG |
| | TGTCACGCTCGTCGTTTGGTATGGCTTC |
| | ATTCAGCTCCGGTTCCCAACGATCAAGG |
| | CGAGTTACATGATCCCCCATGTTGTGCA |
| | AAAAAGCGGTTAGCTCCTTCGGTCCTCC |
| | GATCGTTGTCAGAAGTAAGTTGGCCGCA |
| | GTGTTATCACTCATGGTTATGGCAGCAC |
| | TGCATAATTCTCTTACTGTCATGCCATC |
| | CGTAAGATGCTTTTCTGTGACTGGTGAG |
| | TACTCAACCAAGTCATTCTGAGAATAGT |
| | GTATGCGGCGACCGAGTTGCTCTTGCCC |
| | GGCGTCAATACGGGATAATACCGCGCCA |
| | CATAGCAGAACTTTAAAAGTGCTCATCA |
| | TTGGAAAACGTTCTTCGGGGCGAAAACT |
| | CTCAAGGATCTTACCGCTGTTGAGATCC |
| | AGTTCGATGTAACCCACTCGTGCACCCA |
| | ACTGATCTTCAGCATCTTTTACTTTCAC |
| | CAGCGTTTCTGGGTGAGCAAAAACAGGA |
| | AGGCAAAATGCCGCAAAAAAGGGAATAA |
| | GGGCGACACGGAAATGTTGAATACTCAT |
| | ACTCTTCCTTTTTCAATATTATTGAAGC |
| | ATTTATCAGGGTTATTGTCTCATGAGCG |
| | GATACATATTTGAATGTATTTAGAAAAA |
| | TAAACAAATAGGGGTTCCGCGCACATTT |
| | CCCCGAAAAGTGCCACCTGACGTCTAAG |
| | AAACCATTATTATCATGACATTAACCTA |
| | TAAAAATAGGCGTATCACGAGGCCCTTT |
| | CGTCTCGCGCGTTTCGGTGATGACGGTG |
| | AAAACCTCTGACACATGCAGCTCCCGGA |
| | GACGGTCACAGCTTGTCTGTAAGCGGAT |
| | GCCGGGAGCAGACAAGCCCGTCAGGGCG |
| | CGTCAGCGGGTGTTGGCGGGTGTCGGGG |
| | CTGGCTTAACTATGCGGCATCAGAGCAG |
| | ATTGTACTGAGAGTGCACCATAAATTCG |
| | AGCTCGGTACCCGGG |
| SEQ ID NO: 16 | GGGAGACGCGUGUUAAAUAACAAAUCUC |
| Non-translatable | AACACAACAUAUACAAAACAAACGAAUC |
| TEV-hLeptin- | UCAAGCAAUCAAGCAUUCUACUUCUAUU |
| 01opt(NT)-2xhBG- | GCAGCAAUUUAAAUCAUUUCUUUUAAAG |
| 120A (codon | CAAAAGCAAUUUUCUGAAAAUUUUCACC |
| optimized leptin | AUUUACGAACGAUAGCCGCCACCGUGCA |
| coding region | CUGGGGAACCCUGUGCGGAUUCCUGUGG |
| rendered non- | CUGUGGCCCUACCUGUUCUCUGUGCAAG |
| translatable | CCGUGCCCAUCCAGAAGGUGCAGGACGA |
| by mutation of | CACCAAGACCCUGAUCAAGACCAUCGUG |
| ATG codons) | ACCCGGAUCAACGACAUCAGCCACACCC |
| Sequence features: | AGAGCGUGUCCAGCAAGCAGAAAGUGAC |
| Tobacco Etch Virus | CGGCCUGGACUUCAUCCCCGGCCUGCAC |
| (TEV) 5′ UTR: 14-154 | CCUAUCCUGACCCUGUCCAAGGUGGACC |
| Optimal Kozak | AGACCCUGGCCGUGUACCAGCAGAUCCU |
| sequence: 155-163 | GACCAGCCUGCCCAGCCGGAACGUGAUC |
| Human leptin encoding | CAGAUCAGCAACGACCUGGAAAACCUGC |
| amino acids 1-167 of | GGGACCUGCUGCACGUGCUGGCCUUCAG |
| Protein Accession | CAAGAGCUGCCAUCUGCCUUGGGCCAGC |
| # NP_000221, | GGCCUGGAAACCCUGGAUUCUCUGGGCG |
| sequence codon | GAGUGCUGGAAGCCAGCGGCUACUCUAC |
| optimized but with | AGAGGUGGUGGCCCUGAGCAGACUGCAG |
| AUG codons mutated | GGCAGCCUGCAGGAUGUGCUGUGGCAGC |
| to render mRNA | UGGAUCUGAGCCCCGGCUGCUAAUAGCG |
| non-translatable: | GACCGGCGAUAGAUGAAGCUCGCUUUCU |
| 164-664 | UGCUGUCCAAUUUCUAUUAAAGGUUCCU |
| 2 stop codons: | UUGUUCCCUAAGUCCAACUACUAAACUG |
| 665-670 | GGGGAUAUUAUGAAGGGCCUUGAGCAUC |
| 2 copies of human | UGGAUUCUGCCUAAUAAAAAACAUUUAU |
| beta-globin | UUUCAUUGCAGCUCGCUUUCUUGCUGUC |
| 3′UTR: 689-954 | CAAUUUCUAUUAAAGGUUCCUUUGUUCC |
| 120 nucleotide | CUAAGUCCAACUACUAAACUGGGGGAUA |
| polyA tail: | UUAUGAAGGGCCUUGAGCAUCUGGAUUC |
| 961-1080 | UGCCUAAUAAAAAACAUUUAUUUUCAUU |
| | GCGGCCGCAAAAAAAAAAAAAAAAAAAA |
| | AAAAAAAAAAAAAAAAAAAAAAAAAAAA |
| | AAAAAAAAAAAAAAAAAAAAAAAAAAAA |
| | AAAAAAAAAAAAAAAAAAAAAAAAAAAA |
| | AAAAAAAAAAAAAAAA |
| SEQ ID NO: 17 | AUGCACUGGGGAACCCUGUGCGGAUUCC |
| Codon optimized | UGUGGCUGUGGCCCUACCUGUUCUAUGU |
| coding sequence | GCAAGCCGUGCCCAUCCAGAAGGUGCAG |
| of SEQ ID NO: 4 | GACGACACCAAGACCCUGAUCAAGACCA |
| | UCGUGACCCGGAUCAACGACAUCAGCCA |
| | CACCCAGAGCGUGUCCAGCAAGCAGAAA |
| | GUGACCGGCCUGGACUUCAUCCCCGGCC |
| | UGCACCCUAUCCUGACCCUGUCCAAGAU |
| | GGACCAGACCCUGGCCGUGUACCAGCAG |
| | AUCCUGACCAGCAUGCCCAGCCGGAACG |
| | UGAUCCAGAUCAGCAACGACCUGGAAAA |
| | CCUGCGGGACCUGCUGCACGUGCUGGCC |
| | UUCAGCAAGAGCUGCCAUCUGCCUUGGG |
| | CCAGCGGCCUGGAAACCCUGGAUUCUCU |
| | GGGCGGAGUGCUGGAAGCCAGCGGCUAC |
| | UCUACAGAGGUGGUGGCCCUGAGCAGAC |
| | UGCAGGGCAGCCUGCAGGAUAUGCUGUG |
| | GCAGCUGGAUCUGAGCCCCGGCUGC |
| SEQ ID NO: 18 | AUGCACUGGGGCACCCUGUGUGGCUUUC |
| Codon optimized | UGUGGCUGUGGCCCUACCUGUUCUACGU |
| coding sequence | GCAGGCCGUGCCCAUCCAGAAGGUGCAG |
| of SEQ ID NO: 6 | GACGACACCAAGACCCUGAUCAAGACCA |
| | UCGUCACCAGGAUCAACGACAUCUCCCA |
| | CACCCAGUCCGUGUCCAGCAAGCAAAAG |
| | GUGACCGGACUGGACUUCAUCCCCGGCC |
| | UGCAUCCCAUCCUGACCCUGAGCAAGAU |
| | GGACCAGACACUGGCCGUGUACCAACAG |
| | AUCCUGACCAGCAUGCCCAGCAGGAACG |
| | UGAUCCAGAUCUCCAACGACCUGGAGAA |
| | CCUCAGGGACCUGCUGCACGUGCUGGCC |
| | UUCAGCAAGAGCUGCCAUCUGCCCUGGG |
| | CUAGCGGACUGGAGACCCUGGACAGCCU |
| | GGGAGGAGUGCUGGAAGCCAGCGGCUAC |
| | AGCACAGAGGUGGUCGCCCUGAGCAGGC |
| | UCCAGGGCAGCCUGCAGGACAUGCUGUG |
| | GCAGCUGGACCUGAGCCCCGGAUGC |
| SEQ ID NO: 19 | AUGCAUUGGGGGACACUGUGCGGGUUCC |
| Codon optimized | UGUGGCUGUGGCCCUAUCUGUUCUACGU |
| coding sequence | GCAGGCUGUGCCCAUCCAGAAGGUCCAG |
| of SEQ ID NO: 8 | GACGACACCAAGACACUGAUCAAAACUA |
| | UUGUGACCAGGAUCAACGAUAUUAGUCA |
| | CACCCAGUCAGUGAGCUCCAAGCAGAAA |
| | GUCACAGGACUGGACUUCAUCCCCGGCC |
| | UGCAUCCUAUUCUGACACUGUCUAAGAU |
| | GGAUCAGACUCUGGCCGUGUACCAGCAG |
| | AUCCUGACAUCAAUGCCAAGCCGGAAUG |
| | UCAUCCAGAUUUCCAACGACCUGGAGAA |
| | UCUGAGAGAUCUGCUGCACGUGCUGGCA |
| | UUUUCUAAAAGUUGCCAUCUGCCAUGGG |
| | CAAGCGGACUGGAAACUCUGGACUCCCU |
| | GGGAGGGGUCCUGGAGGCAUCCGGAUAU |
| | UCUACCGAAGUGGUCGCUCUGAGCCGGC |
| | UGCAGGGCUCACUGCAGGAUAUGCUGUG |
| | GCAGCUGGAUCUGUCUCCCGGAUGC |
| SEQ ID NO: 20 | AUGCAUUGGGGAACCCUGUGCGGAUUCU |
| Coding sequence | UGUGGCUUUGGCCCUAUCUUUUCUAUGU |
| of SEQ ID NO: 10 | CCAAGCUGUGCCCAUCCAAAAAGUCCAA |
| Corresponding to | GAUGACACCAAAACCCUCAUCAAGACAA |
| human leptin | UUGUCACCAGGAUCAAUGACAUUUCACA |
| encoding amino | CACGCAGUCAGUCUCCUCCAAACAGAAA |
| acids 1-167 of | GUCACCGGUUUGGACUUCAUUCCUGGGC |
| Protein Accession | UCCACCCCAUCCUGACCUUAUCCAAGAU |
| # NP_000221 | GGACCAGACACUGGCAGUCUACCAACAG |
| | AUCCUCACCAGUAUGCCUUCCAGAAACG |
| | UGAUCCAAAUAUCCAACGACCUGGAGAA |
| | CCUCCGGGAUCUUCUUCACGUGCUGGCC |
| | UUCUCUAAGAGCUGCCACUUGCCCUGGG |
| | CCAGUGGCCUGGAGACCUUGGACAGCCU |
| | GGGGGGUGUCCUGGAAGCUUCAGGCUAC |
| | UCCACAGAGGUGGUGGCCCUGAGCAGGC |
| | UGCAGGGGUCUCUGCAGGACAUGCUGUG |
| | GCAGCUGGACCUCAGCCCUGGGUGC |
| SEQ ID NO: 21 | GGGAGACGCGUGUUAAAUAACAAAUCUC |
| Non-translatable | AACACAACAUAUACAAAACAAACGAAUC |
| TEV-hlgGH1-59- | UCAAGCAAUCAAGCAUUCUACUUCUAUU |
| hLeptin-GAopt(NT)- | GCAGCAAUUUAAAUCAUUUCUUUUAAAG |
| 2xhBG-120A (codon | CAAAAGCAAUUUUCUGAAAAUUUUCACC |
| optimized N-terminal | AUUUACGAACGAUAGCCGCCACCCUGGA |
| Fc-Leptin fusion | CUGGAUCUGGCGCAUCCUGUUUCUCGUG |
| coding region | GGAGCCGCCACAGGCGCCCACAGCGAUA |
| rendered non- | AGACCCACACCUGUCCUCCCUGCCCUGC |
| translatable by | CCCUGAAGCUGCUGGCGGACCUAGCGUG |
| mutation of ATG | UUCCUGUUCCCCCCAAAGCCCAAGGACA |
| codons) | CCCUGGUGAUCAGCCGGACCCCCGAAGU |
| Sequence features: | GACCUGCGUGGUGGUGGCUGUGUCCCAC |
| Tobacco Etch Virus | GAGGACCCUGAAGUGAAGUUCAAUUGGU |
| (TEV) 5′ UTR: 14-154 | ACGUGGACGGCGUGGAAGUGCACAACGC |
| Optimal Kozak | CAAGACCAAGCCCAGAGAGGAACAGUAC |
| sequence: 155-163 | AACAGCACCUACCGGGUGGUGUCCGUGC |
| Human IgGH1-58 signal | UGACCGUGCUGCACCAGGACUGGCUGAA |
| peptide, human IgG1 | CGGCAAAGAGUACAAGUGCAAGGUGUCC |
| constant region, and | AACAAGGCCCUGCCAGCCCCCAUCGAGA |
| leptin encoding | AAACCAUCAGCAAGGCCAAGGGCCAGCC |
| amino acids 1-167 of | CCGCGAACCCCAGGUGUACACACUGCCC |
| Protein Accession # | CCUAGCCGGGAAGAGGUGACCAAGAACC |
| NP_000221, sequence | AGGUGUCCCUGACCUGUCUCGUGAAGGG |
| codon optimized but | CUUCUACCCCUCCGAUAUCGCCGUGGAA |
| with AUG codons | UGGGAGAGCAACGGCCAGCCCGAGAACA |
| mutated to render | ACUACAAGACCACCCCCCCUGUGCUGGA |
| mRNA non-translatable: | CAGCGACGGCUCAUUCUUCCUGUACAGC |
| 164-1346 | AAGCUGACAGUGGACAAGAGCCGGUGGC |
| 2 stop codons: | AGCAGGGCAACGUGUUCAGCUGCAGCGU |
| 1346-1351 | GCUGCACGAGGCCCUGCACAACCACUAC |
| 2 copies of human | ACCCAGAAGUCCCUGAGCCUGAGCCCUG |
| beta-globin | GCAAGGGCAGCGUGCCAAUCCAGAAGGU |
| 3′UTR:1370-1635 | GCAGGACGACACCAAGACCCUGAUCAAG |
| 120 nucleotide | ACCAUCGUGACCCGGAUCAACGACAUCA |
| polyA tail: | GCCACACCCAGAGCGUGUCCAGCAAGCA |
| 1642-1761 | GAAAGUGACCGGCCUGGACUUCAUCCCC |
| | GGCCUGCACCCUAUCCUGACCCUGUCCA |
| | AGGUGGACCAGACCCUGGCCGUGUACCA |
| | GCAGAUCCUGACCAGCCUGCCCAGCCGG |
| | AACGUGAUCCAGAUCAGCAACGACCUGG |
| | AAAACCUGCGGGACCUGCUGCACGUGCU |
| | GGCCUUCAGCAAGAGCUGCCAUCUGCCU |
| | UGGGCCAGCGGCCUGGAAACCCUGGAUU |
| | CUCUGGGCGGAGUGCUGGAAGCCAGCGG |
| | CUACUCUACAGAGGUGGUGGCCCUGAGC |
| | AGACUGCAGGGCAGCCUGCAGGAUGUGC |
| | UGUGGCAGCUGGAUCUGAGCCCCGGCUG |
| | UUAAUAGCGGACCGGCGAUAGAUGAAGC |
| | UCGCUUUCUUGCUGUCCAAUUUCUAUUA |
| | AAGGUUCCUUUGUUCCCUAAGUCCAACU |
| | ACUAAACUGGGGGAUAUUAUGAAGGGCC |
| | UUGAGCAUCUGGAUUCUGCCUAAUAAAA |
| | AACAUUUAUUUUCAUUGCAGCUCGCUUU |
| | CUUGCUGUCCAAUUUCUAUUAAAGGUUC |
| | CUUUGUUCCCUAAGUCCAACUACUAAAC |
| | UGGGGGAUAUUAUGAAGGGCCUUGAGCA |
| | UCUGGAUUCUGCCUAAUAAAAAACAUUU |
| | AUUUUCAUUGCGGCCGCAAAAAAAAAAA |
| | AAAAAAAAAAAAAAAAAAAAAAAAAAAA |
| | AAAAAAAAAAAAAAAAAAAAAAAAAAAA |
| | AAAAAAAAAAAAAAAAAAAAAAAAAAAA |
| | AAAAAAAAAAAAAAAAAAAAAAAAA |

The modified synthetic leptin mRNA of the invention can contain multiple elements that initiate and boost translation, antagonize deadenylation and RNA chain degradation, and prevent induction of inflammatory responses.

The modified synthetic leptin mRNA can begin at the 5′ end with an mRNA cap that is enzymatically synthesized after the mRNA has been transcribed by an RNA polymerase in vitro. The mRNA cap facilitates translation initiation while avoiding recognition of the mRNA as foreign and protects the mRNA from 5′ exonuclease mediated degradation. The 5′ cap can comprise a modified guanine nucleotide that is linked to the 5′ end of an RNA molecule using a 5′-5′ triphosphate linkage. The 5′ cap can also be a 5′ cap analog, such as 5′ diguanosine cap, tetraphosphate cap analogs having a methylene-bis(phosphonate) moiety (see e.g., Rydzik A M et al. (2009) Org. Biomol. Chem. 7(22):4763-76), dinucleotide cap analogs having a phosphorothioate modification (see e.g., Kowalska J et al. (2008) RNA 14(6):1119-1131), cap analogs having a sulfur substitution for a non-bridging oxygen (see e.g., Grudzien-Nogalska E et al. (2007) RNA 13(10): 1745-1755), N7-benzylated dinucleoside tetraphosphate analogs (see e.g., Grudzien E et al. (2004) RNA 10(9):1479-1487), or anti-reverse cap analogs (see e.g., Jemielity J et al., (2003) RNA 9(9): 1108-1122 and Stepinski J et al. (2001) RNA 7(10):1486-1495). In one such embodiment, the 5′ cap analog is a 5′ diguanosine cap. See also, the method for capping by New England Biolabs (Beverly Mass. USA) shown in EXAMPLE 2. In some embodiments, the modified synthetic leptin mRNA of the invention does not comprise a 5′ triphosphate.

Next can be a synthetic 5′ untranslated region (UTR) of eukaryotic or viral origin that has minimal secondary structure and boosts cap-dependent translation efficiency either by enhancing ribosome recruitment or improving the efficiency of ribosome scanning from the cap to the Kozak sequence and start codon.

In some embodiments, the modified synthetic leptin mRNA of the invention includes a Kozak sequence. The “Kozak sequence” refers to a sequence on eukaryotic mRNA having the consensus (gcc)gccRccAUGG (SEQ ID NO: 1), where R is a purine (adenine or guanine) three bases upstream of the start codon (AUG), which is followed by another “G”. The Kozak consensus sequence is recognized by the ribosome to initiate translation of a polypeptide. Typically, initiation occurs at the first AUG codon encountered by the translation machinery that is proximal to the 5′ end of the transcript. The presence of a Kozak sequence near the AUG codon strengthens that codon as the initiating site of translation, such that translation of the correct polypeptide occurs. In some such embodiments, the Kozak sequence comprises one or more modified nucleosides.

This is followed by an open reading frame that encodes a polypeptide that is functionally equivalent to native human leptin protein. While exemplary open reading frames are disclosed herein, other open reading frames that produce a functional leptin protein can be used.

The open reading frame can be followed by a synthetic 3′ untranslated region (3′UTR) sequence of eukaryotic or viral origin. The 3′ UTR can be selected from an mRNA known to have high stability in a cell. For example, the synthetic 3′UTR sequence can be murine α-globin 3′ UTR or a murine β-globin 3′ UTR as described in Intl. Pat. Appl. No. WO 2007/036366 to the Johannes Gutenberg-Universitat Mainz. This 3′UTR can be followed by another synthetic 3′UTR sequence coding for a poly-adenosine tail that, together, antagonizes degradation of the mRNA and enhances its translation in cells.

The modified synthetic leptin mRNA of the invention can include modifications to prevent rapid degradation by endo- and exo-nucleases and to avoid or reduce the cell's innate immune or interferon response to the RNA. Modifications can include, for example, (i) end modifications, e.g., 5′ end modifications (phosphorylation dephosphorylation, conjugation, inverted linkages, etc.), 3′ end modifications (conjugation, DNA nucleotides, inverted linkages, etc.), (ii) base modifications, e.g., replacement with modified bases, stabilizing bases, destabilizing bases, or bases that base pair with an expanded repertoire of partners, or conjugated bases, (iii) sugar modifications (e.g., at the 2′ position or 4′ position) or replacement of the sugar, as well as (iv) internucleoside linkage modifications, including modification or replacement of the phosphodiester linkages. Many of these modifications have been used to modify siRNA molecules. Some base modifications are useful for reducing immune response to administered mRNA. See, Kariko K et al. (2005) Immunity 23: 165-175.

The modified synthetic leptin mRNA of the invention can be prepared according to any available technique such as enzymatic synthesis by in vitro transcription, enzymatic or chemical cleavage of a longer precursor, etc. Methods of synthesizing RNAs are known in the art. See, e.g., early references for the in vitro transcription of long RNA from plasmid template using recombinant T7 RNA polymerase, such as Chapman K A and Wells R D (1982) “Bacteriophage T7 late promoters; construction and in vitro transcription properties of deletion mutants.” Nucleic Acids Research 10(20): 6331-6340 and Schenborn E T and Mierendorf R C (1985) “A novel transcription property of SP6 and T7 RNA polymerases: dependence on template structure.” Nucleic Acids Research 13(17): 6223-6236. Transcription methods are described further herein in EXAMPLE 2.

The modified synthetic leptin mRNA of the invention can be generated by in vitro transcription of a leptin DNA template. Methods for generating templates are well known to those of skill in the art using standard molecular cloning techniques. The mRNA is synthesized in vitro with ribonucleotides containing modifications (for example, the substitution of uridine with pseudouridine) that have the effect of enhancing mRNA translation and stability and avoiding the induction of inflammatory responses. The transcribed modified synthetic leptin mRNA can be modified further post-transcription, e.g., by adding a cap or other functional group.

For transcription, the modified nucleotides can be recognized as substrates by at least one RNA polymerase enzyme. Generally, RNA polymerase enzymes can tolerate a range of nucleoside base modifications. Ribose and phosphate-modified nucleosides or nucleoside analogs are known in the art that permit transcription by RNA polymerases. Polymerases that accept modified nucleosides are known to those of skill in the art. See, U.S. Pat. No. 8,278,036 to Kariko et al. For example, the RNA polymerase can be a phage RNA polymerase. The modified nucleotides accepted by RNA polymerases include pseudouridine (ψ), 5-methyl-uridine (m5U), 2-thiouridine (s2U), 6-methyl-adenine (m6A), and 5-methyl-cytidine (m5C) are known to be compatible with transcription using phage RNA polymerases, while N1-methylguanosine, N1-methyladenosine, N7-methylguanosine, 2′-O-methyluridine, and 2′-O-methylcytidine are not.

Modified polymerases can be used to generate the modified synthetic leptin mRNA of the invention. A modified (mutant, R425C) T7 RNA polymerase that efficiently incorporates 2′-O-methyl-modified ribonucleotide 5′-triphosphates has recently been described by Ibach J et al. (2013) “Identification of a T7 RNA polymerase variant that permits the enzymatic synthesis of fully 2′-O-methyl-modified mRNA.” J. Biotechnology 167:287-295.

The transcription or other synthesis of the modified synthetic leptin mRNA of the invention can be monitored by any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

The modified synthetic leptin mRNA of the invention can be translated by the translation machinery of a eukaryotic cell. Nucleoside modifications other than pseudouridine (ψ) can interfere with translation. One of skill in the art can test a modified synthetic leptin mRNA of the invention for its ability to undergo translation and translation efficiency using an in vitro translation assay (e.g., a rabbit reticulocyte lysate assay, a reporter activity assay, or measurement of a radioactive label in the translated protein) and detecting the amount of the polypeptide produced using SDS-PAGE, Western blot, ELISA, or immunochemistry assays, etc. The translation of a modified synthetic leptin mRNA of the invention comprising a candidate modification is compared to the translation of an RNA lacking the candidate modification, such that if the translation of the modified synthetic leptin mRNA of the invention having the candidate modification remains the same or is increased then the candidate modification is contemplated for use with the compositions and methods described herein.

### Formulations

The modified synthetic leptin mRNA is formulated with a delivery agent that has several advantageous features. The delivery agent protects the modified synthetic leptin mRNA from degradation. The delivery agent can also assist in delivering the modified synthetic leptin mRNA to an intended tissue or cell type, which results in increased translation of leptin protein and increased levels of leptin protein in a subject's circulatory system.

mRNA on its own is not efficiently delivered to the cytoplasm of cells and is vulnerable to degradation by ribonucleases. mRNA is also strongly negatively charged and hydrophilic, which makes entry of mRNA into the cytoplasm of cells from the extracellular space or endocytic vesicles very unlikely. Thus, the delivery of mRNA to cells in vivo for therapeutic purposes requires the use of a formulation, where the delivery agent in the formulation both protects the mRNA from ribonucleases and facilitates delivery of mRNA to the cytoplasm of cells.

A modified synthetic leptin mRNA formulation can include a delivery agent such as a nanoparticle, a dendrimer, a polymer, an emulsion, a liposome, a cationic lipid, a non-cationic lipid, an anionic lipid, a charged lipid or a penetration enhancer. Positively charged cationic delivery systems facilitate binding of a modified synthetic leptin mRNA of the invention (negatively charged polynucleotides) and also enhances interactions at the negatively charged cell membrane to permit efficient cellular uptake. Cationic lipids, dendrimers or polymers can either be bound to modified synthetic RNAs or induced to form a vesicle or micelle. See e.g., Kim S H et al. (2008) Journal of Controlled Release 129(2):107-116) that encases the modified RNA. Methods for making and using cationic-modified RNA complexes are well within the abilities of those skilled in the art (see e.g., Sorensen D R et al. (2003) J. Mol. Biol. 327:761-766; Verma U N et al. (2003) Clin. Cancer Res. 9:1291-1300; Arnold A S et al. (2007) J. Hypertens. 25:197-205. Guidance for how to assess the lipidic and polymeric carriers for nucleic acid delivery is provided by Nguyen J and Szoka F C (2012) Accounts of Chemical Research 45(7): 1153-1162.

Delivery agents for transfection or lipofection. In some embodiments, a modified synthetic leptin mRNA of the invention can be introduced to a cell or tissue by transfection or lipofection. Suitable delivery agents for transfection or lipofection include, for example, calcium phosphate, DEAE dextran, lipofectin, lipofectamine, DIMRIE C™, Superfect™, and Effectin™ (Qiagen™), Unifectin™ Maxifectin™ DOTMA, DOGS™ (Transfectam; dioctadecylamidoglycylspermine), DOPE (1,2-dioleoyl-sn-glycero-3-phosphoethanolamine), DOTAP (1,2-dioleoyl-3-trimethylammonium propane), DDAB (dimethyl dioctadecylammonium bromide), DHDEAB (N,N-di-n-hexadecyl-N,N-dihydroxyethyl ammonium bromide), HDEAB (N-n-hexadecyl-N,N-dihydroxyethylammonium bromide), polybrene, poly(ethylenimine) (PEI), and the like. See, e.g., Banerjee et al. (1999) Med. Chem. 42:4292-99; Godbey et al. (1999) Gene Ther. 6:1380-88; Kichler et al. (1998) Gene Ther. 5:855-60; Birchaa et al. (1999) J. Pharm. 183:195-207.

Cell Penetration Enhancers.

In some embodiments, the modified synthetic leptin mRNA of the invention is formulated in conjunction with one or more penetration enhancers, surfactants or chelators. Suitable surfactants include fatty acids and or esters and salts thereof, bile acids and salts thereof. Suitable bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Suitable fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g., sodium). In some embodiments, combinations of penetration enhancers are used, for example, fatty acids/salts in combination with bile acids/salts. One exemplary combination is the sodium salt of lauric acid, capric acid and UDCA. Other penetration enhancers include polyoxyethylene-9-lauryl ether, polyoxyethylene-20-cetyl ether.

Liposomes.

A number of liposomes containing nucleic acids are known in the art. Intl. Pat. Appl. No. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes can include an RNA molecule. U.S. Pat. No. 5,665,710 to Rahman et al. describes certain methods of encapsulating oligodeoxynucleotides in liposomes. Intl. Pat. Appl. No. WO 97/04787 to Love et al. discloses liposomes comprising RNAi molecules. Methods for preparing a liposome formulation comprising a nucleic acid can also be found in e.g., U.S. Pat. Nos. 6,011,020; 6,074,667; 6,110,490; 6,147,204; 6, 271, 206; 6,312,956; 6,465,188; 6,506,564; 6,750,016; and 7,112,337. Each of these approaches can be used to provide delivery of a modified synthetic leptin mRNA of the invention to a cell.

Polymers.

The modified synthetic leptin mRNA of the invention can be formulated using natural or synthetic polymers, as shown in the Intl. Pat. Appl. No. WO 2013/090186A1. Examples of polymers which can be used for delivery include, Dynamic POLYCONJUGATE™ formulations from MIRUS® Bio (Madison Wis. USA) and Roche Madison (Madison Wis. USA), PHASERX™ polymer formulations such as SMARTT POLYMER TECHNOLOGY™ (PhaseRx, Seattle Wash. USA), DMRI/DOPE, poloxamer, VAXFECTIN® adjuvant from Vical (San Diego Calif. USA), chitosan, cyclodextrin from Calando Pharmaceuticals (Pasadena Calif. USA), dendrimers and poly(lactic-co-glycolic acid) (PLGA) polymers, RONDEL™ (RNAi/Oligonucleotide Nanoparticle Delivery) polymers (Arrowhead Research Corporation, Pasadena Calif. WA) and pH responsive co-block polymers such as PHASERX™ (Seattle Wash. USA). See, U.S. Pat. Nos. 6,835,393, 7,374,778, 7,737,108, 7,718,193 and 8,003,129. See also European patent EP1044021B1.

An example of poly(lactic-co-glycolide) (PLGA) formulations includes PLGA injectable depots, e.g., ELIGARD®.

Many of these polymer approaches have demonstrated efficacy in delivering oligonucleotides in vivo into a cell cytoplasm. These approaches have been reviewed by deFougerolles (2008) Hum Gene Ther. 19: 125-132. Two polymer approaches that have yielded in vivo delivery of nucleic acids, in particular small interfering RNA (siRNA), are dynamic polyconjugates and cyclodextrin-based nanoparticles. The first of these delivery approaches uses dynamic polyconjugates and has been shown in vivo in mice to effectively deliver siRNA. Rozema et al. (2007) Proc. Natl. Acad. Sci. USA 104: 12982-12887. This approach is a multicomponent polymer system whose features include a membrane-active polymer to which nucleic acid can be covalently coupled by a disulfide bond and where both PEG (for charge masking) and N-acetylgalactosamine (a targeting moiety for hepatocyte targeting) groups are linked by pH-sensitive bonds. Rozema et al. (2007) Proc. Natl. Acad. Sci. USA 104: 12982-12887.

Another polymer approach involves using transferrin-targeted cyclodextrin-containing polycation nanoparticles. These nanoparticles have demonstrated targeted silencing of the EWS-FLI1 gene product in transferrin receptor-expressing Ewing's sarcoma tumor cells. See, Hu-Lieskovan et al. (2005) Cancer Res. 65: 8984-8982. siRNA formulated in these nanoparticles was well tolerated in non-human primates. See, Heidel et al. (2007) Proc. Natl. Acad. Sci. USA 104:5715-21. Polymer formulations can be selectively targeted through expression of different ligands, such as folate, transferrin, and N-acetylgalactosamine (GaINAc). See, Benoit et al. (2011) Biomacromolecules 12:2708-2714; Rozema et al. (2007) Proc. Natl. Acad. Sci. USA 104: 12982-12887; Davis (2009) Mol. Pharm. 6: 659-668; Davis (2010) Nature 464: 1067-1070.

The delivery agent can include at least one polymer such as polyethenes, polyethylene glycol (PEG), poly(l-lysine)(PLL), PEG grafted to PLL, cationic lipopolymer, biodegradable cationic lipopolymer, polyethyleneimine (PEI), cross-linked branched poly(alkylene imines), a polyamine derivative, a modified poloxamer, a biodegradable polymer, biodegradable block copolymer, biodegradable random copolymer, biodegradable polyester copolymer, biodegradable polyester block copolymer, biodegradable polyester block random copolymer, linear biodegradable copolymer, poly[a-(4-aminobutyl)-L-glycolic acid) (PAGA), biodegradable cross-linked cationic multi-block copolymers, polycarbonates, polyanhydrides, polyhydroxyacids, polypropylfumerates, polycaprolactones, polyamides, polyacetals, polyethers, polyesters, poly(ortho esters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester), acrylic polymers, amine-containing polymers or combinations thereof.

The modified synthetic leptin mRNA of the invention can be formulated with polymers by one of skill in the formulation art, using guidance provided in the scientific literature, patents and published patent applications. For example, the modified synthetic leptin mRNA of the invention can be formulated with the polymeric compound of PEG grafted with PLL as described in U.S. Pat. No. 6,177,274 and used for in vivo delivery. For another example, the modified synthetic leptin mRNA of the invention can be suspended in a solution or medium with a cationic polymer, in a dry pharmaceutical composition or in a solution that is capable of being dried as described in U.S. Pat. Appl. Nos. 2009/0042829 and 2009/0042825.

The modified synthetic leptin mRNA of the invention can also be formulated with a PLGA-PEG block copolymer as described by U.S. Pat. Appl. No. 2012/0004293 and U.S. Pat. No. 8,236,330. The modified synthetic leptin mRNA of the invention can be formulated with a diblock copolymer of PEG and PLA or PEG and PLGA as described in U.S. Pat. No. 8,246,968. The polymers described herein can be conjugated to a lipid-terminating PEG. For example, PLGA can be conjugated to a lipid-terminating PEG forming PLGA-DSPE-PEG. PEG conjugates are described in Intl. Pat. Appl. No. WO 2008/103276.

The modified synthetic leptin mRNA of the invention can be formulated with a polyamine derivative delivery agent to be included in an implantable or injectable device, as described by U.S. Pat. Appl. No. 2010/0260817. The modified synthetic leptin mRNA of the invention can be formulated with a polyamine derivative described in U.S. Pat. Appl. No. 2010/0260817.

The modified synthetic leptin mRNA of the invention can be formulated with acrylic acid, methacrylic acid, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), polycyanoacrylates and combinations thereof. Formulations of the modified synthetic leptin mRNA of the invention can include at least one amine—containing polymer such as polylysine, polyethylene imine, poly(amidoamine) dendrimers or combinations thereof.

The modified synthetic leptin mRNA of the invention can be formulated with at least one polymer described in Intl. Pat. Appl. Nos. WO 2011/15862, WO 2012/082574 and WO 2012/068187. Moreover, the modified synthetic leptin mRNA of the invention can be formulated in a delivery agent including a poly(alkylene imine), a biodegradable cationic lipopolymer, a biodegradable block copolymer, a biodegradable polymer, or a biodegradable random copolymer, a biodegradable polyester block copolymer, a biodegradable polyester polymer, a biodegradable polyester random copolymer, a linear biodegradable copolymer, PAGA, a biodegradable cross-linked cationic multi-block copolymer or combinations thereof. The biodegradable cationic lipopolymer can be made by methods described in U.S. Pat. No. 6,696,038 or U.S. Pat. Appl. Nos. 2003/0073619 and 2004/0142474. The poly(alkylene imine) can be made using methods described in U.S. Pat. Appl. No. 2010/0004315. The biodegradable polymer, biodegradable block copolymer, the biodegradable random copolymer, biodegradable polyester block copolymer, biodegradable polyester polymer, or biodegradable polyester random copolymer can be made using methods described in U.S. Pat. Nos. 6,517,869 or 6,267,987. The linear biodegradable copolymer can be made using methods known in the art, e.g., as described in U.S. Pat. No. 6,652,886. The PAGA polymer can be made using methods described in U.S. Pat. No. 6,217,912. The PAGA polymer can be copolymerized to form a copolymer or block copolymer with polymers such as but not limited to, poly-L-lysine, polyargine, polyornithine, histones, avidin, protamines, polylactides and poly(lactide-co-glycolides). The biodegradable cross-linked cationic multi-block copolymers can be made by methods described in U.S. Pat. No. 8,057,821 or U.S. Pat. Appl. No. 2012/009145. For example, the multi-block copolymers can be synthesized using linear polyethyleneimine (LPEI) blocks which have distinct patterns as compared to branched polyethyleneimines. Further, the composition or pharmaceutical composition can be made by the methods described in U.S. Pat. Appl. No. 2010/0004315 or U.S. Pat. Nos. 6,267,987 or 6,217,912.

U.S. Pat. Appl. No. 2010/0004313 describes how a formulation can include a nucleotide sequence and a poloxamer. Thus, the modified synthetic leptin mRNA of the invention can be used in a gene delivery composition with the poloxamer.

The modified synthetic leptin mRNA of the invention can be formulated with at least one degradable polyester which can contain polycationic side chains. Degradable polyesters include, but are not limited to, poly(serine ester), poly(L-lactide-co-L-lysine), poly(4-hydroxy-L-proline ester), and combinations thereof. In another embodiment, the degradable polyesters can include a PEG conjugation to form a PEGylated polymer.

Cationic Lipid Nanoparticles.

In some embodiments, the modified synthetic leptin mRNA of the invention can be encapsulated in a nanoparticle. Methods for nanoparticle packaging are well known in the art, and are described, for example, in Bose S et al. (2004) J. Virol. 78:8146; Dong Y et al. (2005) Biomaterials 26:6068; Lobenberg R et al. (1998) J Drug Target 5:171; Sakuma S R et al. (1999) Int. J. Pharm. 177:161; Virovic L et al. (2005) Expert Opin. Drug Deliv. 2:707; and Zimmermann E et al, (2001)Eur. J. Pharm. Biopharm. 52:203.

The use of cationic lipids for cellular delivery of nucleic acids has several advantages. The encapsulation of anionic compounds using cationic lipids is essentially quantitative due to electrostatic interaction. Cationic lipids may interact with the negatively charged cell membranes initiating cellular membrane transport. Akhtar et al. (1992) Trends Cell Biol. 2, 139; Xu et al. (1996) Biochemistry 35, 5616. Further, the molecular shape, conformation and properties of the cationic lipids may provide enhanced delivery efficiency from endosomal compartments to the cytosol. Semple S C et al. (2010) Nature Biotechnol. 28(2):172-176. Lipid nanoparticles containing cationic lipids have been shown to adequately encapsulate small inhibitory RNA (siRNA) and efficiently delivery siRNA to cells in animals. Jayaraman M et al (2012) Angew. Chemie Int. Ed. 51:1-6.

In one embodiment, the composition for encapsulating the modified synthetic leptin mRNA of the invention in a lipid nanoparticle comprises (i) a cationic lipid for encapsulation and for endosomal escape, (ii) a neutral lipid, for stabilization, (iii) a helper lipid, and (iv) a stealth lipid, which prevents aggregation.

“Cationic lipids” are positively charged lipids. For reviews of the use of cationic lipids to make cationic liposomes or cationic lipoplexes for nucleic acid delivery, see Gallas A et al. (2013) Chem. Soc. Rev. 42:7983-7997; Falsini et al. (2013) J. Med. Chem. dx.doi.org/10.1021/jm400791q; and other references provided herein. In specific embodiments, the cationic lipid can be selected from Cationic Lipid A, Cationic Lipid B, Cationic Lipid C and Cationic Lipid D, each of which is described herein.

“Neutral lipids” is an operational term for any lipid that is soluble only in solvents of very low polarity. Neutral lipids suitable for use in a delivery agent of the invention include a variety of neutral, uncharged or zwitterionic lipids, such as: 5-heptadecylbenzene-1,3-diol (resorcinol), dipalmitoylphosphatidylcholine (DPPC), distearoylphosphatidylcholine (DSPC), phosphocholine (DOPC), dimyristoylphosphatidylcholine (DMPC), phosphatidylcholine (PLPC), 1,2 distearoyl-sn-glycero 3 phosphocholine (DAPC), phosphatidylethanolamine (PE), egg phosphatidylcholine (EPC), dilauryloylphosphatidylcholine (DLPC), dimyristoylphosphatidylcholine (DMPC), 1 myristoyl 2 palmitoyl phosphatidylcholine (MPPC), 1 palmitoyl 2 myristoyl phosphatidylcholine (PMPC), 1-palmitoyl-2-stearoyl phosphatidylcholine (PSPC), 1,2-diarachidoyl-sn glycerol-3-phosphocholine (DBPC), 1-stearoyl-2-palmitoyl phosphatidylcholine (SPPC), 1,2-dieicosenoyl-sn-glycerol-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphophatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In one embodiment, the neutral phospholipid is selected from the group consisting of DSPC and DMPE.

“Helper lipids” are lipids that enhance transfection (e.g. transfection of the nanoparticle including the biologically active agent). The mechanism by which the helper lipid enhances transfection may include, e.g., enhancing particle stability or enhancing membrane fusogenicity. Helper lipids include steroids and alkyl resorcinols. Helper lipids suitable for use in a delivery agent of the invention include cholesterol, 5-heptadecylresorcinol and cholesterol hemisuccinate.

“Stealth lipids” are lipids that increase the length of time for which the nanoparticles can exist in vivo, e.g. in the blood. Stealth lipids suitable for use in a delivery agent of the invention include, but are not limited to, stealth lipids having a hydrophilic head group linked to a lipid moiety. Examples of such stealth lipids include compounds described in Intl. Pat. Appl. No. WO 2011/076807. Other stealth lipids suitable for use in a delivery agent of the invention and information about the biochemistry of such stealth lipids can be found in Romberg et al. (2008) Pharmaceutical Research 25(1): 55 71 and Hoekstra et al. (2004) Biochimica et Biophysica Acta 1660: 41-52. Suitable stealth lipids can be selected from among the molecules similar to poly(ethylene glycol) (PEG), including poly(ethylene oxide) and polymers based on poly(oxazoline), poly(vinyl alcohol), poly(glycerol), poly(N-vinylpyrrolidone), polyaminoacids and poly[N (2 hydroxypropyl) methacrylamide]. Additional suitable PEG lipids are disclosed in Intl. Pat. Appl. No. WO 2006/007712. PEG lipids include polyethyleneglycol diacylglycerol or polyethyleneglycol diacylglycamide (PEG DAG) conjugates including those comprising a dialkylglycerol or dialkylglycamide group having alkyl chain length independently comprising from about C4 to about C40 saturated or unsaturated carbon atoms. The dialkylglycerol or dialkylglycamide group can further comprise one or more substituted alkyl groups. In any of the examples described herein, the PEG conjugate can be selected from PEG dilaurylglycerol, PEG dimyristylglycerol (PEG-DMG) (catalog # GM 020 from NOF, Tokyo, Japan), PEG dipalmitoylglycerol, PEG disterylglycerol, PEG dilaurylglycamide, PEG dimyristylglycamide, PEG dipalmitoylglycamide, and PEG disterylglycamide, PEG cholesterol (1[8′(cholest 5 en 3[beta]oxy)carboxamido 3′,6′dioxaoctanyl]carbamoyl[omega]methyl poly(ethylene glycol), PEG DMB (3,4 ditetradecoxylbenzyl-[omega]-methyl poly(ethylene glycol) ether), 1,2 dimyristoyl-sn-glycerol-3-phosphoethanolamine N [methoxy(polyethylene glycol) 2000] (catalog #880150P from Avanti Polar Lipids, Alabaster Ala. USA).

In one embodiment, the stealth lipid is compound S024, which is described in Intl. Pat. Appl. No. WO 2011/076807. The chemical structure of compound S024 is:

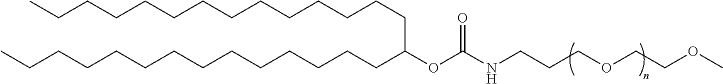

The chemical name of S024 is nonacosan-15-yl 2,5,8,11,14,17,20,23,26,29,32,35,38,41,44,47,50,53,56,59,62,65,68,70,72,75,78,81, 84,87,90,93,96,99,102,105,108,111,114,117,120,123,126,129,132,135-hexatetracontaoxatetracontahectan-140-ylcarbamate, where n=45.

In one embodiment, the delivery agent includes cholesterol as the helper lipid, a neutral lipid and a PEG lipid as a stealth lipid. In a more specific embodiment, the delivery agent comprises 30-60% cationic lipid selected from Cationic Lipid A, Cationic Lipid B, Cationic Lipid C or Cationic Lipid D, 5-10% helper lipid, 30-60% neutral lipid, and 1-5% PEG lipid. In an even more specific embodiment, the delivery agent comprises 30-60% cationic lipid selected from Cationic Lipid A, Cationic Lipid B or Cationic Lipid C, 5-10% helper lipid, 48% cholesterol, and 2% PEG lipid.

In one embodiment, the pH of the delivery agent is 4-6 at the time of encapsulation or formulation of the modified synthetic leptin mRNA. In a more specific embodiment, the pH of the delivery agent is 5-6 at the time of encapsulation or formulation. In a yet more specific embodiment, the pH of the delivery agent is 5.6-6.0 at the time of encapsulation or formulation. A suggested, although not required, pH for formulation of a lipid nanoparticle with Cationic Lipid C is pH 5.6. A suggested, although not required, pH for formulation of a lipid nanoparticle with Cationic Lipid B is pH 6.0.

Depending on the cationic lipid chosen, the percent mRNA encapsulation may be higher or lower at different pH values within this range. The optimal encapsulation pH for a particular cationic lipid formulation can be determined empirically within the range. The molar ratio of the four lipid components in the delivery agent and the cationic lipid amine to mRNA phosphate (N:P) molar ratio is generally useful guidance, but can be varied from and N:P ratio of 3:1 to an N:P ratio of 8:1. Variations in N:P ratio can affect mRNA encapsulation efficiency, particle analytics, and in vivo mRNA delivery performance. The ideal lipid formulation for in vivo mRNA delivery requirements can be determined empirically.

Delivery agents of the invention can be further optimized by one skilled in the art by adjusting the lipid molar ratio between these various types of lipids. In one embodiment, further optimization is obtained by adjusting one or more of: the desired particle size, N:P ratio, formulation methods.

In the improved processes described herein and in particular in EXAMPLE 34 and EXAMPLE 35, the modified synthetic leptin mRNA of the invention is formulated into cationic lipid nanoparticles using cationic lipids and rapid mixing. The encapsulation is efficient and the formulated particles can be suitable for delivery of the encapsulated mRNA to living cells in vitro and in vivo. TABLE 2 shows analysis results of leptin mRNA encapsulation in lipid nanoparticles by the two Improvement Processes described in EXAMPLE 34 and EXAMPLE 35.

TABLE 2
Results of Leptin mRNA Encapsulation in Cationic Lipid C Using
Improvement Process A and Improvement Process B

| | Cationic | Z-Ave | | | mRNA |
|---|---|---|---|---|---|
| Process | lipid | (nm) | PDI | Encapsulation | (μg/mL) |
| A (220 μg | Cationic | 107.2 | 0.145 | 94.7% | 47.4 |
| scale) | Lipid C | | | | |
| B (30 mg | Cationic | 112.0 | 0.120 | 95.6% | 1310 |
| scale) | Lipid C | | | | |

Pharmaceutical Compositions.

Formulations of the pharmaceutical compositions described herein can be prepared by any method known or hereafter developed in the art of pharmacology. In general, such preparatory methods include the step of bringing the active ingredient into association with an excipient and optionally one or more other accessory ingredients, and then optionally shaping or packaging the product into a desired single- or multi-dose unit.

Although the descriptions of pharmaceutical compositions provided herein are principally directed to pharmaceutical compositions which are suitable for administration to humans, it will be understood by the skilled artisan that such compositions are generally suitable for administration to animals of all sorts.

A pharmaceutical composition in accordance with the invention may be prepared, packaged or sold in bulk as a single unit dose or as a plurality of single unit doses. A “unit dose” is discrete amount of the pharmaceutical composition comprising a predetermined amount of the active ingredient. The amount of the active ingredient is generally equal to the dosage of the active ingredient which would be administered to a subject.

Pharmaceutical compositions may additionally contain a pharmaceutically acceptable excipient, which includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 22^{nd} edition (20012) Allen L V et al. eds., Pharmaceutical Press, discloses various excipients used in formulating pharmaceutical compositions and known techniques for their preparation.

In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia or the International Pharmacopoeia.

General considerations in the formulation or manufacture of pharmaceutical agents may be found, for example, in Remington's The Science and Practice of Pharmacy, 22^{nd} edition (20012) Allen L V et al. eds., Pharmaceutical Press.

The pharmaceutical compositions described herein can be characterized by one or more of the following properties:

Bioavailability.

The mRNA molecules, when formulated into a composition with a delivery agent as described herein, can exhibit an increase in bioavailability as compared to a composition lacking a delivery agent as described herein. The term “bioavailability” refers to the systemic availability of a given amount of an mRNA molecule administered to a mammal. Bioavailability can be assessed by measuring the area under the curve (AUC) or the maximum serum or plasma concentration (Cₘₐₓ) of the unchanged form of a compound following administration of the compound to a mammal. AUC is a determination of the area under the curve plotting the serum or plasma concentration of a compound along the ordinate (Y-axis) against time along the abscissa (X-axis). Generally, the AUC for a particular compound can be calculated using methods known to those of ordinary skill in the art and as described in G. S. Banker (1996) Modern Pharmaceutics, Drugs and the Pharmaceutical Sciences, v. 72, Marcel Dekker, New York, Inc., herein incorporated by reference.

The Cₘₐₓ value is the maximum concentration of the compound achieved in the serum or plasma of a mammal following administration of the compound to the mammal. The Cₘₐₓ value of a particular compound can be measured using methods known to those of ordinary skill in the art. The phrases “increasing bioavailability” or “improving the pharmacokinetics,” as used herein mean that the systemic availability of a first mRNA molecule, measured as AUC, Cₘₐₓ, or Cₘᵢₙ in a mammal is greater, when co-administered with a delivery agent as described herein, than when such co-administration does not take place.

Therapeutic Window.

The mRNA molecules, when formulated into a composition as described herein, can exhibit an increase in the therapeutic window of the administered mRNA molecule composition as compared to the therapeutic window of the administered mRNA molecule composition lacking a delivery agent as described herein. As used herein, “therapeutic window” refers to the range of plasma concentrations, or the range of levels of therapeutically active substance at the site of action, with a high probability of eliciting a therapeutic effect. In one embodiment, the therapeutic window for administration of the modified synthetic leptin mRNA formulation of the invention is an increase plasma leptin protein of 10 ng/mL above baseline, where baseline can be the plasma leptin in the subject before administration of the modified synthetic leptin mRNA formulation or can be the plasma leptin protein in comparable test patients.

Volume of Distribution.

The modified synthetic mRNA of the invention, when formulated into a composition as described herein, can exhibit an improved volume of distribution (V_{dist}). The V_{dist} relates the amount of the drug in the body to the concentration of the drug in the blood or plasma. The term “volume of distribution” refers to the fluid volume that would be required to contain the total amount of the drug in the body at the same concentration as in the blood or plasma: V_{dist} equals the amount of drug in the body/concentration of drug in blood or plasma. For example, for a 10 mg dose and a plasma concentration of 10 mg/L, the volume of distribution would be 1 liter. The volume of distribution reflects the extent to which the drug is present in the extravascular tissue. A large volume of distribution reflects the tendency of a compound to bind to the tissue components compared with plasma protein binding. In a clinical setting, V_{dist} can be used to determine a loading dose to achieve a steady state concentration. Based on pharmacokinetic studies presented in the EXAMPLES, V_{dist} is very low, meaning when compound leaves circulation compound does not reenter circulation.

### Dosages

Dosage and administration of the modified synthetic leptin mRNA formulation of the invention will vary with the condition to be treated and the therapeutic approach taken in a given instance. The dosages can also be different depending upon whether the modified synthetic leptin mRNA formulations are administered in in therapeutic or prophylactic amounts.

The dosages will also vary depending upon the approach taken, the mode of delivery and the disease to be treated. In one embodiment, dosages can be in the range of 0.2 mg ribonucleic acid polynucleotide/kg body weight (mg/kg, mpk). In another embodiment, dosages can be in the range of 0.6 mg ribonucleic acid polynucleotide/kg body weight (mg/kg, mpk). See, EXAMPLE 20 and EXAMPLE 23. In other embodiments, dosages can be up to 2 mg/kg or 4 mg/kg.

Therapeutic compositions containing at least one modified synthetic leptin mRNA of the invention can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired.

### Administration

A modified synthetic leptin mRNA formulation of the invention can be delivered to or administered to a subject by a variety of routes depending upon whether local or systemic treatment is desired and upon the area to be treated. Exemplary routes include intravenous and subcutaneous delivery routes.

Administration of a modified synthetic leptin mRNA of the invention can be provided by the subject or by another person, e.g., a caregiver. A caregiver can be any entity involved with providing care to the human, such as a hospital, a hospice, a doctors office, an outpatient clinic; a healthcare worker such as a doctor, nurse, or other practitioner; or a spouse, parent or guardian.

The modified synthetic leptin mRNA of the invention can be introduced into a cell in vivo or ex vivo by any manner that achieves intracellular delivery of the modified synthetic leptin mRNA, such that the translation and expression of the leptin polypeptide encoded by the modified synthetic leptin mRNA can occur in the cell. Absorption or uptake of a modified synthetic leptin mRNA of the invention by a cell can occur by unaided diffusive or active cellular processes or by auxiliary agents or devices described herein below or known in the art.

The modified synthetic leptin mRNA formulation of the invention or pharmaceutical composition including the modified synthetic leptin mRNA formulation of the invention may be administered in combination with one or more other therapeutic, prophylactic, diagnostic, or imaging agents. The phrase “in combination with,” does not mean that the agents must be administered at the same time or formulated for delivery together, although these methods of delivery are within the scope of the invention. The modified synthetic leptin mRNA formulation of the invention can be administered concurrently with, prior to, or subsequent to, one or more other desired therapeutics or medical procedures. In general, each agent will be administered at a dose or on a time schedule determined for that agent. In some embodiments, the invention encompasses the delivery of pharmaceutical, prophylactic, diagnostic, or imaging compositions in combination with agents that may improve their bioavailability, reduces or modify their metabolism, inhibit their excretion or modify their distribution within the body.

Devices and methods known in the art for administration to subject, organs, tissues or cells are contemplated for use in conjunction with the modified synthetic leptin mRNA formulations of the invention. These include, for example, methods and devices having needles, hybrid devices employing for example lumens or catheters.

In some embodiments, the modified synthetic leptin mRNA of the invention is introduced into a target cell by transfection, nucleofection, lipofection, electroporation (see, e.g., Wong and Neumann (1982) Biochem. Biophys. Res. Commun. 107:584-87), biolistics (i.e., particle bombardment), cell fusion or the like.

In some embodiments, the modified synthetic leptin mRNA formulation of the invention is administered in a single dose or in two or more doses. If desired to facilitate repeated or frequent infusions, a non-implantable delivery device, e.g., needle, syringe, pen device, or implantatable delivery device, e.g., a pump, semi-permanent stent (e.g., intravenous, intraperitoneal, intracisternal or intracapsular), or reservoir can be advisable. In some such embodiments, the delivery device can include a mechanism to dispense a unit dose of the pharmaceutical composition comprising a modified synthetic leptin mRNA of the invention. In other embodiments, a device releases the pharmaceutical composition comprising a modified synthetic leptin mRNA of the invention continuously, e.g., by diffusion. In some embodiments, the device can include a sensor that monitors a parameter within a subject. For example, the device can include pump, e.g., and, optionally, associated electronics. Exemplary devices include stents, catheters, pumps, artificial organs or organ components (e.g., artificial heart, a heart valve, etc.), and sutures.

A modified synthetic leptin mRNA of the invention can also be delivered through the use of implanted, indwelling catheters that provide a means for injecting small volumes of fluid containing the modified synthetic leptin mRNA of the invention directly into local tissues. The proximal end of these catheters can be connected to an implanted access port surgically affixed to the subject's body or to an implanted drug pump located in, for example, the subject's torso.

Alternatively, implantable delivery devices, such as an implantable pump can be employed. Examples of the delivery devices for use with the compositions comprising a modified synthetic leptin mRNA of the invention include the Model 8506 investigational device by Medtronic, Inc. of Minneapolis Minn., USA) which can be implanted subcutaneously in the body or on the cranium, and provides an access port through which therapeutic agents can be delivered. Alternatively, the modified synthetic leptin mRNA of the invention can be delivered by a wide variety of devices, such as those described by U.S. Pat. Nos. 5,735,814, 5,814,014, and 6,042,579. Using the teachings described herein, those of skill in the art will recognize that these and other devices and systems can be suitable for delivery of the modified synthetic leptin mRNA of the invention.

In some such embodiments, the delivery system further comprises implanting a pump outside the body, the pump coupled to a proximal end of the catheter, and operating the pump to deliver the predetermined dosage of a modified synthetic mRNA of the invention through the discharge portion of the catheter. A further embodiment comprises periodically refreshing a supply of the modified synthetic leptin mRNA of the invention to the pump outside the body.

A method for delivering therapeutic agents to a solid tissue has been described by Bahami et al. in U.S. Pat. Appl. No. 2011/0230839. An array of needles is incorporated into a device which delivers a substantially equal amount of fluid at any location in the solid tissue along each needle's length.

A device for delivery of biological material across the biological tissue has been described by Kodgule et al. in U.S. Pat. Appl. No. 2011/0172610. Multiple hollow micro-needles made of one or more metals and having outer diameters from about 200 microns to about 350 microns and lengths of at least 100 microns are incorporated into the device which delivers peptides, proteins, carbohydrates, nucleic acid molecules, lipids and other pharmaceutically active ingredients or combinations thereof.

A delivery probe for delivering a therapeutic agent to a tissue has been described by Gunday et al. in U.S. Pat. Appl. No. 2011/0270184. Multiple needles are incorporated into the device which moves the attached capsules between an activated position and an inactivated position to force the agent out of the capsules through the needles.

A multiple-injection medical apparatus has been described by Assaf in U.S. Pat. Appl. No. 2011/0218497. Multiple needles are incorporated into the device which has a chamber connected to one or more of the needles and a means for continuously refilling the chamber with the medical fluid after each injection.

A method for the transdermal delivery of a therapeutic effective amount of iron has been described by Berenson in U.S. Pat. Appl. No. 2010/0130910. Multiple needles may be used to create multiple micro channels in stratum corneum to enhance transdermal delivery of the ionic iron on an iontophoretic patch.

A method for delivery of biological material across the biological tissue has been described by Kodgule et al. in U.S. Pat. Appl. No. 2011/0196308. Multiple biodegradable microneedles containing a therapeutic active ingredient are incorporated in a device which delivers proteins, carbohydrates, nucleic acid molecules, lipids and other pharmaceutically active ingredients or combinations thereof.

A method for delivering genes, enzymes and biological agents to tissue cells has described by Desai in U.S. Pat. Appl. No. 2003/0073908. Multiple needles are incorporated into a device which is inserted into a body and delivers a medication fluid through the needles.

A micro-needle transdermal transport device has been described by Angel et al. in U.S. Pat. No. 7,364,568. Multiple needles are incorporated into the device which transports a substance into a body surface through the needles which are inserted into the surface from different directions.

A device for subcutaneous infusion has been described by Dalton et al. in U.S. Pat. No. 7,150,726. Multiple needles are incorporated into the device which delivers fluid through the needles into a subcutaneous tissue.

A device and a method for intradermal delivery of vaccines and gene therapeutic agents through microcannula have been described by Mikszta et al. in U.S. Pat. No. 7,473,247. At least one hollow micro-needle is incorporated into the device which delivers the vaccines to the subject's skin to a depth of between 0.025 mm and 2 mm.

A device for withdrawing or delivering a substance through the skin has been described by Down et al. in U.S. Pat. No. 6,607,513. Multiple skin penetrating members which are incorporated into the device have lengths of about 100 microns to about 2000 microns and are about 30 to 50 gauge.

A method for enhanced transport of drugs and biological molecules across tissue by improving the interaction between micro-needles and human skin has been described by Prausnitz et al. in U.S. Pat. No. 6,743,211. Multiple micro-needles are incorporated into a device which is able to present a more rigid and less deformable surface to which the micro-needles are applied.

A multiple needle holder and a subcutaneous multiple channel infusion port has been described by Brown in U.S. Pat. No. 4,695,273. Multiple needles on the needle holder are inserted through the septum of the infusion port and communicate with isolated chambers in the infusion port.

A dual hypodermic syringe has been described by Horn in U.S. Pat. No. 3,552,394. Two needles incorporated into the device are spaced apart less than 68 mm and may be of different styles and lengths, thus enabling injections to be made to different depths.

Methods and devices for administration of substances into at least two compartments in skin for systemic absorption and improved pharmacokinetics have been described by Pettis et al. in Intl. Pat. Appl. No. WO 2003/094995. Multiple needles having lengths between about 300 um and about 5 mm are incorporated into a device which delivers to intradermal and subcutaneous tissue compartments simultaneously.

A drug delivery device with needles and a roller has been described by Zimmerman et al. in Intl. Pat. Appl. No. WO 2012/006259. Multiple hollow needles positioned in a roller are incorporated into the device which delivers the content in a reservoir through the needles as the roller rotates.

A method for administering multiple-component therapies has been described by Nayak in U.S. Pat. No. 7,699,803. Multiple injection cannulas can be incorporated into a device wherein depth slots may be included for controlling the depth at which the therapeutic substance is delivered within the tissue.

A device and a method for delivering fluid into a flexible biological barrier have been described by Yeshurun et al. in U.S. Pat. Nos. 7,998,119 and 8,007,466, respectively. The micro-needles on the device penetrate and extend into the flexible biological barrier and fluid is injected through the bore of the hollow micro-needles.

If so desired, a mammal or subject can be pre-treated with an agent (e.g., a homing factor). For example, a homing factor can be administered to enhance cell targeting to a tissue and can be placed at a site to encourage cells to target the desired tissue. Direct injection of homing factors into a tissue can be performed prior to systemic delivery of ligand-targeted cells.

### Assessment of Efficacy

The success of the administration of the modified synthetic leptin mRNA of the invention can be evaluated by the ordinarily skilled clinician, by monitoring one or more symptoms or markers of the condition being treated, including such conditions as congenital leptin deficiency, lipodystrophy or other condition where circulating leptin level is low. An assessment of efficacy includes any statistically significant improvement in one or more indicia of the condition. Where appropriate, a clinically accepted grade or scaling system for the given condition can be applied, with an improvement in the scale or grade being indicative of effective treatment.

Guidance for assessing the efficacy of the administration of the modified synthetic leptin mRNA of the invention can be provided by the results from the administration of other leptin therapies.

Licinio J et al. (2004) reported improvements in several therapeutic indices in leptin-deficient obese adults following subcutaneous treatment with metreleptin. Licinio J et al. (2004) reported increased physical activity, resolution of both type 2 diabetes and hypogonadism, and a reduction in body mass index. The patients' mean body mass index dropped to about 36.5 kg/m² after 6 months of treatment. The patients' mean body mass index dropped to about 28.9 kg/m² after 12 months of treatment. The patients' mean body mass index dropped to about 26.9 kg/m² after 18 months of treatment. Also, the patients' mean daily caloric intake dropped 49% after 2 weeks of treatment. The patients' serum leptin levels increased to about 12.67 ng/mL after 6 months of treatment. The patients' plasma lutenizing hormone (LH) levels increased to about 2.75 milliunits/mL after 6 months of treatment. During the 18 month course of treatment, triglyceride levels dropped at least 49%.

Ebihara K. et al. (2007) treated patients with generalized lipodystrophy were treated with metreleptin subcutaneously with metreleptin twice per day. The patients with generalized lipodystrophy showed significant improvement in fasting plasma glucose and serum triglyceride within 1 week of treatment. In patients with generalized or partial lipodystrophy, metreleptin treatment for four months significantly improved fasting glucose from 184±91 mg/dL to 146±14 mg/dL hemoglobin A1c from 8.5%±2.1% to 7.3%±0.3% and triglycerides from 479±80 mg/dL to 254±40 mg/dL. Ebihara et al. (2007) also showed fasting glucose levels reduced from about 172 to about 120 mg/dL, and triglyceride levels reduced from about 700 to about 260 mg/dL within 1 week of treatment.

Chan J L et al. (2011) reported reductions in several therapeutic indices during a 3-year metreleptin treatment period. Plasma levels of hemoglobin A1c (initially elevated at about 8.5%) were reduced by about 2.1%. Hemoglobin A1c (HbA1c) is a glycated hemoglobin and is expressed as a percentage of total hemoglobin, such that a higher percentage reflects a higher levels of glucose in the circulation over time. Plasma levels of triglycerides (initially elevated at about 479 mg/dL) were reduced by about 35.4%. After three years of metreleptin treatment, fasting glucose levels went from about 184 mg/dL to about 124 mg/dL, hemoglobin A1c levels went from about 8.5% to about 6.0% and triglycerides went from about 743 mg/dL to about 164 mg/dL. Chan et al. (2011) referenced Oral E A et al. (2002) N. Engl. J. Med. 346(8):570-8, which describes results methods of testing leptin, glucose, triglycerides and glycosylated hemoglobin following leptin-replacement therapy for lipodystrophy. The methods of testing described by Oral et al. (2002) and others provide guidance for relevant testing of blood, plasma or serum.

Guidance for administration of the leptin mRNA formulation of the invention can be provided by the results provided by Licinio et al. (2004), Ebihara et al. (2007), Chan et al. (2011) and Oral et al. (2002). Because administration of the leptin mRNA of the invention delivers a leptin protein at least as effectively as the administration of metreleptin, there will be a comparable reduction in plasma glucose levels or triglyceride levels as after seven days of leptin treatment. Because administration of patients receiving metreleptin subcutaneously for seven days resulted in reduction of glucose and triglyceride levels, the administration of the leptin mRNA formulation of the invention results in a decrease of at least 30% in plasma glucose levels and at least 40% in plasma triglyceride levels.

Additional guidance for the reduction of plasma glucose levels and plasma triglyceride levels is found in EXAMPLE 37 and EXAMPLE 38.

All patents, oligonucleotide sequences identified by gene identification numbers, and other publications identified herein are expressly incorporated by reference for the purpose of describing and disclosing. This includes the methodologies described in the patents and publications that can be used in connection with the invention. All statements as to the date or representation as to the contents of these documents is based on the information available and does not constitute any admission as to the correctness of the dates or contents of these documents. In cases of conflicting statements between the cited source and the disclosure herein, the statement in the disclosure herein shall control.

Articles such as “a,” “an,” and “the” may mean one or more than one unless indicated to the contrary or otherwise evident from the context. As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

Where ranges are given, endpoints are included. Furthermore, values that are expressed as ranges can assume any specific value or subrange within the stated ranges in different embodiments of the invention, to the tenth of the unit of the lower limit of the range, unless the context clearly dictates otherwise.

The EXAMPLES, which follow, are illustrative of specific embodiments of the invention, and various uses thereof. They are set forth for explanatory purposes only, and are not to be taken as limiting the invention.

### EXAMPLES

### Example 1

### In Vivo Pharmacokinetic and Efficacy Study of Leptin DNA

The purpose of this EXAMPLE was to confirm that expression from a leptin-encoding polynucleotide construct could deliver human leptin protein. To determine the leptin protein expression from human leptin DNA and the in vivo efficacy, a hydrodynamic gene delivery method (HDI) was developed and employed. The delivery of naked DNA induced expression of leptin protein was followed by in vivo efficacy in ob/ob mice. The following materials were used:

TABLE 3
Materials for Efficacy Study of Leptin DNA

| Reagent | Vendor | Catalog # |
|---|---|---|
| 1x PBS | Ambion | AM9624 |
| 27G butterfly needle | TERUMO | SV*27EL |
| 27G needle | Becton Dickinson | 305109 |
| 3cc Syringe | Becton Dickinson | 309657 |
| DNA Vector control | LakePharma | pLEV113 |
| Human leptin DNA | LakePharma | pLEV113 containing SEQ ID |
| construct | | NO: 2 (SEQ ID NO: 14) |
| Saline Injectable | B. Braun | S4002-15SS |

Prior to HDI, mouse body weights and food weight were recorded. The mice were grouped according to their body weights.

Mice were prepared for injection by warming them under a heating lamp for ˜2 minutes, with the mice about 12 inches from heat lamp.

For the hydrodynamic injection procedure, the mice were placed in a restrainer and their tails cleaned with 70% alcohol. A 27 gauge butterfly needle connected with a 3 ml syringe was inserted into the tail vein, with bevel facing up, and the syringe plunger was pulled backwards to ensure blood is drawn into the syringe. The desired volume (7% of body weight, capped at 3.2 ml) of DNA constructs was injected over a short period (5-10 seconds) by hand. The needle was then withdrawn and bleeding stopped by adding pressure to the injection site with gauze.

For post-injection care, the mice were placed on a heating pad for a full recovery (˜15 min) under observation, and then put back in their housing room. The mice were then monitored twice daily in the first 48 hours.

Eight-nine week old, male, single housed ob/ob mice were used for the in vivo study. The human leptin DNA constructs described in TABLE 1 were diluted in injectable saline at doses of 30 μg, 3 μg, 0.3 μg and 0.03 μg per average group body weight.

On day 0, the animals were weighed and sorted according to average body weight. According to body weight, mice were injected in the tail vein at the above dose of DNA, which was then diluted into injectable saline. The final dose volume was 7% of the mouse body weight, with a cap of 3.0 ml. Mice were dosed. Then, body weight, health, and food intake (FI) were recorded on each of days 1, 2, 4, 7, 14, and 21. Additionally, on days 4 and 7, 15 μl of plasma was collected in order to assess leptin protein expression level.

Human leptin administered according to the procedure of this EXAMPLE decreased body weight (TABLE 4) and reduced food intake (TABLE 5) in leptin-deficient ob/ob mice after hydrodynamic injection of leptin DNA. Body fat analysis confirmed that body weight loss was completely due to loss in body fat.

TABLE 4
Effect of Human Leptin Expression Plasmid DNA on Body Weight
in ob/ob Mice Administered by Hydrodynamic Injection

| | | Leptin | Leptin | Leptin | Leptin | Leptin |
|---|---|---|---|---|---|---|
| Day | Vector | 0.003 μg | 0.03 μg | 0.3 μg | 3 μg | 30 μg |
| 1 | −1.0 | −1.8 | −1.1 | −1.2 | −0.7 | −1.7 |
| 2 | −0.7 | −0.9 | −1.5 | −1.5 | −1.8 | −3.0 |
| 4 | −0.6 | −1.6 | −2.7 | −3.7 | −4.2 | −5.9 |
| 7 | 0.2 | −0.8 | −2.0 | −5.9 | −7.5 | −9.9 |
| 14 | 2.5 | 1.7 | 0.6 | −10.3 | −13.6 | −18.4 |
| 22 | 3.9 | 3.4 | 1.8 | −13.8 | −17.6 | −22.3 |

Change in body weight in grams from baseline (day 0)

TABLE 5
Effect of human leptin expression plasmid DNA on food intake
in ob/ob mice administered by hydrodynamic injection

| | | Leptin | Leptin | Leptin | Leptin | Leptin |
|---|---|---|---|---|---|---|
| Day | Vector | 0.003 μg | 0.03 μg | 0.3 μg | 3 μg | 30 μg |
| 1 | 3.0 | 1.4 | 1.6 | 2.3 | 1.3 | 0.6 |
| 2 | 5.7 | 4.6 | 4.2 | 3.9 | 2.4 | 1.2 |
| 4 | 7.2 | 5.9 | 4.7 | 2.7 | 2.0 | 0.7 |
| 7 | 7.2 | 6.0 | 5.7 | 2.0 | 1.3 | 0.6 |
| 14 | 7.1 | 6.4 | 6.0 | 1.8 | 1.1 | 0.5 |
| 22 | 6.1 | 5.8 | 5.1 | 2.1 | 1.9 | 1.8 |

Food intake in grams per day

Human leptin protein ELISA assay. Human leptin in mouse plasma was measured by ELISA. Antibodies purchased from the R&D systems duoset (Cat# DY398E, part#840279 for capture antibody and part#840280 for detection antibody) were reconstituted using PBS and titered, again using PBS. The capture antibody was coated at 4 μg/mL in 30 μl/well on a white Nunc® Maxisorp 384 well plate (Cat#460372). After an overnight incubation at room temperature the capture antibody was aspirated and the plate blocked for 2 hours at room temperature with 90 μL/well of KPL milk blocker (Cat#50-82-00). Once the incubation was completed the plate was aspirated and recombinant standards and samples were added to the plate at 30 μL/well for 2 hours at 37° C. while shaking at 600 rpm. Sample/standard dilutions were made using casein sample diluent. Washing/aspiration 3 times with 100 μl/well followed, using Teknova plate wash solution (Cat# P1192). Next, detection antibody was diluted using casein detection antibody diluent to 12.5 ng/mL and added at 30 μl/well for 2 hours room temperature. After this incubation, the plate was washed again and a solution of poly-streptavidin-HRP (Cat#21140) at a 1:1250 dilution in HRP dilution buffer was added to each well (30 μL/well) and incubated for 30 minutes room temperature. A final wash/aspiration removed the HRP solution and a chemiluminescent substrate was added at 30 μL/well (Cat#1859678 & 1859679). The plate was quickly read using a SpectramaxM5 plate reader with a 50 ms integration time. The dynamic range of the ELISA is from 100-2,000 μg/mL of human leptin. The assay is applicable to plasma from mice, rats and cynomolgus monkeys. Plasma human leptin levels are shown in TABLE 6.

TABLE 6
Effect of Human Leptin Expression Plasmid DNA Administered
by Hydrodynamic Injection on Plasma Human
Leptin Protein Levels in ob/ob Mice

| | | Leptin | Leptin | Leptin | Leptin | Leptin |
|---|---|---|---|---|---|---|
| Day | Vector | 0.003 μg | 0.03 μg | 0.3 μg | 3 μg | 30 μg |
| 4 | 284 | BLQ | 1302 | 9470 | 112500 | 351541 |
| 7 | 256 | 281 | 602 | 5211 | 72546 | 150666 |

Human leptin in pg/mL, BLQ = below limit of quantification

Pharmacokinetic (PK) and pharmacodynamic (PD) analyses established a leptin EC₅₀ of 1.4 ng/mL (85 pM) for the suppression of food intake.

### Example 2

### In Vitro Transcription and Capping of Modified Synthetic Leptin mRNA

The modified synthetic leptin mRNA of this EXAMPLE was generated by in vitro transcription (IVT), purified by lithium chloride (LiCl) purification, and then capped using a commercially available kit from New England Biolabs (Beverly Mass. USA).

Materials and reagents are shown in TABLE 7.

TABLE 7
Materials and Reagents for In vitro Transcription and Capping

| Reagent | Vendor | Catalog # | Notes |
|---|---|---|---|
| Nuclease-free | | | |
| water | | | |
| Tris-HCl pH 8.0 | Life Technologies | AM9855G | |
| MgCl₂ | Life Technologies | AM9530G | |
| ATP, CTP, GTP, | New England | N0450L | |
| UTP | Biolabs | | |
| Pseudouridine (Ψ) | TriLink Biotech | N-1019 | |
| DTT | Sigma | 43816 | |
| Spermidine | Sigma | 85558 | Stock provided at |
| | | | ~6.37M. Dilute to |
| | | | 1M and store in |
| | | | single use aliquots |
| | | | at −80° C. |
| Linearized plasmid | | | SEQ ID NO: 14 |
| DNA | | | |
| Pyrophosphatase | New England | M2403L | |
| | Biolabs | | |
| RNase inhibitor | New England | M0307L | |
| | Biolabs | | |
| T7 RNA | New England | M0251L | |
| polymerase | Biolabs | | |
| DNase | New England | M0303 | |
| | Biolabs | | |
| LiCl | Life Technologies | AM9480 | |
| Vaccinia capping | New England | M2080S | |
| system | Biolabs | | |
| mRNA cap 2′-O- | New England | M0366S | |
| methyltransferase | Biolabs | | |

Transcription reactions are assembled as listed in TABLE 8, with care towards the use of RNase-free tubes, tips and practices.

TABLE 8
In vitro Transcription Reaction

| Reagent | Concentration | Notes |
|---|---|---|
| Nuclease-free | Remaining | |
| water | volume | |
| Tris-HCl pH 8.0 | 40 | |
| (mM) | | |
| MgCl₂ (mM) | 20 | |
| ATP, CTP, GTP, | 4 | |
| UTP (mM) | | |
| Pseudouridine | 4 | To make 100% pseudouridine |
| (mM) | | mRNA, do not include UTP in |
| | | reaction. To make 100% |
| | | unmodified mRNA, do not |
| | | include pseudouridine in reaction |
| DTT (mM) | 10 | |
| Spermidine (mM) | 2 | Dilute 1M stock 1:10 in water |
| Linearized plasmid | 0.05 | SEQ ID NO: 14 |
| DNA (μg/μL) | | |
| Pyrophosphatase | 0.004 | |
| (U/μL) | | |
| RNase inhibitor | 1 | |
| (U/μL) | | |
| T7 RNA | 5 | |
| polymerase (U/μl) | | |

The procedure in this EXAMPLE for making modified synthetic leptin mRNA is as follows:
1. Incubate the materials above for 2 hours at 30° C., while monitoring the temperature. Digest the DNA template by adding 0.04 U/μL DNase, and then incubate for 30 minutes at 37° C.
2. Add LiCl to a final concentration of 2.81 M, mixing well and incubating for over an hour at −20° C. Centrifuge the mixture at 4° C. for 15 minutes at a maximum speed of approximately 20,000×g (max speed). Remove the supernatant and wash the pellet with 1 mL 70% ethanol. Centrifuge as described immediately above for 10 minutes, then remove the supernatant and centrifuge again as described above for less than one minute.
3. Remove the remaining ethanol and resuspend the pellet in nuclease-free water. Observe dissolving of pellet and pipet up and down and incubate at 37° C. to help the pellet go into solution. Measure the concentration and adjust to ˜1 μg/μL.
4. Add 10% volume of 3M sodium acetate pH 5.5 and mix well. Add 1 volume of room temperature isopropanol and mix well. Incubate overnight at −20° C. Centrifuge the mixture at 4° C. for 15 minutes at a maximum speed of approximately 20,000×g (max speed). Remove supernatant and wash pellet with 1 mL 70% ethanol. Centrifuge as described immediately above for 10 minutes, then remove the supernatant and centrifuge again as described above for less than one minute.
5. Remove the remaining ethanol and resuspend the pellet in nuclease-free water. Carefully observe dissolving of pellet and pipet up and down and incubate at 37° C. to help the pellet go into solution. Measure concentration and adjust to ˜4 μg/μL.

The modified synthetic leptin mRNA can then be stored at −80° C. until capping, and the concentration measured again upon thawing.

For capping, the procedure used was that of New England BioLabs. The synthetic leptin mRNA and water mixture is heat denatured at 65° C. for 10 minutes, and then transferred to cold block to quench for 5 minutes. The stock solution of S-adenosyl methionine (SAM) (32 mM) is diluted 1:8 in water to 4 mM immediately before use, then the remaining reaction components are added in the order specified in TABLE 9.

TABLE 9
Capping Reaction

| | Stock | |
|---|---|---|
| Reagent | concentration | Final concentration |
| mRNA (μg/μl) | | 0.5 |
| Water | | Remaining volume |
| 10x capping buffer (x) | 10x | 1x |
| GTP (mM) | 10 | 0.5 |
| SAM (mM) | 4 | 0.2 |
| RNase Inhibitor (U/μL) | 40 | 1 |
| Vaccinia capping enzyme (U/μL) | 10 | 0.5 |
| mRNA Cap 2′-O- | 50 | 2.5 |
| Methyltransferase (U/μL) | | |

Then, incubate the mixture for one hour at 37° C. The sample is purified by LiCl precipitation as described above, and then stored at −80° C.

### Example 3

### FPLC Purification of Modified Synthetic Leptin mRNA

The modified synthetic mRNA of the invention was purified on a Fast Protein Liquid Chromatography (FPLC) column (Semi-Prep RNASep 21×100 mm column (Transgenomic Inc. catalog # RPC-99-2110)). The columns were housed in a column heater (Timberline Instruments (catalog # TL-105)) with the temperature set to 50° C., and were pre-equilibrated with 5 column volumes of 62% of Wave optimized Buffer A (0.1 M Triethylammonium acetate, pH 7.0) (Transgenomic Inc. catalog #553401)) and 38% Wave optimized Buffer B (0.1 M Triethylammonium acetate, pH 7.0, 25% acetonitrile) (Transgenomic Inc. catalog #553402). One column volume is equal to 35 mL.

1.5 mL of each modified synthetic mRNA solution was injected into 2 mL sample loop. Then, the FPLC run was begun according to the gradient described in TABLE 10. The flow rate was set at 5 mL/min.

TABLE 10
FPLC Gradient

| Number of column | |
|---|---|
| volumes | Notes |
| 2 | Start fraction collection. Wash column with 38% |
| | Buffer B. |
| 0.5 | Linear gradient, increase % Buffer B from 38 to 45 |
| 2 | Linear gradient, increase % Buffer B from 45 to 65 |
| 2 | End fraction collection immediately. Hold at 65%. |

The fractions were pooled, and the modified synthetic mRNA was isolated by isopropanol and sodium acetate precipitation. The fractions were pooled correlating to the first mRNA peak (eluting ∞23 minutes post injection).

The sample was concentrated with an Amicon Ultra-15 Centrifugal Filter unit, 30K (Millipore catalog # UFC 903024), at 22° C., for 15 minutes at 4750 rpm, and buffer was exchanged to water by diluting the concentrate with water and putting the mixture through the centrifuge two times.

The concentrated mRNA sample was transferred to an Eppendorf tube and the mRNA was precipitated with 1/10 of volume 3 M sodium acetate, pH 5.5 (Ambion # AM9740) and 2× volume 2-proponol, and 1/100 volume GlycoBlue (Ambion catalog # AM9515), and kept at −20° C. overnight.

The mixture was centrifuge at 4° C. for 15 min. The supernatant was removed, and the pellet washed with 1 mL of cold 70% ethanol (Sigma catalog #459844). The mixture was centrifuged for 3 minutes, and the supernatant removed.

The pellet was resuspend in nuclease-free water (Life Technologies catalog #10977-015). Pipetting and incubation at 37° C. for approximately 5-10 minutes helped the pellet go into solution. The concentration was measured and adjusted to −1.5 mg/mL. The mRNA was then stored at −80° C. until capping, and concentration was measured again upon thawing.

### Example 4

### mRNA Purification by HPLC

The modified synthetic mRNA of the invention was purified on by High Performance Liquid Chromatography. A Shimadzu SCL-10A VP system controller was used to synchronize separation to a SIL-10AP autosampler, LC-8A liquid chromatograph, SPD-20A UV/Vis detector and an FRC-10A fraction collector. A Phenomenex Luna C18(2) 30×100 mm 5μ 100A reversed phase HPLC column was used for chromatographic separation of hLeptin mRNA. The column was housed in a Timberline Instruments T105 dual column and mobile phase heating unit. Reagents are listed in TABLE 11.

TABLE 11
Reagents for Leptin mRNA Purification by HPLC

| Product | Vendor | Catalogue number |
|---|---|---|
| Chromasolv HPLC water | Sigma Aldrich | 270733 |
| Chromasolv HPLC acetonitrile | Sigma Aldrich | 34998 |
| Isopropanol | Sigma Aldrich | 34965 |
| RNase free water | Invitrogen | 10977-015 |
| Sodium Hydroxide | Sigma Aldrich | S5881 |
| Acetic Acid >99% | Sigma Aldrich | A6283 |
| 1M TEAA | Sigma Aldrich | 90358 |
| Pellicon3 88 cm² cassette | Millipore | P3B030A00 |

100 mg of mRNA at a concentration of 4 mg/mL in 0.1 M TEAA and 4.5% acetonitrile was loaded onto the column by 6-8 injections using a 5 ml loop. Mobile phase A was 0.1 M TEAA in water and mobile phase B was 0.1 M TEAA in 90:10 acetonitrile:water. The flow rate was held constant at 10 ml/min for the full gradient and both the HPLC column and mobile phases were maintained at 65° C. at all times. The time program for the HPLC run is shown in TABLE 12. Human leptin mRNA was collected in 12 ml fractions once the 260 nm wavelength reading reached 20 mV. Sample collection continued until the tailing end of the HPLC peak was under 20 mV.

TABLE 12
Time Program for Purification of Leptin mRNA by HPLC

| Time (minutes) | % Buffer B | % organic |
|---|---|---|
| 0 | 5 | 4.5 |
| 10 | 5 | 4.5 |
| 36 | 18 | 16.2 |
| 38 | 27.8 | 25 |
| 43 | 27.8 | 25 |
| 43.5 | 5 | 4.5 |
| 56 | 5 | 4.5 |

HPLC fractions were buffer exchanged into water prior to any downstream test and application. Buffer exchange was performed on a Millipore Cogent pScale TFF with three Pellicon3 88 cm² cassettes. Individual LC fractions or combined fractions were poured into the unit's sample chamber and exchanged at least 100× into pyrogen and RNase-free water. The final volume of the sample in water was optimized to ensure that the final concentration of human leptin mRNA was at least 1 mg/mL. Human leptin mRNA samples in water were stored at −80° C. until capping, and concentration was measured again upon thawing when needed for in vivo experiments.

### Example 5

### Packaging of Modified Synthetic Leptin mRNA in a Delivery Vehicle

The materials in this EXAMPLE used were as follows:

TABLE 13
Materials for Packaging of Modified Synthetic Leptin mRNA

| Item | Vendor | Catalog # |
|---|---|---|
| Cationic lipid | Novartis | Selected from |
| | | Cationic Lipid |
| | | A, Cationic |
| | | Lipid B or |
| | | Cationic Lipid C |
| 1,2-distearoyl-sn-glycero-3- | Corden | LP-R4-076 |
| phosphocholine (DSPC) | | |
| Cholesterol | Sigma | C8667 |
| Lipidated Polyethylene Glycol | Novartis | S024 |
| (PEG lipid) | | |
| Ethanol | Sigma | 459844 |
| Nuclease-free water | Life Technologies | 10977 |
| 100 mM citrate buffer, pH 6.0 | Teknova | Q2446 |
| Amicon Ultra-15 Centrifugal | Millipore | UFC903024 |
| Filter unit, 30K MWCO | | |
| RNaseZap | Life Technologies | AM9780 |
| Syringe Pump | KD Scientific | KDS220 |
| 10X PBS | Lonza | S1226 |
| SnakeSkin dialysis tubing | Thermo Scientific | 68100 |
| 10,000 MWCO | | |
| Minimate TFF system, 110 V | PALL Corporation | OAPMP110 |
| Minimate tangential flow | PALL Corporation | OA500C12 |
| filtration capsule, Omega | | |
| 500K membrane | | |
| Quant-iT Ribogreen RNA | Life Technologies | R11490 |
| Assay Kit | | |
| TE buffer | Promega | V6231 |
| Triton X-100 | Sigma | T8787 |
| Zetasizer Nano ZS | Malvern | ZEN3600 |

Modified synthetic mRNAs of the invention were packaged into lipid nanoparticles at a cationic lipid amine group to mRNA phosphate group (N:P) molar ratio=4:1, dialyzed, and concentrated. As an example, amounts are shown for the protocol resulting in ˜2 mg packaged modified synthetic mRNA in a concentration of >0.4 mg/mL mRNA.

Using RNase-free reagents, tubes, tips, and practices, the lipid nanoparticle mixture reagents were weighed and mixed in a vial as described in TABLE 14.

TABLE 14
Lipid Nanoparticle Mixture

| Reagent | Final concentration (mM) | mg |
|---|---|---|
| Cationic lipid | 6 | 39.92 |
| DSPC | 1.5 | 10.43 |
| Cholesterol | 7.2 | 24.50 |
| PEG lipid | 0.3 | 7.13 |

Ethanol (8.8 mL) was added to the lipids, representing a 1.1× ratio of the volume needed, for ease of processing. The mixture was briefly sonicated and gently agitated for 5 minutes at 37° C. Subsequently, the mixture was incubated without agitation at 37° C. until ready for use.

The modified synthetic mRNA was exchanged from water into pH 6.0 buffer by loading mRNA solution onto Amicon Ultra-15 centrifugal device, and centrifuging for 15 minutes at 4,000 rpm at 4° C. The concentrated mRNA was resuspended in pH 6.0 citrate buffer and the mRNA concentration was measured.

The final modified synthetic mRNA concentration of 0.5 mg/mL in pH 6.0 citrate buffer was prepared in a rinsed scintillation vial (4 mg mRNA in 8 mL), and the final concentration of the mRNA solution was measured. The mRNA dilution was incubated at 37° C. until ready for use.

Three 10 ml syringes were prepared, with 8 mL of each: (a) lipid mixture; (b) mRNA solution; (c) citrate buffer. Syringes (a) and (b) were attached to the Leur fittings of the T-shaped junction. Briefly, a P727 T-mixer with 0.5 mm inner diameter attached to P652 adaptors (IDEX, Oak Harbor Wash. USA). Syringes (a) and (b) were attached to P658 Luer fittings (IDEX). The syringes (a) and (b) were connected to the T-mixer by PTFE 0.8 mm inner diameter tubing (#3200068, Dolomite, Royston, UK) with P938x nuts and ferrules (IDEX). Syringe (c) was attached to a Luer fitting connected to a final single tubing by P938x a nut and ferrule. The ends of the tubing were secured together over pre-rinsed beaker with stir bar and gently stirred.

The syringe pump settings were set to appropriate syringe manufacturer and size, and a volume (8 mL) and flow rate of 1.0 mL/min were entered. The pump was started, and the resulting material collected into RNase-free 50 mL plastic beaker with a stir bar. The suspension of lipid nanoparticles containing mRNA was transferred to dialysis tubing, 2-3 mL per bag and dialyzed into phosphate-buffered saline (PBS) at 4° C. overnight.

The divided material was pooled into one 15 mL conical tube. The lipid nanoparticle (LNP) suspension was concentrated using tangential flow filtration (TFF). Using fresh tubing to connect fresh 500K molecular weight cut-off capsule to the Minimate system, the TFF system was prepared by rinsing with 500 mL RNA-free water at a flow rate of 150 rpm.

The lipid nanoparticle/modified synthetic mRNA suspension was loaded into TFF unit reservoir and concentrated at a flow rate of 75 mL/min to 2-3 ml final volume.

The percent encapsulation of modified synthetic mRNA was determined using Quant-iT Ribogreen RNA Assay kit from Life Technologies (Grand Island N.Y. USA). The lipid nanoparticle/modified synthetic mRNA suspension was assayed by fluorescence measurement in buffer (mRNA outside the particle) and in buffer plus detergent (total mRNA). A 1000 ng/mL stock from the provided ribosomal RNA was prepared, and stock used stock to generate a standard curve for the Ribogreen assay, following the ratio of TE and TE+0.75% Triton X-100 shown in TABLE 15. For the assay, samples are prepared in TE buffer or TE plus Triton and the fluorescent reagent is added to each. The difference calculated is the mRNA inside the particle.

TABLE 15
Standard Curve (Preparation for Duplicate Samples)

| RNA concentration | Volume 1000 ng/mL | |
|---|---|---|
| (ng/mL) | stock (μL) | Volume buffer (μL) |
| 0 | 0 | 250 |
| 20 | 5 | 245 |
| 100 | 25 | 225 |
| 500 | 125 | 125 |
| 1000 | 250 | 0 |

Samples were prepared in TE buffer and TE buffer+0.75% Triton X-100 with appropriate dilution so that reading is in the standard curve (400-600 fold). 100 μL standard/sample were added per well in a 96-well plate. The Ribogreen reagent was diluted 1:200 in TE buffer and 100 μL was added to each well.

The sample fluorescence was measured using a fluorescence microplate reader, excitation at 480 nm, emission at 520 nm. The fluorescence value of the reagent blank was subtracted from the fluorescence value for each RNA sample to generate a standard curve of fluorescence versus RNA concentration. The fluorescence value of the reagent blank was subtracted from that of each of the samples and the RNA concentration of the sample from the standard curve was determined. The percent encapsulation of the sample was determined by dividing the difference in concentrations between sample plus Triton and just sample by the sample plus Triton concentration. A 6-fold dilution of the lipid nanoparticle/modified synthetic suspension was made, and the diameter and polydispersity index determined using a Zetasizer Nano ZS instrument (Malvern Instruments, Ltd, Worcestershire, UK).

### Example 6

### Leptin mRNA Packaged in Cationic Lipid A

### In Vivo Pharmacokinetic Study

Leptin protein expression was induced following intravenous and subcutaneous delivery of modified synthetic leptin mRNA of the invention. The expression of leptin protein (as measured by the ELISA described in EXAMPLE 1) led to in vivo efficacy in leptin deficient ob/ob mice.

The materials used for administration of the modified synthetic mRNA in Cationic Lipid A to mice are detailed in TABLE 16.

TABLE 16
Materials Used for Administration

| Reagent | Vendor | Catalog # |
|---|---|---|
| 1x PBS | Ambion | AM9624 |
| 27G needle | Becton Dickinson | 305109 |
| 1cc Syringe | Becton Dickinson | 309659 |
| 3cc Syringe | Becton Dickinson | 309657 |
| Saline Injectable | B. Braun | S4002-15SS |

### Example 7

### Mouse Intravenous Tail Vein Injection of Modified Synthetic Leptin mRNA

Before the tail vein injection, mouse body weights were recorded and diet weighted, with mice grouped according to their body weights. Mice were prepared by warming them under a heating lamp for ˜2 minutes, with the mice about 12 inches from heat lamp.

For the tail vein injection procedure, the mice were placed in a restrainer and their tails cleaned with 70% alcohol. A 27 gauge needle (Becton Dickinson, Catalogue #305109) connected with a 1 ml syringe (Becton Dickinson, Catalogue #309659) was inserted into the tail vein, with bevel facing up, and the syringe plunger was pulled backwards to ensure blood is drawn into the syringe. The desired volume of modified synthetic leptin mRNA was injected by hand with moderate pressure and speed. The needle was then withdrawn and bleeding stopped by adding pressure to injection site with gauze.

Single housed, 8-9 week old, male C57BL/6 mice were used for the in vivo study. FPLC purified modified synthetic leptin mRNA (SEQ ID NO:4) in which the uridines were substituted with pseudouridine was packaged in Cationic Lipid A (N:P molar ratio=8:1) and then were diluted in injectable saline at a dose of 10 μg per average group body weight.

On day 0, animals were weighed and sorted according to average body weight. Mice were dosed, and food intake (FI) was recorded, on each of days 1-7 and days 9, 11, and 16.

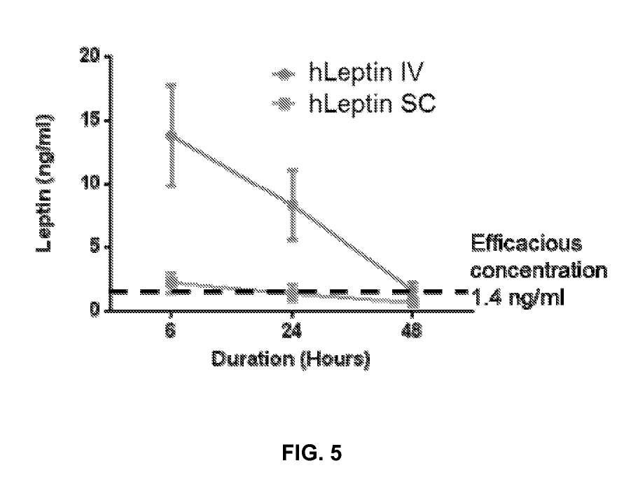

Human leptin protein levels were measured according to the ELISA protocol in EXAMPLE 1. Leptin protein levels after intravenous administration of leptin mRNA in Cationic Lipid A are detailed in TABLE 17 and FIG. 5.

TABLE 17
Leptin Protein Levels in Lean C57BL/6 Mice Dosed
Intravenously with Leptin mRNA in Lipid A

| Time point (hours) | Leptin protein (pg/mL) |
|---|---|
| 6 | 19566 |
| 24 | 19241 |
| 48 | 4029 |
| 120 | BLQ |

BLQ = below level of quantification

Intravenous delivery of human leptin mRNA to lean mice according to the procedure of this EXAMPLE led to 19 ng/mL leptin levels in plasma, which is over 10-fold above the 1.4 ng/mL EC₅₀ for suppression of food intake in leptin-deficient ob/ob mice, as determined in EXAMPLE 1.

### Example 8

### Mouse Subcutaneous Injection of Modified Synthetic Leptin mRNA

Prior to subcutaneous injection, mouse body weights were recorded and diet weighted, with mice grouped according to their body weights. The mice were manually restrained and placed on a work surface. Their scruffs were pinched and lifted away from the underlying muscle, the space into which was inserted a 25 gauge needle connected with a 1 mL syringe. The syringe plunger was pulled backwards in such a way as to ensure no fluid was drawn into the syringe, and then the desired volume of leptin mRNA was hand injected with moderate pressure and speed. The needle was then withdrawn and the mice returned to their cages.

8-9 week old, male C57BL/6 mice were used for the in vivo study. FPLC purified modified synthetic leptin mRNA (SEQ ID NO: 4) in which the uridines were substituted with pseudouridine (N:P molar ratio=8:1) packaged in Cationic Lipid A was diluted in injectable saline at a dose of 10 μg per average group body weight.

On day 0, animals were weighed and sorted according to average body weight. Mice were dosed at 9 AM and blood was taken at 9 AM on day 0. Blood was also taken at 9 AM on each of days 1 and 2 and assessed for leptin protein levels. Body weight and food intake were also recorded.

Human leptin protein levels were measured according to the ELISA protocol in EXAMPLE 1. Leptin protein levels after subcutaneous administration of leptin mRNA in Cationic Lipid A are detailed in TABLE 18 and FIG. 5.

TABLE 18
Leptin Protein Levels in Lean C57BL/6 Mice Dosed Subcutaneously
with Leptin mRNA in Cationic Lipid A

| Time point (hours) | Leptin protein (pg/mL) |
|---|---|
| 6 | 1238 |
| 24 | 1396 |
| 48 | 687 |
| 120 | BLQ |

BLQ = below level of quantification

Subcutaneous delivery of human leptin mRNA to lean mice according to the procedure of this EXAMPLE led to 1.4 ng/mL leptin protein levels in plasma, equivalent to the EC₅₀ for suppression of food intake in leptin-deficient ob/ob mice, as determined in EXAMPLE 1.

### Example 9

### Pharmacodynamics of Leptin mRNA Packaged with Cationic Lipid a and Administered to Leptin-Deficient Ob/Ob Mice

The purpose of this EXAMPLE was to demonstrate the specific effect human leptin on body weight and food intake of a mouse model of genetic leptin deficiency. The human leptin was delivered by administration of FPLC purified human leptin mRNA (SEQ ID NO: 4) in which the uridines were substituted with pseudouridine and the leptin mRNA was packaged in Cationic Lipid A and administered intravenously.

The mice used were 9 week old male ob/ob mice from Jackson Labs. Animals were single housed with a normal light cycle (6:00-18:00). They were given the modified synthetic leptin mRNA (SEQ ID NO: 4), or a control modified synthetic mouse erythropoietin mRNA (SEQ ID NO: 12), that had been HPLC purified, packaged in Cationic Lipid A (N:P molar ratio=4:1) and diluted in injectable phosphate buffered saline (PBS). Mice were given tail injections as described in EXAMPLE 7.

Three mice received PBS (Group A). Three mice received the modified synthetic leptin mRNA at a dose of 0.2 mpk (Group B). Three mice received the modified synthetic mouse erythropoietin mRNA at a dose of 0.2 mpk (Group C).

On day 0, mice were weighed and sorted into groups according to their body weights, so that the average body weight per group was the same, and then dosed at 9:00 AM based on their grouping. Their food was also weighed at the beginning of the study. All mice were weighed and their food was weighed at 9:00 AM on days 1, 2, 3, 4, 5, 6, 7 and 8.

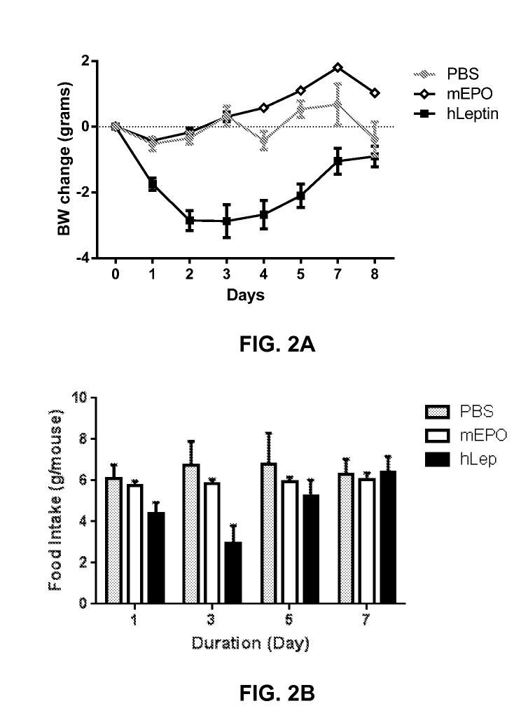

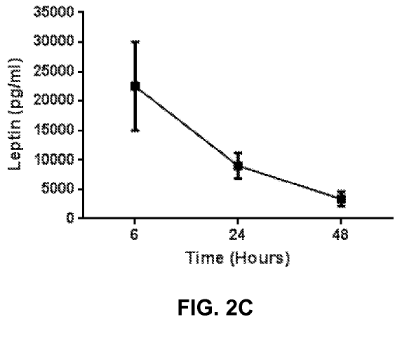

FIG. 2A and FIG. 2B B represent the body weight and food intake results of this study.

### Example 10

### Pharmacokinetics of Leptin mRNA Packaged with Cationic Lipid a and Administered to Leptin-Deficient Ob/Ob Mice

The purpose of this EXAMPLE was to demonstrate the expression levels of human leptin protein in a mouse model of genetic leptin deficiency after administration of FPLC purified human leptin mRNA (SEQ ID NO: 4) in which the uridines were substituted with pseudouridine, where the mRNA was packaged in Cationic Lipid A and administered intravenously.

The mice used were 9 week old male ob/ob mice from Jackson Labs. Animals were single housed with a normal light cycle (6:00-18:00). They were given the modified synthetic leptin mRNA (SEQ ID NO: 4) or a control modified synthetic mouse erythropoietin mRNA (SEQ ID NO: 12), that had been HPLC purified, packaged in Cationic Lipid A (N:P molar ratio=4:1), and diluted in injectable phosphate buffered saline (PBS). Mice were given tail injections as described in EXAMPLE 7.

Three mice received PBS (Group A). Three mice received the modified synthetic leptin mRNA at a dose of 0.2 mpk (Group B). Three mice received the modified synthetic mouse erythropoietin mRNA at a dose of 0.2 mpk (Group C).

On day 0, mice were weighed and sorted into groups according to their body weights, so that the average body weight per group was the same, and then dosed at 9:00 AM based on their grouping. All mice were also weighed at 9:00 AM on every subsequent day of the study.

Mice from each group were bled by tail nick on day 0 at 3:00 PM (6 hours post-dose), and then at 9:00 AM on days 1, 2, and 4. Plasma was isolated and human leptin or mouse erythropoietin levels are measured.

FIG. 2C represents the plasma human leptin level results of this study, as measured according to the protocol in EXAMPLE 1. For the work supporting FIG. 2C, mEPO protein expression was high in mice receiving mEPO mRNA (54,582 μg/mL at 6 hours), confirming mEPO mRNA delivery.

### Example 11

### Pharmacodynamics of Leptin mRNA Packaged with Cationic Lipid B and Administered to Leptin-Deficient Ob/Ob Mice

The purpose of this EXAMPLE was to demonstrate the effect human leptin on body weight and food intake of a mouse model of genetic leptin deficiency. The human leptin was delivered by administration of HPLC purified human leptin mRNA (SEQ ID NO: 4) in which the uridines were substituted with pseudouridine, the leptin mRNA was packaged in Cationic Lipid B and administered intravenously.

The animals used were 12 week old male ob/ob mice from Jackson Labs. Animals were single housed with a normal light cycle (6:00-18:00). They were given modified synthetic leptin mRNA (SEQ ID NO: 4) that had been HPLC purified, packaged in Cationic Lipid B (N:P molar ratio=4:1), and diluted in injectable phosphate buffered saline (PBS). Mice were given tail injections as described in EXAMPLE 7.

Five mice received PBS (Group A). Five mice received the modified synthetic leptin mRNA at a dose of 0.02 mpk (Group B). Five mice received the modified synthetic leptin mRNA at a dose of 0.06 mpk (Group C). Five mice received the modified synthetic leptin mRNA at a dose of 0.2 mpk (Group D).

On day 0, mice were weighed and sorted into groups according to their body weights, so that the average body weight per group was the same, and then dosed at 9:00 AM based on their grouping. Their food was also weighed at the beginning of the study. All mice were weighed and their food was weighed at 9:00 AM on days 1, 2, 3, 4, 5, 6, and 7.

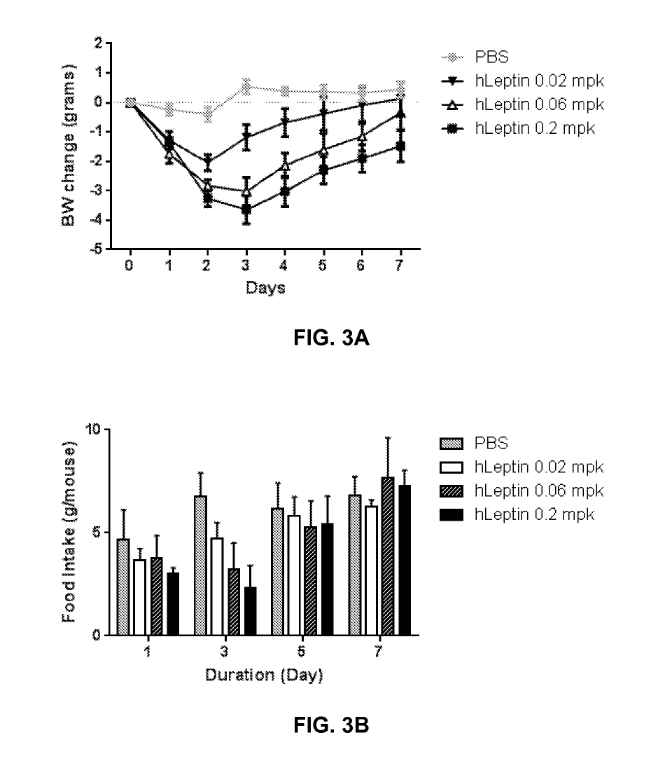

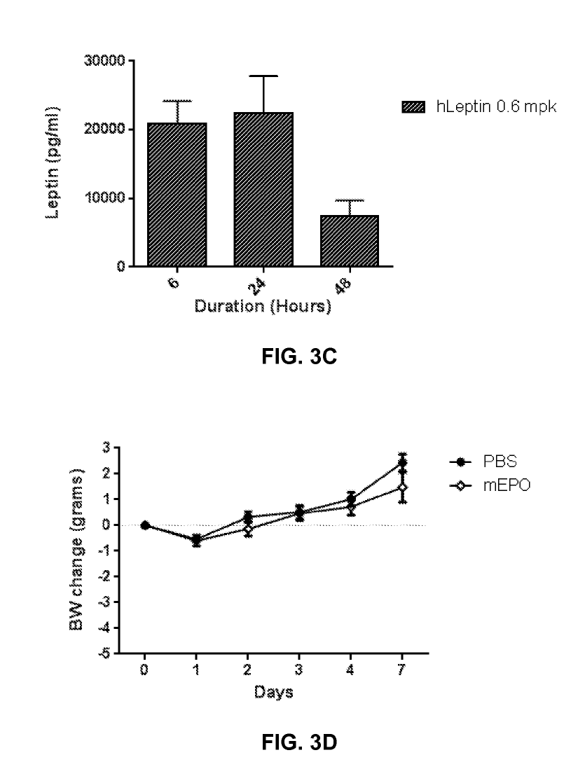

FIG. 3A and FIG. 3B represent the body weight and food intake results of this study.

### Example 12

### Pharmacokinetics of Leptin mRNA Packaged with Cationic Lipid B and Administered to Leptin-Deficient Ob/Ob Mice

The purpose of this EXAMPLE was to demonstrate the expression levels of human leptin protein in a mouse model of genetic leptin deficiency after administration of HPLC purified human leptin mRNA (SEQ ID NO:4) in which the uridines were substituted with pseudouridine, where the leptin mRNA was packaged in Cationic Lipid B and administered intravenously.

The animals used were 16 week old male ob/ob mice from Jackson Labs. Animals were single housed with a normal light cycle (6:00-18:00). They were given modified synthetic leptin mRNA (SEQ ID NO: 4) that had been HPLC purified, packaged in Cationic Lipid B (N:P molar ratio=4:1), and diluted in injectable phosphate buffered saline (PBS). Mice were given tail injections as described in EXAMPLE 7.

Five mice received PBS (Group A). Three mice received the modified synthetic leptin mRNA at a dose of 0.6 mpk (Group B).

On day 0, mice were weighed and sorted into groups according to their body weights, so that the average body weight per group was the same, and then dosed at 9:00 AM based on their grouping. All mice were also weighed at 9:00 AM on every subsequent day of the study.

Mice from each group were bled by tail nick on day 0 at 3:00 PM (6 hours post-dose), and then at 9:00 AM on days 1, 2, 3, 4, 5, 6, and 7. Plasma was isolated and human leptin protein levels were measured according to the ELISA protocol of EXAMPLE 1.

FIG. 3C represents the plasma human leptin protein results of this study.

### Example 13

### Pharmacodynamics of Leptin mRNA Packaged with Cationic Lipid C and Administered to Leptin-Deficient Ob/Ob Mice

The purpose of this EXAMPLE was to demonstrate the effect leptin protein on body weight and food intake of a mouse model of genetic leptin deficiency. The human leptin was delivered by administration of HPLC purified human leptin mRNA (SEQ ID NO: 4) in which the uridines were substituted with pseudouridine, packaged in Cationic Lipid C and administered intravenously.

The animals used were 10 week old male ob/ob mice from Jackson Labs. Animals were single housed with a normal light cycle (6:00-18:00). They were given modified synthetic leptin mRNA (SEQ ID NO: 4) that had been HPLC purified, packaged in Cationic Lipid C (N:P molar ratio=4:1) and diluted in injectable phosphate buffered saline (PBS). Mice were given tail injections as described in EXAMPLE 7.

Five mice received PBS (Group A). Five mice received the modified synthetic leptin mRNA at a dose of 0.02 mpk (Group B). Five mice received the modified synthetic leptin mRNA at a dose of 0.06 mpk (Group C). Five animals received the modified synthetic leptin mRNA at a dose of 0.2 mpk (Group D).

On day 0, mice were weighed and sorted into groups according to their body weights, so that the average body weight per group was the same, and then dosed at 9:00 AM based on their grouping. Their food was also weighed at the beginning of the study. All mice were weighed and their food was weight at 9:00 AM on days 1, 2, 3, 4, 5, 6, and 7.

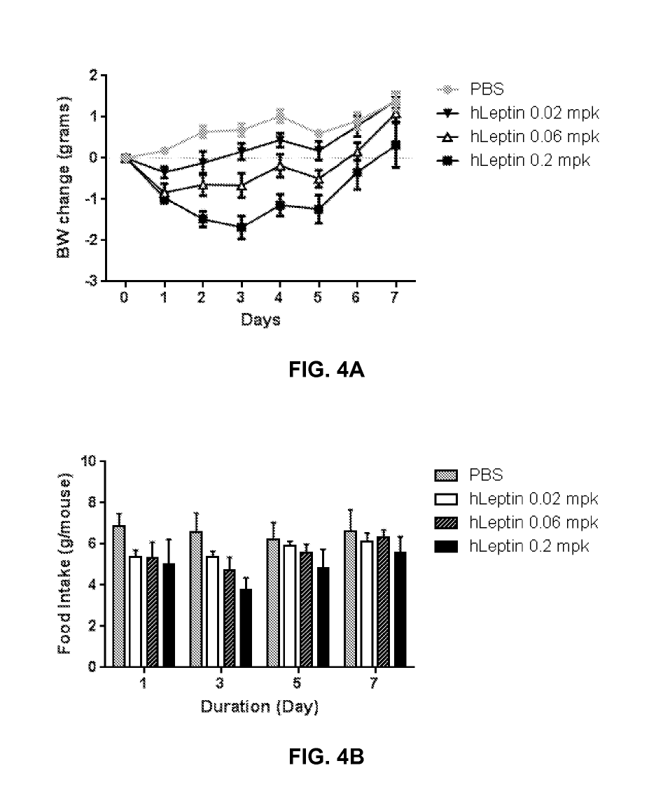

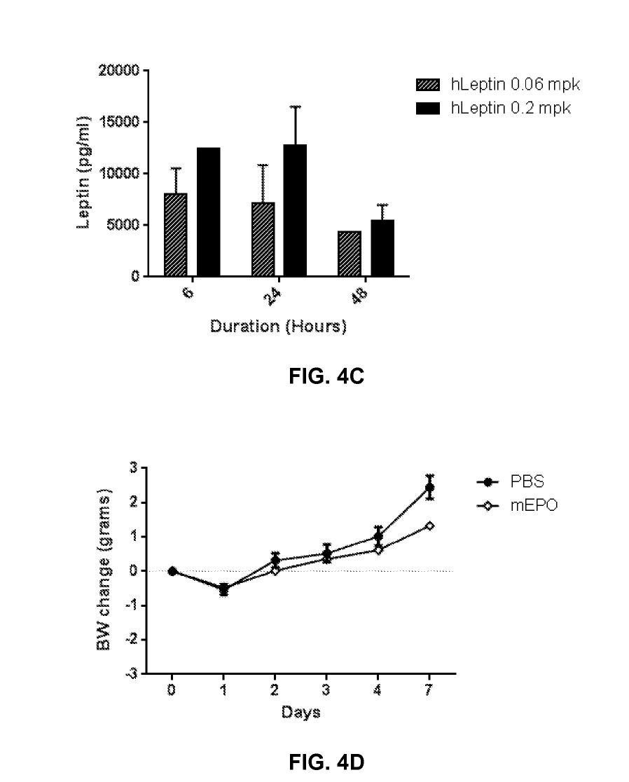

FIG. 4A and FIG. 4B represent the body weight and food intake results of this study.

### Example 14

### Pharmacokinetics of Leptin mRNA Packaged with Cationic Lipid C and Administered to Leptin-Deficient Ob/Ob Mice

The purpose of this EXAMPLE was to demonstrate the expression levels of human leptin protein after HPLC purified human leptin mRNA (SEQ ID NO: 4), in which the uridines were substituted with pseudouridine, was packaged in Cationic Lipid C and administered intravenously to a mouse model of genetic leptin deficiency.

The animals used were 10 week old male ob/ob mice from Jackson Labs. Animals were single housed with a normal light cycle (6:00-18:00). They were given the modified synthetic leptin mRNA of the invention (SEQ ID NO: 4) that had been HPLC purified, packaged in Cationic Lipid C at an N:P molar ratio of 4:1, and diluted in injectable phosphate buffered saline (PBS). Mice were given tail injections as described in EXAMPLE 7.

Three mice received PBS (Group A). Three mice received the modified synthetic leptin mRNA at a dose of 0.02 mpk (Group B). Three mice received the modified synthetic leptin mRNA at a dose of 0.06 mpk (Group C). Three mice received the modified synthetic leptin mRNA at a dose of 0.2 mpk (Group D).

On day 0, mice were weighed and sorted into groups according to their body weights, so that the average body weight per group was the same, and then dosed at 9:00 AM based on their grouping. All mice were also weighed at 9:00 AM on every subsequent day of the study.

Mice from each group were bled by tail nick on day 0 at 3:00 PM (6 hours post-dose), and then at 9:00 AM on days 1, 2, 3, 4, 5, 6, and 7. Plasma was isolated and human leptin protein levels were measured according to the ELISA protocol in EXAMPLE 1.

FIG. 4C represents the plasma human leptin protein results of this study.

### Example 15

### Pharmacokinetics of Mouse Erythropoietin mRNA Packaged with Cationic Lipid B or Cationic Lipid C and Administered to Leptin-Deficient Ob/Ob Mice

The purpose of this EXAMPLE was to demonstrate the lack of an effect on body weight and food intake of a mouse model of genetic leptin deficiency after administration of HPLC purified mouse erythropoietin mRNA (SEQ ID NO: 12), in which the uridines were substituted with pseudouridine, packaged in Cationic Lipid B or Cationic Lipid C and administered intravenously.

The animals used were 8 week old male ob/ob mice from Jackson Labs. Animals were single housed with a normal light cycle (6:00-18:00). They were given modified synthetic mouse erythropoietin (mEPO) mRNA (SEQ ID NO: 12) that had been HPLC purified, packaged in Cationic Lipid B or Cationic Lipid C at an N:P molar ratio of 4:1, and diluted in injectable phosphate buffered saline (PBS). Mice were given tail injections as described in EXAMPLE 7.

Five mice received PBS (Group A). Five mice received the modified synthetic mEPO mRNA at a dose of 0.02 mpk in Cationic Lipid B (Group B). Five mice received the modified synthetic mEPO mRNA at a dose of 0.2 mpk in Cationic Lipid C (Group C).

On day 0, mice were weighed and sorted into groups according to their body weights, so that the average body weight per group is the same, and then dosed at 9:00 AM based on their grouping. Their food was also weighed at the beginning of the study. All mice were weighed and their food was weighed at 9:00 AM on days 1, 2, 3, 4, and 7.

FIG. 3D and FIG. 4D represent the results of this study. For the work supporting FIG. 3D, mEPO protein expression was high in mice receiving mEPO mRNA (892,633 μg/mL at 6 hours), confirming mEPO mRNA delivery. For the work supporting FIG. 4D, mEPO protein expression was high in mice that received mEPO mRNA (158,865 μg/mL at 6 hours), confirming mEPO mRNA delivery.

### Example 16

### Toxicity and Tolerability Study of Modified Synthetic Leptin mRNA Packaged with Cationic Lipid C in C57BL/6 Mice

This EXAMPLE describes how HPLC purified modified synthetic leptin mRNA (SEQ ID NO: 4) packaged with Cationic Lipid C and administered intravenously (4 mpk) and subcutaneously (2 mpk) were generally safe in mice.

Male C57BL/6 mice (3 animals/group) received either phosphate buffered saline (PBS) subcutaneously or intravenously and served as controls or were dosed with the modified synthetic leptin mRNA (SEQ ID NO: 4) formulated in a lipid nanoparticle with Cationic Lipid C as the cationic lipid. TABLE 19 outlines the study design. TABLE 20 outlines the formulation characteristics. The mice in the intravenous arm of the study received approximately 10 mL/kg dose volumes. Mice dosed subcutaneously were shaved prior to dosing. At the termination of the study (approximately 24 and 72 hours after dosing), all mice assigned to the appropriate segment of the study were submitted for necropsy, their final body weights were recorded and blood samples were collected for clinical chemistry.

TABLE 19
Study Design and Animal Allocation

| | | | Route of | Termination |
|---|---|---|---|---|
| | | Dose | adminis- | (hours |
| Group | mRNA | (mg/kg) | tration | post-dose) |
| A^{a} | PBS | 0 | sc | 24 |
| B^{a} | modified synthetic leptin | 2 | sc | 24 |
| | mRNA (SEQ ID NO: 4 | | | |
| | packaged with Cationic | | | |
| | Lipid C | | | |
| C^{a} | PBS | 0 | sc | 72 |
| D^{a} | modified synthetic leptin | 2 | sc | 72 |
| | mRNA (SEQ ID NO: 4) | | | |
| | packaged with Cationic | | | |
| | Lipid C | | | |
| A^{b} | PBS | 0 | IV | 72 |
| B^{b} | modified synthetic leptin | 4 | IV | 72 |
| | mRNA (SEQ ID NO: 4) | | | |
| | packaged with Cationic | | | |
| | Lipid C | | | |

^{a}= subcutaneous study, see EXAMPLE 8
^{b}= intravenous study, see EXAMPLE 7

TABLE 20
Formulation Characteristics

| Formu- | Diameter | | | | | | |
|---|---|---|---|---|---|---|---|
| lation | (nm) | PDI^{a} | Encap^{b} | μg/mL | ml | μg | Yield |
| modified | 160.6 | 0.09 | 88.3 | 222.5 | 2 | 445.0 | 12.7 |
| synthetic | | | | | | | |
| leptin | | | | | | | |
| mRNA | | | | | | | |
| (SEQ ID | | | | | | | |
| NO: 4) | | | | | | | |
| packaged | | | | | | | |
| with | | | | | | | |
| Cationic | | | | | | | |
| Lipid C | | | | | | | |
| modified | 168.2 | 0.121 | 88.3 | 512.9 | 2.5 | 1282.3 | 36.6 |
| synthetic | | | | | | | |
| leptin | | | | | | | |
| mRNA | | | | | | | |
| (SEQ ID | | | | | | | |
| NO: 4) | | | | | | | |
| packaged | | | | | | | |
| with | | | | | | | |
| Cationic | | | | | | | |
| Lipid C | | | | | | | |

^{a} = polydispersity index;
^{b} = % of encapsulation of mRNA as determined by a Ribogreen assay in the presence of Triton-X;
^{c} = wash for subcutaneous dosing;
^{d} = IV dosing

Blood samples were collected by cardiac puncture and kept in plain tubes. Using a Cobas® 6000 analyzer (Roche Diagnostics), the following parameters were determined: Aspartate Transaminase (AST); Total Bilirubin; Alanine Transaminase (ALT); Total Protein; Alkaline Phosphatase; Albumin; Urea; Globulin; Creatinine; Creatine Kinase (CK); Glucose; Albumin/globulin ratio.

Clinical chemistry changes occurred at 72 hours post-dose and are outlined in TABLE 21.

TABLE 21
Salient Clinical Chemistry Changes Associated with the
Administration of Modified Synthetic Leptin mRNA (SEQ
ID NO: 4) Packaged with Cationic Lipid C to Male Mice
Compared to Concurrent Controls 72 Hours Post-dose

| | Group 4 | Group 6 |
|---|---|---|
| Analyte | 2 mg/kg sc | 4 mg/kg IV |
| Total protein | −1.7% | −8.1% |
| Albumin | −3.4% | −6.4% |
| Globulin | +1.6% | −12.2% |
| Albumin/globulin ratio | −5.1% | +6.6% |
| BUN | — | −14.9% |

Mice dosed with the modified synthetic leptin mRNA (SEQ ID NO: 4) packaged with Cationic Lipid C intravenously had decreased protein values without corresponding microscopic changes. Additionally, decreased BUN in mice dosed intravenously suggested a decreased food intake.

The changes reflected minimal inflammation with subcutaneous administration of the modified synthetic leptin mRNA (SEQ ID NO: 4) packaged with Cationic Lipid C (decreased total protein with decreased albumin, increased globulin and decreased albumin globulin ratio). These changes corresponded to microscopic neutrophilic splenic infiltrates and serocellular crust of skin.

At necropsy, spleen, liver and kidney weights were recorded and these tissues along with lungs were fixed from all animals in 10% neutral buffered formalin and routinely processed to produce stained tissue sections suitable for microscopic evaluation.

There were no modified synthetic leptin mRNA-related organ weight changes detected. There were no modified synthetic leptin mRNA-related gross changes observed. Of the mice dosed subcutaneously with the modified synthetic leptin mRNA (SEQ ID NO: 4) packaged with Cationic Lipid C, test article-related skin changes were clearly present in only one animal at 72 hours post-dose (subcutaneous and dermal inflammatory cell infiltrates). Microscopic observations in the spleen (increased cellularity of the red pulp or neutrophilic infiltrates or both) were considered as reactive and secondary to any skin irritation.

Mice dosed with the modified synthetic leptin mRNA (SEQ ID NO: 4) packaged with Cationic Lipid C intravenously had abnormal intravascular cells in the liver, increased cellularity in the splenic red pulp, and pulmonary vasculopathy and thrombosis (1/3 animals).

One mouse dosed with the modified synthetic leptin mRNA (SEQ ID NO: 4) packaged with Cationic Lipid C intravenously had pulmonary vasculopathy at 72 hours post-dose.

### Example 17

### Modified Synthetic Leptin mRNA Packaged with Cationic Lipid C

### In Vivo Preliminary Preclinical Safety Study

The purpose of this EXAMPLE was to determine the protein expression from the administration of HPLC purified modified synthetic leptin mRNA (SEQ ID NO: 4), in which the uredines were substituted with pseudouridine, packaged in Cationic Lipid C delivered by intravenous (IV) injection the formulation to mice. Delivery of modified synthetic leptin mRNA induced expression of leptin protein, as measured by an ELISA assay. Toxicity and tolerability was also analyzed.

Mice were given tail injections as described in EXAMPLE 7, above.

The in vivo study was performed with 8-9 week old male ob/ob mice, housed singly. They were given HPLC purified modified synthetic leptin mRNA (SEQ ID NO: 4) (N:P molar ration=4:1) packaged in Cationic Lipid C, and diluted in injectable saline at doses of 4 mpk for this EXAMPLE. A saline control was used.

Three mice received saline control. One group of three mice each receive the modified synthetic leptin mRNA (SEQ ID NO: 4) packaged in Cationic Lipid C at a dose of 4 mpk. On day 0, mice were weighed and sorted into groups according to their body weights, and then dosed based on their grouping. Food weight was recorded.

On day 1 (24 hours post-dose), all mice were sacrificed and records are taken of body weight and food weight, with visual observation of mouse behavior and activity

Blood by cardiac puncture is collected: serum for ALT/AST/ALP/Bilirubin (total and active) (150 to 250 μl); plasma for ELISA (15 μl).

Liver, spleen, and kidney were harvested. Whole tissue weights were recorded.

A dose proportional exposure of leptin protein was observed in the study. Modified synthetic leptin mRNA (SEQ ID NO: 4) packaged in Cationic Lipid C was dosed in lean C57BL/6 mice at a dose of 4 mpk, which is 20-fold above 0.2 mpk dose in ob/ob mice. A leptin protein level of 185 ng/mL in circulation was observed.

### Example 18

### The Modified Synthetic Leptin mRNA of the Invention Packaged with Cationic Lipid C

### In Vivo Pharmacokinetic and Efficacy Study with Repeated Doses

To determine the protein expression and in vivo efficacy of modified synthetic leptin mRNA packaged in Cationic Lipid C, repeatedly intravenous (IV) injections according to the protocol of EXAMPLE 7 were used to deliver modified synthetic leptin mRNA (SEQ ID NO: 4) packaged with Cationic Lipid C to mice. Delivery of modified synthetic leptin mRNA induced expression of leptin protein was followed by in vivo efficacy in ob/ob mice.

Mice were given tail injections as described in EXAMPLE 7.

The in vivo study was performed with 12 week old male ob/ob mice, housed singly. The mice were given HPLC purified modified synthetic leptin mRNA (SEQ ID NO: 4) packaged with Cationic Lipid C, diluted in injectable saline at doses of 0.2 mpk for the study. A saline control was used.

Pharmacokinetics.

One group of five mice received saline control, and one group of five mice received the HPLC purified human leptin mRNA of the invention (SEQ ID NO: 4), in which the uridines were substituted with pseudouridine, packaged with Cationic Lipid C at a dose of 0.2 mpk (10 μg). Separate groups of mice for measuring the kinetics of leptin protein expression and measuring the efficacy of leptin mRNA is necessary because the stress of the act of repeated blood sampling itself will cause the mice to lose weight. The mice were weighed and sorted on each of five days, so that the average body weight per group was the same. Food was weighed as well.

On day 0, the mice were dosed at 9 AM (in the morning) and were bled by tail nick at 3 PM (in the afternoon) for leptin analysis (15 μl of plasma). On days 3, 7, 10, 14, 14 and 17, the mice were dosed at 9 AM. On days 1, 2, 4, 8, 11, 15, and 18, the mice were weighed at 9 AM and bled by tail nick for leptin analysis (15 μl of plasma). Human leptin protein levels in the plasma are measured according to the ELISA protocol in EXAMPLE 1. Leptin protein levels measured in this EXAMPLE are in TABLE 22. Leptin protein levels following the first dose were assayed at time points 6, 24 and 48 hours, and subsequent protein levels were 24 hours following last dose.

TABLE 22
Leptin Protein Levels From Leptin mRNA in Cationic
Lipid C, Multi-dose Study in ob/ob Mice

| Time point (days) | Leptin protein (pg/mL) |
|---|---|
| 0.25 | 7776 |
| 1 | 3800 |
| 2 | 639 |
| 4 | 11082 |
| 11 | 7339 |
| 15 | 2625 |
| 18 | 2479 |

Pharmacodynamics.

Six animals received saline control. One group of six mice apiece received HPLC purified modified synthetic leptin mRNA (SEQ ID NO: 4), in which the uredines were substituted with pseudouridine, packaged with Cationic Lipid C at a dose of 0.2 mpk (10 μg). The animals were weighed and sorted, so that the average body weight per group was the same.

On day 1, the mice were dosed at 9 AM (in the morning). On days 1, 2, 4, 8, 9, 11-13, 15, 16, 18-20, 22, and 23, only body weights and food intake were recorded. On days 3, 7, 10, 14, 17, and 21, body weight and food intakes were recorded first thing in the morning and the mice were dosed at 9 AM.

Repeated treatment of the modified synthetic leptin mRNA of the invention packaged with Cationic Lipid C yielded sustained body weight loss and leptin protein expression. The modified synthetic leptin mRNA (SEQ ID NO:4) packaged with Cationic Lipid C was dosed twice per week for three weeks (total of six doses represented by arrows) and following each dose, a decrease in body weight and maintenance of leptin protein levels were observed, as detailed in TABLE 23.

TABLE 23
Body Weights in Cationic Lipid C Multi-dose Study in ob/ob Mice

| | PBS | Leptin mRNA |
|---|---|---|
| Time point | Change in body weight | Change in body weight |
| (days) | (grams) | (grams) |
| 1 | 0.1 | −1.1 |
| 2 | 0.8 | −1.5 |
| 3 | 1.2 | −1.0 |
| 4 | 1.0 | −2.2 |
| 5 | 2.6 | −2.1 |
| 6 | 3.1 | −1.6 |
| 7 | 3.2 | −0.9 |
| 8 | 3.3 | −2.0 |
| 9 | 3.7 | −1.8 |
| 10 | 4.0 | −0.9 |
| 11 | 3.9 | −1.5 |
| 12 | 5.4 | −1.1 |
| 13 | 6.1 | −0.3 |
| 14 | 6.2 | 0.4 |
| 15 | 6.4 | −0.7 |
| 16 | 7.2 | 0.1 |
| 17 | 7.2 | 0.6 |
| 18 | 7.8 | 0.1 |
| 19 | 8.7 | 0.4 |
| 20 | 9.6 | 1.4 |

### Example 19

### Modified Synthetic Leptin mRNA Packaged with Cationic Lipid C; In Vivo Stability Study

To determine the stability of HPLC purified human leptin mRNA (SEQ ID NO: 4), in which the uredines were substituted with pseudouridine, packaged in Cationic Lipid C (N:P molar ratio=4:1), intravenous (IV) injections were used to deliver modified synthetic leptin mRNA packaged with Cationic Lipid C to mice at various time points. Delivery of leptin mRNA induced expression of leptin protein and expression levels were compared at all time points.

Mice were given tail injections as described in EXAMPLE 7.

The in vivo study was performed with 10 week old male C57BL/6 mice, housed singly. The mice are given HPLC purified modified synthetic leptin mRNA (SEQ ID NO: 4) packaged (N:P molar ration=4:1) in Cationic Lipid C, and diluted in injectable saline at doses of 0.4 mpk for the study. A saline control was used.

Four mice received saline control. One group of four mice apiece received the modified synthetic leptin mRNA (SEQ ID NO: 4) packaged with Cationic Lipid C at a dose of 0.4 mpk (10 μg).

On day 1, and on weeks 1, 2, 4 and 8, the animals were weighed and sorted so that the average body weight per group is the same. The mice were dosed at 9 AM, and then weighed and bled at 24 hours for leptin levels (3 aliquots).

The stability of the modified synthetic leptin mRNA (SEQ ID NO: 4) packaged with Cationic Lipid C is determined by measuring leptin protein expression according to the ELISA protocol in EXAMPLE 1 following intravenous delivery. The modified synthetic leptin mRNA packaged with Cationic Lipid C was used to dose mice at day 1, 7, 14, 28 and 56 following initial formulation date and leptin protein level was measured 24 hours after each dose. Fifty-six days after the modified synthetic leptin mRNA packaged with Cationic Lipid C was prepared, the leptin protein level after administration decreased ˜60%.

### Example 20

### Pharmacokinetics and Pharmacodynamics of Leptin mRNA Packaged in Cationic Lipid C in the Cynomolgus Monkey

The purpose of this EXAMPLE was to examine the pharmacokinetics (PK) and pharmacodynamics (PD) of leptin following intravenous administration of HPLC purified human leptin mRNA (SEQ ID NO: 4), in which the uridines were substituted with pseudouridines, packed in Cationic Lipid C at a dose of 0.6 mg/kg in monkeys. Leptin protein level in plasma, blood chemistry, and lipid level in plasma or serum were all investigated.

TABLE 24 outlines the study design for this EXAMPLE. TABLE 25 outlines the formulation characteristics. Blood samples were collected as outlined in TABLE 26. Blood samples were collected at the saphenous or femoral vein into a syringe. Whole blood was analyzed for blood chemistry. Serum was collected for serum chemistry and plasma was collected for Leptin ELISA and lipid metabolism.

Three monkeys were dosed intravenously with modified synthetic leptin mRNA (SEQ ID NO: 4) packaged with Cationic Lipid C at a dose of 0.6 mpk. The peak leptin expression of 129 ng/mL was observed at 4 hours following drug product treatment. This confirmed translation of leptin expression from rodent to cynomolgus monkey.

Under the conditions of the EXAMPLE, the modified synthetic leptin mRNA of the invention packaged with Cationic Lipid C was administered intravenously at a dose of 0.6 mg/kg to male cynomolgus monkeys produced inflammatory changes based on clinical pathology parameters and increased pro-inflammatory cytokines and chemokines. Additional clinical pathology alterations suggested liver and muscle changes. There was also an increase in the anti-inflammatory cytokine, IL-1 RA. All of these changes were most prominent at early time points and were mostly normal by 24 hours (in case of cytokines) or 72 hours (clinical pathology).

As described below, a single intravenous dose of the modified synthetic leptin mRNA (SEQ ID NO: 4) packaged with Cationic Lipid C at 0.6 mg/kg to cynomolgus monkeys was well tolerated and produced inflammatory changes and mostly minimal changes suggestive of liver and muscle damage.

TABLE 24
Study Design and Animal Allocation

| | | | | Return to |
|---|---|---|---|---|
| | Dose | Route of | Animal | colony (hrs. |
| mRNA | (mg/kg) | administration | numbers | post-dose) |
| The modified | 0.6 | IV | 1, 2, 3 | 72 |
| synthetic | | | | |
| leptin mRNA | | | | |
| (SEQ ID NO: 4) | | | | |
| packaged with | | | | |
| Cationic Lipid C | | | | |

TABLE 25
Formulation Characteristics

| | Dia- | | | | | |
|---|---|---|---|---|---|---|
| | meter | | | | | |
| Formulation | (nm) | PDI^{a} | μg/mL | ml | μg | Yield |
| The modified | 147.5 | 0.14 | 308.2 | 2.5 | 1217 | 61 |
| synthetic leptin | | | | | | |
| mRNA (SEQ ID | | | | | | |
| NO: 4) packaged | | | | | | |
| with Cationic | | | | | | |
| Lipid C | | | | | | |

^{a} = polydispersity index

TABLE 26
Allocation of Blood Samples from Each Animal

| Time point | | Clinical | |
|---|---|---|---|
| (hour) | Hematology | chemistry | Immunotoxicity |
| 0 | x | x | x |
| 2 | | | x |
| 4 | | | x |
| 6 | | | x |
| 24 | x | x | x |
| 72 | x | x | x |

Blood samples for hematology were collected into EDTA anticoagulant. The following parameters were determined, using an ADVIA 2120® analyzer: Red Blood Cell Count, White Blood Cell Count, Hemoglobin, Neutrophil Count, Hematocrit, Lymphocyte Count, Mean Corpuscular Volume, Monocyte Count, Mean Corpuscular Hemoglobin, Eosinophil Count, Mean Corpuscular Hemoglobin Concentration, Basophil Count, Reticulocyte Count, Large Unstained Cells Count, and Platelet Count.

Blood samples were collected by cardiac puncture and kept in plain tubes. Using a Cobas 6000 analyzer, the following parameters were determined: Aspartate Transaminase (AST); Total Bilirubin; Alanine Transaminase (ALT); Total Protein; Alkaline Phosphatase; Albumin; Urea; Globulin; Creatinine; Creatine Kinase (CK); Glucose; Albumin/globulin ratio; Sodium; Phosphorous; Potassium; Cholesterol; Chloride; Triglycerides; Calcium; and Magnesium.

The Invitrogen Cytokine Monkey Magnetic 28-Plex Panel (Kit Catalog Number: LPC0003M, Invitrogen Corporation, Carlsbad Calif.) was used for the determination of analytes listed in TABLE 27. Values below the lower limit of quantitation were entered into the data tables as Lower Limit of Quantification (LLOQ), based on a dilution factor of 1:2. In some instances the LLOQ was different because a different dilution was used for its calculation. The Bioplex 200 (Bio-Rad Laboratories, Inc., Hercules, Calif.) was used for the analysis (Software version: Bio-Plex Manager™ 5.0 Security Edition, Build 531) and Microsoft Excel® 2010 was used to facilitate data handling and included in the raw data.

TABLE 27
Analytes Tested and Their Limits of Detection

| | LLOQ of assay | ULOQ of assay |
|---|---|---|
| Analyte | pg/mL | pg/mL |
| IL-1β | 13.91 | 1,780.00 |
| IL-1RA | 470.94 | 60,280.00 |
| IL-2 | 149.57 | 19,145.00 |
| IL-4 | 213.59 | 27,340.00 |
| IL-5 | 10.08 | 1,290.00 |
| IL-6 | 29.57 | 3,785.00 |
| IL-8 | 22.93 | 2,935.00 |
| IL-10 | 57.42 | 7,350.00 |
| IL-12 | 516.88 | 66,160.00 |
| IL-15 | 140.74 | 18,015.00 |
| IL-17 | 89.26 | 11,425.00 |
| EGF | 22.66 | 2,900.00 |
| Eotaxin | 24.53 | 3,140.00 |
| FGF | 14.22 | 1,820.00 |
| G-CSF | 423.83 | 54,250.00 |
| GM-CSF | 132.42 | 16,950.00 |
| HGF | 114.80 | 14,695.00 |
| IFN-γ | 44.18 | 5,655.00 |
| I-TAC | 149.73 | 19,165.00 |
| MCP-1 | 70.51 | 9,025.00 |
| MDC | 914.57 | 117,065.00 |
| MIF | 41.21 | 5,275.00 |
| MIG | 41.68 | 5,335.00 |
| MIP-1α | 103.40 | 13,235.00 |
| MIP-1β | 115.63 | 14,800.00 |
| RANTES | 55.86 | 7,150.00 |
| TNF-α | 73.32 | 9,385.00 |
| VEGF | 4.57 | 585.00 |

Results.

No significant hematological changes occurred. The clinical chemistry changes are outlined in TABLE 28. Intravenous administration of the modified synthetic leptin mRNA of the invention packaged with Cationic Lipid C at a dose of 0.6 mg/kg caused clinical chemistry alterations at both time points, reflecting minimal hepatocellular changes (increased AST, ALT, decreased albumin) and potential muscle changes at 24 hours post-dose (increased AST and CK). Subtle inflammatory changes were indicated by decreased albumin (in addition to increases in pro-inflammatory cytokines detailed below). Decreased triglycerides primarily at 24 hours post-dose may have reflected an impact on lipid metabolism, decreased food intake or could be due to biological variation.

TABLE 28
Salient Clinical Chemistry Changes Associated with the
Administration of the Modified Synthetic Leptin mRNA
of the Invention Packaged with Cationic Lipid C to Male
Cynomolgus Monkeys Compared to Pre-dose Values

| Analyte | 24 hrs post-dose | 72 hrs post-dose. |
|---|---|---|
| AST | +2.7-fold | +1.8-fold (#3 only) |
| ALT | +1.7-fold | +2.2-fold |
| CK | +1030% | +139% (#3 only) |
| Albumin | −9.6% | −8.4% |
| Triglycerides | −45.3% | −22% |

Increased cytokine, chemokine and growth factor concentrations were noted following dosing with the modified synthetic leptin mRNA of the invention packaged with Cationic Lipid C at a dose of 0.6 mg/kg iv when values were compared to pre-dose values. Fold changes were calculated using the LLOQ if the analyte pre-dose values were below Lower Limit of Quantitiation (LLOQ). The Upper Limit of Quantitation (ULOQ) was used to calculate fold increase when the serum analyte level was above the ULOQ (MCP-1 at 6 hours for animal #3).

Several of the test article-related changes were noted in all three animals as early as 2 hours post-dose and most of these changes returned to pre-dose levels by 24 hours post-dose. There were also a few analytes that were elevated only in one of the animals. The relationship of these latter changes to treatment cannot be ruled out because they were above pre-dose values and because other cytokines were increased in the same monkeys.

Increases occurred in all three monkeys in serum IL1RA levels from 2 hours post-dose through 6 hours post-dose inclusively, with levels returning to normal by 24 hours post-dose; the maximal increase was approximately 12-fold in magnitude in at least one animal at one time point. Concurrently, serum IL6 levels were elevated (over 100-fold in at least one monkey at one time point) from 2 hours post-dose through 6 hours post-dose in all animals returning to pre-dose values by 24 hours. Serum MCP-1 levels were increased at 2 hours post-dose including an increase up to over 100-fold at 6 hours post-dose with an approximately 2-fold increase at 24 hours post-dose and pre-dose values at 72 hours post-dose. Serum levels of I-TAC (CXCL11 chemokine) were increased in all three animals at 4 hours post-dose and remained elevated at 6 hours (by up to 78-fold in one animal). By 24 hours I-TAC levels were elevated by up to 8-fold and returned to pre-dose levels by 72 hours post-dose. Additionally, approximately 3-fold increases occurred in serum VEGF levels in animals #1 and #3, but not in animal #2. These increases were seen at 2, 4 or 6 hours post-dose. TABLE 29 outlines the changes over time for the four analytes with the most consistent elevations.

TABLE 29
Salient Changes in Serum Cytokine and Chemokine Levels Associated
with the Administration of the Modified Synthetic Leptin
mRNAof the Invention Packaged with Cationic Lipid C to Male
Cynomolgus Monkeys Compared to Pre-dose Values

| | 2 hr | 4 hr | 6 hr | 24 hr | 72 hr |
|---|---|---|---|---|---|
| Analyte | post-dose | post-dose | post-dose | post-dose | post-dose |
| Animal 1 | | | | | |
| IL1RA | —^{a} | 12.7x | 7.3x | — | — |
| IL6 | 16.4x | 52.1x | 20.2x | — | — |
| MCP-1 | 46x | 64.5x | 21.9x | 1.5x | — |
| I-TAC | — | 15.2x | 44.4x | 4.3x | — |
| Animal 2 | | | | | |
| IL1RA | 1.7x | 3.2x | 4.1x | — | — |
| IL6 | 22.4x | 8.1x | 9.9x | — | — |
| MCP-1 | 33.2x | 20x | 18.7x | 2x | 1.3x |
| I-TAC | — | 12.4x | 38.6x | 3.6x | — |
| Animal 3 | | | | | |
| IL1RA | 6.9x | 5.7x | 10.5x | — | — |
| IL6 | 111.2x | 21.9x | 86.1x | — | — |
| MCP-1 | 83.5x | 35x | 105.4x^{b} | 2.3x | 1.4x |
| I-TAC | — | 14x | 78.3x | 8.4x | — |

^{a}= unchanged/below baseline or value recorded as LLOQ;
^{b}= value above ULOQ

TABLE 29 shows that salient changes were observed in serum cytokine and chemokine levels associated with the administration of the modified synthetic leptin mRNA of the invention packaged with Cationic Lipid C to male cynomolgus monkeys compared to pre-dose values.

Under the conditions of the EXAMPLE, the modified synthetic leptin mRNA of the invention packaged with Cationic Lipid C construct administered intravenously at 0.6 mg/kg to male cynomolgus monkeys produced inflammatory changes based on clinical pathology parameters and increased pro-inflammatory cytokines and chemokines. Additional clinical pathology alterations suggested liver and muscle changes. There was also an increase in the anti-inflammatory cytokine, IL-1RA. All of these changes were most prominent at early time points and were mostly normal by 24 hours (in case of cytokines) or 72 hours (clinical pathology).

Leptin itself has an immunomodulatory effect through increasing both pro- and anti-inflammatory cytokines (stimulating TNFα, IL-6 and IFNγ as well as IL-1 RA in vitro in peripheral blood mononuclear cells. See, Juge-Aubry C E and Meier C A (2002) Mol. Cell Endocrinol. 194(1-2): 1-7).

In conclusion, a single intravenous dose of the modified synthetic leptin mRNA of the invention packaged with Cationic Lipid C to cynomolgus monkeys produced inflammatory changes that resolved after 72 hours and mostly minimal changes suggestive of liver and muscle damage. Based on the results of this EXAMPLE, the dose of 0.6 mg/kg was well tolerated in cynomolgus monkeys.

### Example 21

### The Modified Synthetic Leptin mRNA of the Invention Packaged with Cationic Lipid B

### In Vivo Preliminary Preclinical Safety Study

The in vivo study in this EXAMPLE was performed with 8-9 week old male C57BL/6 mice, housed singly. The mice were given HPLC purified modified synthetic leptin mRNA (SEQ ID NO: 4), in which the uridines were substituted with pseudouridine, packaged in Cationic Lipid B (N:P molar ratio=4:1), and diluted in injectable saline at doses of 7 mpk for the study. A saline control was used.

On day 0, mice were given intravenous tail injections as described in EXAMPLE 7.

As detailed in TABLE 30, three mice receive saline control (Group A) and one group of three mice each receive the modified synthetic leptin mRNA packaged with Cationic Lipid B at a dose of 7 mpk (Group B). Group A and B mice were sacrificed on day 1. Three mice receive saline control (Group C), and one group of three mice each receive the modified synthetic leptin mRNA packaged with Cationic Lipid B at a dose of 7 mpk (Group D). Group C and D mice were sacrificed on day 3. TABLE 31 outlines the formulation characteristics.

TABLE 30
Study Design and Animal Allocation

| | | Dose | Animal | Termination |
|---|---|---|---|---|
| Group | mRNA | (mg/kg) | numbers | (hrs. post-dose |
| A | PBS | 0 | A1-A3 | 24 |
| B | modified synthetic leptin | 7 | B1-B3 | 24 |
| | mRNA (SEQ ID NO: 4) | | | |
| | packaged with Cationic | | | |
| | Lipid B | | | |
| C | PBS | 0 | C1-C3 | 72 |
| D | modified synthetic leptin | 7 | D1-D3 | 72 |
| | mRNA (SEQ ID NO: 4) | | | |
| | packaged with Cationic | | | |
| | Lipid B | | | |

TABLE 31
Formulation Characteristics

| | Diameter | | | | | | |
|---|---|---|---|---|---|---|---|
| Formulation | (nm) | PDI^{a} | Encap^{b} | μg/mL | ml | μg | Yield |
| modified synthetic | 148.4 | 0.113 | 88.7 | 768 | 2.5 | 1921 | 48.0 |
| leptin mRNA | | | | | | | |
| (SEQ ID NO: 4) | | | | | | | |
| packaged with | | | | | | | |
| Cationic Lipid B | | | | | | | |

^{a} = polydispersity index
^{b} = % of encapsulation of mRNA as described in EXAMPLE 5

On day 0, mice were weighed and sorted into groups according to their body weights, and then dosed based on their grouping. Food weight was recorded.

On day 1, all mice from Groups A and B were sacrificed and records were taken of body weight and food weight, with visual observation of mouse behavior and activity. On day 3, all mice from Groups C and D were sacrificed and records were taken of body weight and food weight, with visual observation of mouse behavior and activity.

Blood by cardiac puncture was collected: serum for ALT/AST/ALP/Bilirubin (total and active) (150 to 250 μL); plasma for leptin protein measurement (15 μL) and Cationic Lipid measurement (20 μL).

Liver, spleen, and kidney are harvested: whole tissue weights were recorded

A dose proportional exposure of leptin protein in tolerability study was observed. Modified synthetic leptin mRNA of the invention (SEQ ID NO: 4) packaged in Cationic Lipid B was dosed in lean C57BL/6 mice at a dose of 7 mpk. A leptin protein level of 1300 ng/mL was observed in circulation as measured according to the ELISA assay protocol in EXAMPLE 1, which is 17-fold above what was observed for a 0.6 mpk dose in C57BL/6 mice.

Blood samples were collected by cardiac puncture and kept in plain tubes. Using a Cobas 6000 analyzer, the following parameters were determined: Aspartate Transaminase (AST); Total Bilirubin; Alanine Transaminase (ALT); Total Protein; Alkaline Phosphatase; Albumin; Urea; Globulin; Creatinine; Creatine Kinase (CK); Glucose; Albumin/globulin ratio.

At necropsy, spleen, liver and kidney weights were recorded and these tissues along with lungs were fixed from all animals in 10% neutral buffered formalin and routinely processed to tissue sections suitable for microscopic evaluation.

The clinical chemistry changes are outlined in TABLE 32.

TABLE 32
Salient Clinical Chemistry Changes Associated with the Administration
of the Modified Synthetic Leptin mRNA Packaged with Cationic
Lipid B to Male Mice Compared to Concurrent Controls

| | Group B | Group D |
|---|---|---|
| Analyte | 24 hrs. | 72 hrs. |
| Total protein | +5.0% | −4.7% |
| Albumin | −1.7% | −9% |
| Globulin | +20.9% | +5.8% |
| Albumin/globulin ratio | −19.7% | −14.9% |
| BUN | −22.6% | −19.6% |

The changes reflected minimal inflammation at both time points (decreased total protein with decreased albumin, increased globulin and decreased albumin globulin ratio). These changes microscopically corresponded to neutrophilic splenic infiltrates, to abnormal intravascular cells in hepatic vasculature or to pulmonary vasculopathy and thrombosis. Additionally, decreased BUN suggested decreased food intake.

Organ weights are presented in TABLE 33. The modified synthetic leptin mRNA-related increase occurred in spleen weights at approximately 72 hours post-dose was minimal (net 8% difference in the three mice) and corresponded to microscopic increased cellularity of the red pulp and basophilic cells.

TABLE 33
Mean Splenic Weight Changes at 72 Hours Post-dose

| | Mean terminal | | Spleen relative |
|---|---|---|---|
| Group | body weight (g) | Spleen (mg) | to body % |
| Control | 27.9 | 68.5 | 0.245 |
| (Group 3) | | | |
| Cationic | 25.47 | 82.1 | 0.324 |
| Lipid B | | | (net 8% difference) |
| (Group 4) | | | |

The modified synthetic leptin mRNA-related splenic and liver changes were present in dosed mice at 24 hours post-dose whereas at 72 hours post-dose in addition to changes in the liver and spleen, morphological changes were present in the lung as well. The modified synthetic leptin mRNA-related splenic changes at 24 hours post-dose included cellular debris/single cell necrosis in the white and red pulp, decreased cellularity of the red pulp, neutrophilic infiltrate, and basophilic cells. The splenic basophilic cells most likely corresponded to early megakaryocytes. These changes were in keeping with the inflammatory profile observed in clinical pathology.

In the liver, abnormal intravascular cells were present in animals dosed with the modified synthetic leptin mRNA of the invention packaged with Cationic Lipid B, further indicating an inflammatory response.

At 72 hours post-dose, abnormal intravascular cells were still present in the liver. There were also splenic changes characterized by cellular debris and increased cellularity of the red pulp or basophilic cells (all indicating extramedullary hematopoiesis).

In the lung at 72 hours, vasculopathy and thrombi were present in medium and small caliber vasculature.

Additionally, the red pulp was somewhat distended and rarefied in all animals (but more so in those treated with the modified synthetic leptin mRNA of the invention packaged with Cationic Lipid B) at both time points examined.

Under the conditions of the EXAMPLE, the modified synthetic leptin mRNA of the invention packaged with Cationic Lipid B produced spleen changes consistent with an inflammatory clinical pathology profile, cellular debris/single cell necrosis, neutrophilic infiltrate, and basophilic cells (early megakaryocytes) and abnormal intravascular cells in the liver at 24 hours. In addition to these changes it also produced pulmonary vasculopathy by 72 hours.

### Example 22

### The Modified Synthetic Leptin mRNA of the Invention Packaged with Cationic Lipid B

### In Vivo Pharmacokinetic and Efficacy Study with Repeated Doses

To determine the protein expression and in vivo efficacy of the modified synthetic leptin mRNA (SEQ ID NO: 4) packaged with Cationic Lipid B, repeated intravenous (IV) injections were used to deliver the modified synthetic leptin mRNA to mice. Delivery of leptin mRNA induced expression of leptin protein was followed by in vivo efficacy in ob/ob mice.

Mice were given tail injections as described in EXAMPLE 7, above.

The in vivo study of this EXAMPLE was performed with 16 week old male ob/ob mice, housed singly. The mice were given HPLC purified modified synthetic leptin mRNA (SEQ ID NO: 4) (N:P molar ratio=4:1) packaged in Cationic Lipid B, diluted in injectable saline at doses of 0.2 mpk for the study. A saline control was used.

Pharmacokinetics.

Five animals received saline control. One group of five mice each receive the modified synthetic leptin mRNA (SEQ ID NO: 4) packaged with Cationic Lipid B at a dose of 0.2 mpk (10 μg). The animals were weighed and sorted on each of five days, so that the average body weight per group is the same. Food will be weighed as well.

On day 0, the mice were dosed at 9 AM and were bled by tail nick at 3 PM for leptin (15 μl of plasma). On days 1, 2, 4, 8, 11, 15, and 18, the animals are weighed at 9 AM and bled by tail nick for leptin (15 μl of plasma). Body weight and food intake are recorded. On days 3, 7, 10, 14, and 17, the mice are dosed at 9 AM and body weight and food intake are recorded. On days 9 and 21, only body weight and food intake were recorded.

Leptin protein levels were measured following the first dose at time points 6, 24 and 48 hours. Leptin protein levels were measured 24 hours after subsequent doses. The results in TABLE 34 demonstrate that repeated treatment of the modified synthetic leptin mRNA of the invention packaged with Cationic Lipid B yield similar levels of leptin protein expression.

TABLE 34
Leptin Protein Levels in Cationic Lipid
B Multi-dose Study in ob/ob Mice

| Time point (days) | Leptin protein (pg/mL) |
|---|---|
| 0.25 | 76758 |
| 1 | 61500 |
| 2 | 12128 |
| 4 | 86757 |
| 8 | 17510 |

Pharmacodynamics.

Six animals received saline control. One group of six mice apiece received the modified synthetic leptin mRNA (SEQ ID NO: 4) packaged with Cationic Lipid B at a dose of 0.2 mpk (10 μg). The mice were weighed and sorted, so that the average body weight per group is the same.

On days 0, 3, and 7, the mice are dosed at 9 AM and body weight and food intake were recorded. On days 1, 2, 5, 6, and 8-21, only body weights and food intakes were recorded.

Repeated treatment of the modified synthetic leptin mRNA of the invention packaged with Cationic Lipid B yield sustained body weight loss and leptin protein expression. The ob/ob mice were treated with the modified synthetic leptin mRNA (SEQ ID NO: 4) packaged with Cationic Lipid B three times on days 1, 3 and 7. After each dose, mice continued to lose significant amount of body weight.

TABLE 35
Body weights in Cationic Lipid B Multi-dose study in ob/ob mice

| | PBS | Leptin mRNA |
|---|---|---|
| Time point | Change in body weight | Change in body weight |
| (days) | (grams) | (grams) |
| 1 | 0.0 | −2.7 |
| 2 | 0.5 | −4.6 |
| 3 | 0.9 | −5.3 |
| 4 | 0.8 | −7.0 |
| 5 | 1.4 | −8.3 |
| 6 | 1.6 | −9.1 |
| 7 | 2.1 | −8.7 |
| 8 | 1.4 | −10.0 |
| 9 | 2.2 | −10.7 |
| 10 | 1.6 | −10.7 |

Leptin protein expression remained at 25 ng/mL. Leptin protein level following the first dose at time points 6, 24 and 48 hours and subsequent protein levels are 24 hours following last dose.

### Example 23

### Pharmacokinetics and Pharmacodynamics of Leptin mRNA Packaged in Cationic Lipid B in the Cynomolgus Monkey

The objective of this EXAMPLE is to examine the pharmacokinetics and pharmacodynamics (PD) of leptin in monkeys following intravenous administration of 0.6 mg/kg HPLC purified human leptin mRNA (SEQ ID NO: 4) in which the uridines were substituted with pseudouridine, where the mRNA was packaged in Cationic Lipid B. Leptin protein level in plasma, blood chemistry, and lipid level in plasma or serum were all investigated.

Blood samples were collected at the saphenous or femoral vein into a syringe. TABLE 36 outlines the study design. TABLE 37 outlines the formulation characteristics. Blood samples were collected as outlined in TABLE 38. Whole blood, plasma and serum were collected based on time points listed in TABLE 38. Whole blood was analyzed for blood chemistry. Serum was collected for serum chemistry and plasma was collected for leptin ELISA and lipid metabolism. Three monkeys were dosed intravenously with the modified synthetic leptin mRNA packaged with Cationic Lipid B at 0.6 mpk. The peak leptin protein expression of 95 mg/mL was observed at 6 hours following drug product treatment, with leptin protein levels being measured by ELISA assay as described in EXAMPLE 1. This confirmed translation of leptin expression from rodent to cynomolgus monkey. Under the conditions of the EXAMPLE, the modified synthetic leptin mRNA of the invention (SEQ ID NO: 4) packaged with Cationic Lipid B administered intravenously at 0.6 mg/kg to male cynomolgus monkeys produced inflammatory changes based on clinical pathology parameters and increased pro-inflammatory cytokines and chemokines. Additional clinical pathology alterations suggested liver and muscle changes. There was also an increase in the anti-inflammatory cytokine, IL-1 RA. All of the cytokine changes were most prominent at 6 hours post-dose and were mostly normal by 24 hrs. Clinical pathology changes were prominent at 24 hours post-dose, but some changes persisted throughout the study.

As described below, a single intravenous dose of the modified synthetic leptin mRNA (SEQ ID NO: 4) packaged with Cationic Lipid B at 0.6 mg/kg to cynomolgus monkey was well tolerated.

TABLE 36
Study Design and Animal Allocation

| | | | | Return to |
|---|---|---|---|---|
| | Dose | Route of | Animal | colony (hrs. |
| mRNA | (mg/kg) | administration | numbers | post-dose) |
| modified syn- | 0.6 | IV | 1, 2, 3 | 72 |
| thetic leptin | | | | |
| mRNA (SEQ | | | | |
| ID NO: 4) | | | | |
| packaged with | | | | |
| Cationic Lipid B | | | | |

TABLE 37
Formula Characteristics

| | Diameter | | | | | | |
|---|---|---|---|---|---|---|---|
| Formulation | (nm) | PDI^{a} | Encap^{b} | μg/mL | ml | μg | Yield |
| modified | 118.3 | 0.072 | 92.8 | 141 | 16 | 2255 | 75.2 |
| synthetic leptin | | | | | | | |
| mRNA (SEQ ID | | | | | | | |
| NO: 4) | | | | | | | |
| packaged with | | | | | | | |
| Cationic Lipid B | | | | | | | |

^{a} = polydispersity index
^{b} = % of encapsulation of mRNA as determined by a Ribogreen assay in the presence of Triton-X

TABLE 38
Allocation of Blood Samples from Each Animal

| Time point | | | Clinical | |
|---|---|---|---|---|
| (hour) | Hematology | Coagulation | chemistry | Immunotoxicity |
| 0 | x | x | x | x |
| 1 | | | | x |
| 3 | | | | x |
| 6 | | | | x |
| 24 | x | x | x | x |
| 72 | x | | x | |

Blood samples for hematology were collected into EDTA anticoagulant. The following parameters were determined, using an ADVIA 2120® analyzer: Red Blood Cell Count, White Blood Cell Count, Hemoglobin, Neutrophil Count, Hematocrit, Lymphocyte Count, Mean Corpuscular Volume, Monocyte Count, Mean Corpuscular Hemoglobin, Eosinophil Count, Mean Corpuscular Hemoglobin Concentration, Basophil Count, Reticulocyte Count, Large Unstained Cells Count, and Platelet Count.

Blood samples for coagulation testing were collected into tubes containing trisodium citrate as coagulant. The following parameters were determined, using a Stago STA-R instrument: prothrombin time and activated partial thromboplastin time.

Blood samples were collected by cardiac puncture and kept in plain tubes. Using a Cobas 6000 analyzer, the following parameters were determined: Aspartate Transaminase (AST); Total Bilirubin; Alanine Transaminase (ALT); Total Protein; Alkaline Phosphatase; Albumin; Urea; Globulin; Creatinine; Creatine Kinase (CK); Glucose; Albumin/globulin ratio; Sodium; Phosphorous; Potassium; Cholesterol; Chloride; Triglycerides; Calcium; and Magnesium.

The Invitrogen Cytokine Monkey Magnetic 28-Plex Panel (Kit Catalog Number: LPC0003M, Invitrogen Corporation, Carlsbad Calif. USA) was used for the determination of analytes listed in TABLE 25 in EXAMPLE 20. The Bioplex 200 (Bio-Rad Laboratories, Inc., Hercules Calif. USA) was used for the analysis (Software version: Bio-Plex Manager™ 5.0 Security Edition, Build 531) and Microsoft Excel® 2010 was used to facilitate data handling and included in the raw data. Values below the lower limit of quantitation were entered into the data tables as LLOQ, based on a dilution factor of 1:2.

No significant hematological changes occurred. The clinical chemistry changes are outlined in TABLE 39. Intravenous administration of the modified synthetic leptin mRNA of the invention packaged with Cationic Lipid C at 0.6 mg/kg caused clinical chemistry alterations at both time points, reflecting minimal hepatocellular changes (increased Aspartate Transaminase (AST), Alanine Transaminase (ALT), decreased albumin) and potential muscle changes at 24 hours post-dose (increased AST and CK). Subtle inflammatory changes were indicated by decreased albumin (in addition to increases in pro-inflammatory cytokines detailed below). Decreased triglycerides primarily at 24 hours post-dose may have reflected an impact on lipid metabolism, decreased food intake or could be due to biological variation.

TABLE 39
Salient Clinical Chemistry Changes Associated with
the Administration of the Modified Synthetic Leptin
mRNA Packaged with Cationic Lipid B to Male Cynomolgus
Monkeys Compared to Pre-dose Values

| Analyte | 24 hours post-dose | 72 hours post-dose |
|---|---|---|
| AST | +2.7-fold | +1.2-fold (2/3 animals) |
| ALT | +1.85-fold | +2.5-fold |
| CK | 2688.5% (2/3 animals) | — |
| Albumin | −3.5% | −6.5% |
| Triglycerides | −47.6% | −47.6% |

Increased cytokine and chemokine were noted following dosing with the modified synthetic leptin mRNA of the invention packaged with Cationic Lipid B at a dose of 0.6 mg/kg intravenously, when values were compared to pre-dose values. Fold changes were calculated using the LLOQ if the analyte pre-dose values were below LLOQ.

Several of the modified synthetic leptin mRNA-related changes were noted in all three monkeys mostly at 6 hours post-dose and most of these changes returned to pre-dose levels by 24 hours post-dose. There were also a few analytes that were elevated only in one of the monkeys. The relationship of these latter changes to treatment cannot be ruled out because they were above pre-dose values and because other cytokines were increased in the same monkeys.

Increases occurred in all three monkeys in serum IL1RA levels at 6 hours post-dose, with levels returning to normal by 24 hours post-dose (the increases varied from approximately 2-fold up to approximately 11-fold). Serum IL6 levels were elevated approximately 5-fold at 3 hours post-dose in 2 of the 3 monkeys and were further increased up to approximately 4 to 8-fold in all monkeys at 6 hours post-dose returning to pre-dose values by 24 hours. Serum MCP-1 levels were increased approximately 1-fold at 1 hour post-dose in 2 of the 3 monkeys, the levels were elevated 1- to 4-fold in all animals at 3 hours post-dose, whereas at 6 hours post-dose MCP-1 levels were increased 14- to 30-fold in 2 of the 3 monkeys and 4-fold in one monkey with increases remaining 1- to 3-fold at 24 hours post-dose in all monkey. Serum levels of I-TAC were increased 4- to 37-fold in all three animals at 6 hours post-dose and remained elevated at 24 hours post-dose. TABLE 40 outlines the changes over time for the four analytes with the most consistent elevations.

TABLE 40
Salient Changes in Serum Cytokine and Chemokine Levels
Associated with the Administration of the Modified Synthetic
Leptin mRNA Packaged with Cationic Lipid B to Male Cynomolgus
Monkeys Compared to Pre-dose Values

| | 1 hour | 3 hour | 6 hour | 24 hour |
|---|---|---|---|---|
| Analyte | post-dose | post-dose | post-dose | post-dose |
| Animal 1 | | | | |
| IL1RA | —^{a} | — | 2.3x | — |
| IL6 | — | — | 4.3x | — |
| MCP-1 | 1.2x | 1.3x | 4.3x | 2.3x |
| I-TAC | — | — | 4.2x | 20.3x |
| Animal 2 | | | | |
| IL1RA | — | — | 4.6x | — |
| IL6 | — | 5.2x | 7.9x | — |
| MCP-1 | 1.2x | 2x | 14.1x | 3.6x |
| I-TAC | — | — | 10.1x | 6.3x |
| Animal 3 | | | | |
| IL1RA | — | — | 11.3x | — |
| IL6 | — | 5.6x | 8x | — |
| MCP-1 | — | 4x | 30.6x | 1.3x |
| I-TAC | — | — | 37.2x | 6.6x |

^{a}= unchanged/below baseline or value recorded as LLOQ

TABLE 40 shows the changes in serum cytokine and chemokine levels associated with the administration of the modified synthetic leptin mRNA of the invention packaged with Cationic Lipid B to male cynomolgus monkeys as compared to pre-dose values.

Under the conditions of the EXAMPLE, the modified synthetic leptin mRNA of the invention packaged with Cationic Lipid B administered intravenously at 0.6 mg/kg to male cynomolgus monkeys produced inflammatory changes based on clinical pathology parameters and increased pro-inflammatory cytokines and chemokines. Additional clinical pathology alterations suggested liver and muscle changes. There was also an increase in the anti-inflammatory cytokine, IL-1 RA. All of the cytokine changes were most prominent at 6 hours post-dose and were mostly normal by 24 hrs. Clinical pathology changes were prominent at 24 hours post-dose, but some changes persisted throughout the study.

Leptin itself has an immunomodulatory effect through increasing both pro- and anti-inflammatory cytokines (stimulating TNFα, IL-6 and IFNγ as well as IL-1RA in vitro in peripheral blood mononuclear cells. See, Juge-Aubry C E and Meier C A (2002) Mol. Cell Endocrinol. 194(1-2): 1-7).

In conclusion, a single intravenous dose of the modified synthetic leptin mRNA of the invention packaged with Cationic Lipid B administered at 0.6 mg/kg to cynomolgus monkeys was well tolerated and produced inflammatory changes and mostly minimal changes suggestive of liver and muscle damage.

### Example 24

### Pharmacodynamics of Human Leptin mRNA Packaged in Cationic Lipid B after Subcutaneous Dosing to Ob/Ob Mice

The purpose of this EXAMPLE was to demonstrate the specific effect of leptin protein on body weight and food intake in a mouse model of genetic leptin deficiency. The leptin protein was delivered by HPLC purified human leptin mRNA (SEQ ID NO: 4) in which the uridines were substituted with pseudouridine, where the mRNA was packaged in Cationic Lipid B and administered subcutaneously.

The animals used were 10 week old male ob/ob mice from Jackson Labs. The mice were single housed with a normal light cycle (6:00-18:00). They were given the HPLC purified human leptin mRNA of the invention (SEQ ID NO: 4), in which the uridines were substituted with pseudouridine, or a HPLC purified control human leptin mRNA mutated as to be non-translatable (SEQ ID NO: 16) in which the uridines were substituted with pseudouridine. The mRNA were packaged in Cationic Lipid C (N:P molar ratio=4:1). The formulation was diluted in injectable phosphate buffered saline (PBS). Mice were injected subcutaneously as described in EXAMPLE 8.

Five mice received PBS (Group A). Five mice received the modified synthetic leptin mRNA at a dose of 0.2 mpk (Group B). Five mice received the modified synthetic leptin mRNA at a dose of 0.6 mpk (Group C). Five mice received the modified synthetic leptin mRNA at a dose of 2 mpk (Group D). Five mice received the modified synthetic leptin mRNA at a dose of 6 mpk (Group E). Five mice received the modified synthetic non-translatable leptin mRNA at a dose of 0.6 mpk (Group F). Five mice received the modified synthetic non-translatable leptin mRNA at a dose of 2 mpk (Group G). Five mice received the modified synthetic leptin mRNA at a dose of 6 mpk (Group H).

On day 0 mice were weighed and sorted into groups according to their body weights so that the average body weight per group is the same, and then dosed at 9:00 AM based on their grouping. Their food was also weighed at the beginning of the study. All mice were weighed and their food is weighed at 9:00 AM on days 1, 2, 3, 4, 5, 6, 7, 10, 14 and 24.

TABLE 41 represents the body weight and TABLE 42 represents the food intake results of this EXAMPLE. The lowest dose of the leptin mRNA (0.2 mg/kg) was efficacious on body weight and food intake. The effect of leptin mRNA on body weight and food intake dose-dependently increased through the top 6 mg per kg dose. There was only a minor effect of the control non-translatable leptin mRNA at the 2 mg per kg dose. The effect of non-translatable leptin mRNA was greater at the highest 6 mg per kg dose, but this effect was still less than what was seen at the lowest dose of translatable leptin mRNA. These results demonstrate that leptin mRNA administered subcutaneously to leptin-deficient mice potently reduces food intake and body weight, and this affect is largely due to the specific expression of the human leptin protein.

TABLE 41
Effect of Human Leptin mRNA Administered by
Subcutaneous Injection on Body Weight in ob/ob Mice

| | | | | | | Non- | Non- | Non- |
|---|---|---|---|---|---|---|---|---|
| | | | | | | trans- | trans- | trans- |
| | | | | | | lat- | lat- | lat- |
| | | | | | | able | able | able |
| | | Lep- | Lep- | Lep- | Lep- | Lep- | Lep- | Lep- |
| | | tin | tin | tin | tin | tin | tin | tin |
| Day | PBS | 0.2 mpk | 0.6 mpk | 2 mpk | 6 mpk | 0.6 mpk | 2 mpk | 6 mpk |
| 1 | 0.4 | −1.4 | −1.6 | −1.7 | −1.8 | −0.2 | −1.0 | −1.6 |
| 2 | 0.2 | −3.3 | −4.1 | −4.6 | −4.5 | −0.6 | −2.1 | −3.0 |
| 3 | 0.2 | −3.8 | −5.3 | −6.1 | −6.1 | 0.0 | −2.0 | −3.1 |
| 4 | −0.5 | −5.2 | −6.7 | −7.6 | −8.0 | −0.4 | −2.1 | −3.3 |
| 5 | −0.7 | −4.8 | −7.1 | −8.9 | −9.3 | −0.2 | −2.3 | −3.2 |
| 6 | 0.8 | −4.0 | −6.3 | −8.9 | −10.0 | 1.0 | −0.9 | −2.3 |
| 7 | 0.2 | −3.9 | −6.3 | −9.1 | −11.0 | 0.4 | −1.6 | −2.4 |
| 10 | 0.3 | −2.6 | −4.8 | −7.4 | −11.1 | 0.4 | −1.2 | −2.0 |
| 14 | 1.4 | −0.6 | −2.9 | −4.7 | −7.6 | 1.9 | 0.0 | −0.5 |
| 24 | 2.9 | 1.9 | −0.4 | −0.9 | −2.0 | 4.3 | 2.1 | 1.3 |

Change in body weight in grams from baseline (day 0)

TABLE 42
Effect of Human Leptin mRNA Administered by
Subcutaneous Injection on Food Intake in ob/ob Mice

| | | | | | | Non- | Non- | Non- |
|---|---|---|---|---|---|---|---|---|
| | | | | | | trans- | trans- | trans- |
| | | | | | | lat- | lat- | lat- |
| | | | | | | able | able | able |
| | | Lep- | Lep- | Lep- | Lep- | Lep- | Lep- | Lep- |
| | | tin | tin | tin | tin | tin | tin | tin |
| Day | PBS | 0.2 mpk | 0.6 mpk | 2 mpk | 6 mpk | 0.6 mpk | 2 mpk | 6 mpk |
| 1 | 7.1 | 6.2 | 3.2 | 3.6 | 2.9 | 6.3 | 5.5 | 4.0 |
| 2 | 6.2 | 1.8 | 0.8 | 0.5 | 0.4 | 5.2 | 4.0 | 2.1 |
| 3 | 6.2 | 1.8 | 0.3 | 0.0 | −0.2 | 6.3 | 4.3 | 2.9 |
| 4 | 7.5 | 3.6 | 2.0 | 1.0 | 0.6 | 7.2 | 6.4 | 5.2 |
| 5 | 7.0 | 4.5 | 2.6 | 0.8 | 0.4 | 6.8 | 6.4 | 5.6 |
| 6 | 6.4 | 4.3 | 3.5 | 1.5 | 0.5 | 6.2 | 6.0 | 4.9 |
| 7 | 6.8 | 5.5 | 4.6 | 2.6 | 0.9 | 6.8 | 6.8 | 5.9 |
| 10 | 7.6 | 6.4 | 5.5 | 4.7 | 2.7 | 6.9 | 6.9 | 5.9 |
| 14 | 7.6 | 6.7 | 5.9 | 6.0 | 5.8 | 6.6 | 7.0 | 5.7 |

Food intake in grams per day

### Example 25

### Pharmacodynamics of Human Leptin mRNA Packaged in Cationic Lipid C after Subcutaneous Dosing to Ob/Ob Mice

The purpose of this EXAMPLE was to demonstrate the specific effect of HPLC purified human leptin mRNA (SEQ ID NO 4), in which the uridines were substituted with pseudouridine, packaged in Cationic Lipid C and administered subcutaneously on body weight and food intake in a mouse model of genetic leptin deficiency.

8 week old male leptin-deficient ob/ob mice from Jackson Labs were housed individually with a normal light cycle (6:00-18:00). They were given the HPLC purified human leptin mRNA of the invention (SEQ ID NO: 4), in which the uridines were substituted with pseudouridine, or given HPLC purified non-translatable human leptin mRNA (SEQ ID NO: 21). The mRNA were packaged in Cationic Lipid C (N:P molar ratio=4:1) according to the formulation process of EXAMPLE 35. The formulation was diluted in injectable phosphate buffered saline (PBS). Mice were injected subcutaneously as described in EXAMPLE 8.

On day 0 mice were weighed and sorted into groups according to their body weights so that the average body weight per group is the same, and then dosed at 9:00 AM based on their grouping. Groups of five mice each received either phosphate buffered saline or 0.6 milligrams per kilogram (mpk) of leptin mRNA or non-translatable leptin mRNA formulated in Cationic Lipid C. Their food was also weighed at the beginning of the study. All mice were weighed and their food is weighed at 9:00 AM on days 1, 2, 3, 4, 5, 6, 7, 10, and 15.

Body weight changes after subcutaneous administration of leptin mRNA in Cationic Lipid C are detailed in TABLE 43. Food intake is detailed in TABLE 44. The results with Cationic Lipid C demonstrate that subcutaneous administration of 0.6 mpk leptin mRNA that can lead to leptin protein expression decreases body weight and food intake in ob/ob mice. By contrast, administration of a 0.6 mpk of a non-translatable leptin mRNA has minimal effects on body weight and food intake.

TABLE 43
Effect of Human Leptin mRNA in Cationic Lipid C Administered
by Subcutaneous Injection on Body Weight in ob/ob Mice

| Time point | | Leptin mRNA | Non-translatable |
|---|---|---|---|
| (days) | PBS | 0.6 mpk | Leptin 0.6 mpk |
| 1 | −0.1 | −1.5 | 0.0 |
| 2 | 0.1 | −3.4 | −0.7 |
| 3 | 1.0 | −3.4 | −0.7 |
| 4 | 1.6 | −3.3 | −0.4 |
| 5 | 2.2 | −2.1 | −0.3 |
| 6 | 2.3 | −1.6 | 1.0 |
| 7 | 2.8 | −0.8 | 0.9 |
| 10 | 4.0 | 1.1 | 1.2 |
| 15 | 5.4 | 3.9 | 1.7 |

Change in body weight from time 0 in grams

TABLE 44
Effect of Human Leptin mRNA in Cationic Lipid C Administered
by Subcutaneous Injection on Food Intake in ob/ob Mice

| Time point | | Leptin mRNA | Non-translatable |
|---|---|---|---|
| (days) | PBS | 0.6 mpk | Leptin 0.6 mpk |
| 1 | 5.7 | 3.9 | 5.8 |
| 2 | 8.0 | 2.7 | 6.6 |
| 3 | 9.2 | 5.2 | 8.4 |
| 4 | 8.8 | 4.6 | 7.9 |
| 5 | 7.1 | 6.5 | 7.4 |
| 6 | 7.9 | 7.4 | 8.1 |
| 7 | 8.4 | 7.9 | 8.5 |
| 10 | 7.4 | 7.4 | 6.8 |
| 15 | 6.9 | 7.9 | 7.0 |

Food intake in grams

### Example 26

### Pharmacokinetics of Leptin mRNA Packaged with Cationic Lipid C after Subcutaneous Administration to Leptin-Deficient Ob/Ob Mice

The purpose of this EXAMPLE was to demonstrate the expression levels of human leptin protein after HPLC purified human leptin mRNA (SEQ ID NO: 4), in which the uridines were substituted with pseudouridine, was packaged in Cationic Lipid C and administered subcutaneously to a mouse model of genetic leptin deficiency.

The animals used were 10 week old male ob/ob mice from Jackson Labs. Animals were single housed with a normal light cycle (6:00-18:00). They were given the HPLC purified human leptin mRNA of the invention (SEQ ID NO: 4), in which the uridines were substituted with pseudouridine, or a HPLC purified control human leptin mRNA mutated as to be non-translatable (SEQ ID NO: 21), in which the uridines were substituted with pseudouridine, packaged in Cationic Lipid C (N:P molar ratio=4:1) and diluted in injectable phosphate buffered saline (PBS). Mice were injected subcutaneously as described in EXAMPLE 8.

Four animals received PBS (Group A). Four animals received the modified synthetic leptin mRNA at a dose of 0.2 mpk (Group B). Four animals received the modified synthetic leptin mRNA at a dose of 0.6 mpk (Group C). Four animals received the modified synthetic leptin mRNA at a dose of 2 mpk (Group D). Four animals received the modified synthetic leptin mRNA at a dose of 6 mpk (Group E). Four animals received the modified synthetic non-translatable leptin mRNA at a dose of 6 mpk (Group F).

On day 0 mice were weighed and sorted into groups according to their body weights, so that the average body weight per group as the same, and then dosed at 9:00 AM based on their grouping. All mice were also weighed at 9:00 AM on every subsequent day of the study.

Mice from each group were bled by tail nick on day 0 at 3:00 PM (6 hours post-dose), and then at 9:00 AM on days 1, and 2. Plasma was isolated and human leptin or mouse erythropoietin levels are measured.

TABLE 43 represents the plasma human leptin level results of this study, measured according to the protocol in EXAMPLE 1.

TABLE 45
Effect of Human Leptin mRNA Administered by Subcutaneous
Injection on Leptin Protein Expression in ob/ob Mice

| | Leptin | Leptin | Leptin | Leptin | Non-translatable |
|---|---|---|---|---|---|
| Hour | 0.2 mpk | 0.6 mpk | 2 mpk | 6 mpk | Leptin 6 mpk |
| 6 | 5537 | 6470 | 14251 | 24265 | BLQ |
| 24 | 16207 | 29776 | 90894 | 163714 | BLQ |
| 48 | 10672 | 18669 | 75844 | 131279 | BLQ |

Human leptin in pg/mL, BLQ = below limit of quantification

### Example 27

### Synthesis of Cationic Lipid A

### Preparation of Intermediate A1: (6Z,9Z)-18-bromooctadeca-6,9-diene

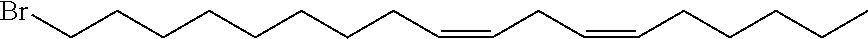 [SMILES: CCCCC/C=C\C/C=C\CCCCCCCCBr]

In a 500 mL round-bottom flask equipped with a stir bar, Linoleyl Mesylate (50 g, 145 mmol) was dissolved in diethyl ether (200 mL). Magnesium bromide diethyl etherate (101 g, 392 mmol) was added slowly. Reaction was stirred overnight at room temperature. Brine and ether were added to the mixture in a separatory funnel. The organics were then washed with brine, dried over MgSO4, filtered and concentrated under pressure to give crude product mixture. The crude was then purified by silica gel column chromatography eluting with 100% heptane to afford 45 g product. ¹H NMR (400 MHz, CDCl₃) δ=5.26-5.46 (m, 4H) 3.42 (t, J=6.90 Hz, 2H) 2.78 (t, J=6.65 Hz, 2H) 2.06 (q, J=6.78 Hz, 4H) 1.86 (dt, J=14.43, 7.09 Hz, 2H) 1.21-1.49 (m, 16H) 0.82-0.95 (m, 3H) ppm.

### Preparation of Intermediate A2: (13Z,16Z)-1-((tert-butyldimethylsilyl)oxy)docosa-13,16-dien-4-ol

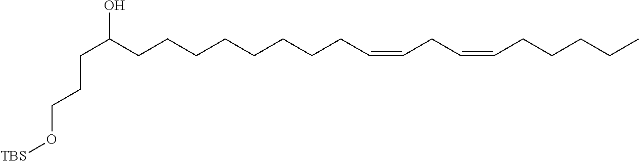

In a 500 mL three neck round-bottom flask equipped with a stir bar Mg turnings (0.443 g, 18.22 mmol) was weighed out, and held under vacuum for 5 minutes while being heated with a heat gun. The flask was left under vacuum for 5-10 minutes. The flask was then filled with N₂, and sealed with a septa. THF (75 mL) was poured quickly into the reaction vessel, followed by I₂ (0.039 g, 0.152 mmol) and Intermediate A1 (5 g, 15.18 mmol), and a reflux condenser was fitted to the flask. The whole system was exchanged with N2 three times, and the mixture was heated to reflux (90° C.) for 2 hours. The clear mixture was then cooled to room temperature under N₂.

In a second 250 mL round-bottom flask, 4-((tert-butyldimethylsilyl)oxy)butanal (2.76 g, 13.66 mmol) was dissolved in THF (30 mL) and cooled in an ice-water bath. The prepared linoleyl Grignard reagent was then added to the aldehyde dropwise over 10 minutes by syringe, and the reaction stirred at ambient temperature for 30 minutes. The reaction flask was cooled again in an ice-bath. saturated NH₄Cl (aq.) solution was added slowly to adjust pH to 7, and reaction stirred, warming to room temperature over 1 hour. Brine and ethyl acetate were added to the mixture in a separatory funnel. The organics were collected and washed with brine, dried over MgSO4, filtered and concentrated under reduced pressure to give crude product mixture. The crude was then purified by silica gel column chromatography eluting with 0-20% ethyl acetate in heptane to afford 4.91 g product as a colorless oil. ¹H NMR (400 MHz, CDCl₃) δ=5.26-5.46 (m, 4H) 3.51-3.80 (m, 3H) 2.71-2.84 (m, 2H) 1.99-2.13 (m, 4H) 1.56-1.75 (m, 4H) 1.21-1.52 (m, 20H) 0.82-0.98 (m, 12H) 0.01-0.14 (m, 6H) ppm.

### Preparation of Intermediate A3: (13Z,16Z)-1-((tert-butyldimethylsilyl)oxy)docosa-13,16-dien-4-yl (3-(dimethylamino)propyl) carbonate

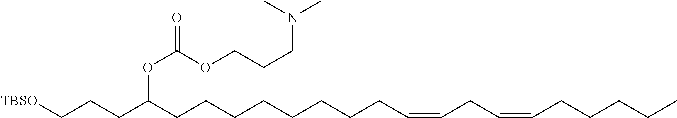 [SMILES: CCCCC/C=C\C/C=C\CCCCCCCCC(CCCO[Si](C)(C)C(C)(C)C)OC(=O)OCCCN(C)C]

In a 500 mL round-bottom flask equipped with a stir bar, 4-nitrophenylchloroformate (3.49 g, 17.31 mmol) was dissolved in DCM (100 mL). Py (1.4 mL, 17.31 mmol) and DMAP (397 mg, 3.25 mmol) were added, followed by Intermediate A2 (4.9 g, 10.82 mmol), and mixture was stirred at room temperature overnight. 3-dimethylamino-1-propanol (6.7 g, 64.9 mmol) was then added, and the mixture was then transferred to a sealed tube and heated overnight at 50° C. DCM was added to dilute the reaction mixture. Sat. NaHCO₃ (aq.) (50 mL×4) was used to wash the reaction. The organic phase was dried over MgSO4, filtered and concentrated under reduced pressure. The residue was purified by silica gel column chromatography eluting with 0-60% ethyl acetate in heptane. The isolated material was passed through a Bond Elute NH2 column (Agilent) to afford 4 g product as a colorless oil. ¹H NMR (400 MHz, CDCl₃) δ=5.27-5.47 (m, 4H) 4.62-4.79 (m, 1H) 4.19 (t, J=6.40 Hz, 2H) 3.61 (d, J=3.01 Hz, 2H) 2.72-2.83 (m, 2H) 2.42-2.59 (m, 2H) 2.31 (br. s., 6H) 2.05 (d, J=6.78 Hz, 4H) 1.83-2.00 (m, 2H) 1.47-1.74 (m, 6H) 1.17-1.43 (m, 18H) 0.80-0.97 (m, 12H) −0.05-0.10 (m, 6H) ppm.

### Preparation of Intermediate A4: 3-(dimethylamino)propyl ((13Z,16Z)-1-hydroxydocosa-13,16-dien-4-yl) carbonate

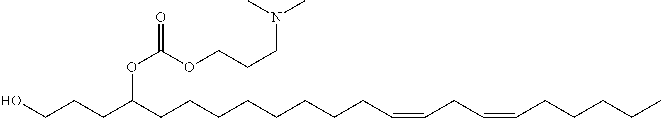 [SMILES: CCCCC/C=C\C/C=C\CCCCCCCCC(CCCO)OC(=O)OCCCN(C)C]

In a plastic tube, intermediate A3 (2 g, 3.44 mmol) was dissolved in 20 mL THF, cooled in an ice bath. HF-Py (1.51 mL, 86 mmol) was added dropwise. The mixture was then warmed up to ambient temperature and stirred for one hour. The reaction mixture was transferred to a separatory funnel and diluted with DCM. Saturated NaHCO₃ (aq.) was added slowly until the reaction mixture stopped bubbling, and the aqueous phase was separated and extracted with ethyl acetate three times. The organic phases were combined, dried over MgSO₄, filtered and concentrated under reduced pressure. The crude material was used without further purification. ¹H NMR (400 MHz, CDCl₃) δ=5.28-5.47 (m, 4H) 4.67-4.80 (m, 1H) 4.13-4.28 (m, 2H) 3.66 (t, J=5.77 Hz, 2H) 2.78 (t, J=6.53 Hz, 2H) 2.44-2.65 (m, 2H) 2.26-2.44 (m, 6H) 2.01-2.14 (m, 4H) 1.88-2.01 (m, 2H) 1.50-1.80 (m, 6H) 1.19-1.44 (m, 18H) 0.80-0.97 (m, 3H) ppm.

### Preparation of Intermediate A5: (13Z,16Z)-4-(((3-(dimethylamino)propoxy)carbonyl)oxy)docosa-13,16-dienoic acid

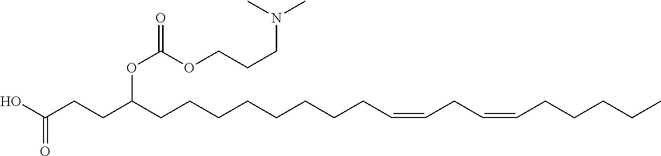 [SMILES: CCCCC/C=C\C/C=C\CCCCCCCCC(CCC(=O)O)OC(=O)OCCCN(C)C]

Jones reagent (1.6 mL, 3.2 mmol, 2 M solution in H₂O) was added dropwise to a solution of the intermediate A4 (1.5 g, 3.21 mmol) in acetone (20 mL), cooled in an ice-water bath, until the reaction mixture stayed orange for >5 min. It was then stirred for 30 minutes, after which time the cooling bath was removed, and stirring was continued for an additional 45 minutes. iPrOH (1 mL) was added, and the mixture was filtered. Acetone and 5 M aq. NaOH solution. was added until the pH was adjusted to 6. The mixture was extracted with 5% MeOH in DCM four times. The combined DCM extracts were dried and concentrated under reduced pressure to afford 288 mg product, which was used without further purification. ¹H NMR (400 MHz, CDCl₃) δ=5.25-5.48 (m, 4H) 4.84 (dt, J=7.72, 3.80 Hz, 1H) 4.38 (ddd, J=10.85, 6.84, 3.39 Hz, 1H) 3.98 (ddd, J=11.04, 8.28, 3.01 Hz, 1H) 2.99-3.10 (m, 1H) 2.78 (t, J=6.53 Hz, 2H) 2.62-2.74 (m, 2H) 2.47-2.59 (m, 6H) 2.24-2.42 (m, 2H) 2.18 (br. s., 1H) 1.89-2.11 (m, 6H) 1.58-1.78 (m, 2H) 1.47-1.58 (m, 1H) 1.19-1.42 (m, 18H) 0.89 (t, J=6.65 Hz, 3H) ppm.

### Synthesis of lipid A: 2-(((13Z,16Z)-4-(((3-(dimethylamino)propoxy)carbonyl)oxy)docosa-13,16-dienoyl)oxy)propane-1,3-diyl dioctanoate

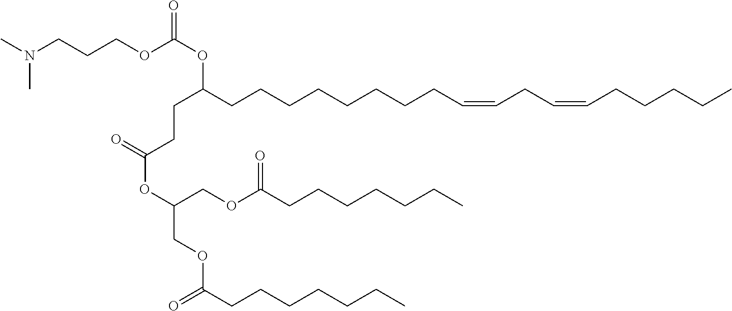 [SMILES: CCCCC/C=C\C/C=C\CCCCCCCCC(CCC(=O)OC(COC(=O)CCCCCCC)COC(=O)CCCCCCC)OC(=O)OCCCN(C)C]

In a round bottom flask, intermediate A5 (280 mg, 0.581 mmol), DMAP (28.4 mg, 0.233 mmol), EDC HCl (223 mg, 1.163 mmol) and 2-hydroxypropane-1,3-diyl dioctanoate (300 mg, 0.872 mmol) were taken into dichloromethane (5 mL). DIPEA (0.2 mL, 1.163 mmol) was added in dropwise, and the reaction was stirred at ambient temperature. After 24 hours, the reaction was concentrated under reduced pressure. The residue was purified by flash column chromatography (silica gel, 0% to 50% ethyl acetate in heptane). Fractions were collected and solvents were removed under reduced pressure to provide 188 mg product as a colorless oil. ¹H NMR (400 MHz, CDCl₃) δ=5.45-5.21 (m, 5H), 4.73 (d, J=4.3 Hz, 1H), 4.30 (dd, J=4.3, 11.8 Hz, 2H), 4.23-4.07 (m, 4H), 2.77 (t, J=6.3 Hz, 2H), 2.49-2.36 (m, 4H), 2.36-2.21 (m, 9H), 2.12-1.80 (m, 7H), 1.70-1.48 (m, 6H), 1.40-1.20 (m, 36H), 0.94-0.81 (m, 9H) ppm.

Liquid chromatography/mass spectroscopy (LC-MS m/z)=808.5 (M+1).

### Example 28

### Synthesis of Cationic Lipid B

### Preparation of Intermediate 1a: 5-(benzyloxy)pentyl methanesulfonate

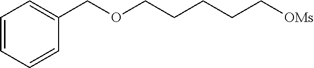 [SMILES: CS(=O)(=O)OCCCCCOCc1ccccc1]

Et₃N (10.79 mL, 78 mmol) was added in one portion by syringe to a solution of 5-Benzyloxy-1-pentanol (10.08 g, 51.9 mmol) in DCM (75 mL) in a round bottom flask charged with a magnetic stir bar at 0° C. under N₂. Next, MsCl (4.85 mL, 62.3 mmol) was added dropwise by syringe in 4 separate portions at a rate such that the internal temperature did not exceed 15° C. The reaction was allowed to continue to stir for 1 hour, after which it was diluted with H₂O (200 mL) and DCM (150 mL). The organic layer was separated, and the aqueous layer was washed with DCM (225 mL). The combined organic layers were washed with brine (200 mL), dried with Na₂SO₄, filtered, and concentrated under reduced pressure to provide the title compound as a crude orange oil (14.46 g, 99%).

Mass spectroscopy (“MS”) (m+1)=272.9.

### Preparation of Intermediate 1b: diethyl 2-(5-(benzyloxy)pentyl)malonate

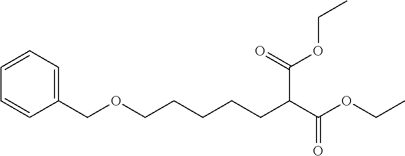 [SMILES: CCOC(=O)C(CCCCCOCc1ccccc1)C(=O)OCC]

To a cold, 0° C., solution of diethyl malonate (7.5 g, 46.8 mmol) in dry DMF (100 mL) under N₂ atmosphere was added 60% NaH (2.23 g, 56.2 mmol) portionwise over 10 minutes. The evolution of gas was observed. The mixture was stirred at 0° C. for 30 minutes then Intermediate 1a (14.46 g, 51.5 mmol) in dry DMF (41 mL) was added dropwise over 10 minutes followed by tetrabutylammonium iodide (1.73 g, 4.68 mmol). The mixture was then heated at 100° C. for 1.5 hours. The mixture was cooled at room temperature and left overnight. The mixture was then quenched with sat. NH₄Cl (50 mL) and diluted with H₂O (100 mL). The mixture was extracted with EtOAc (2×200 mL). The combined organic layers were washed with H₂O (100 mL), brine (100 mL), dried over MgSO₄, filtered, and concentrated on vacuo. The residue was purified by silica gel column chromatography eluting with 0-30% EtOAc/heptane to afford the compound as oil (11.7 g, 74%).

(MS) (m+1)=336.6.

### Preparation of Intermediate 1c: 2-(5-(benzyloxy)pentyl)propane-1,3-diol

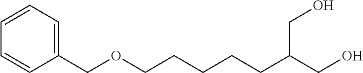 [SMILES: OCC(CO)CCCCCOCc1ccccc1]

To a cold solution, 0° C., of Intermediate 1 b (5.5 g, 16.35 mmol) in dry THF (20 mL) under N₂ was added 2.0 M LiAlH₄ in THF (24.52 mL, 49.0 mmol) dropwise over 15 min. The mixture was allowed to warm to room temperature and stirred overnight. The mixture was cooled to 0° C., treated again with 2.0 M LiAlH₄ in THF (16.35 mL, 32.7 mmol), allowed to warm to room temperature, and stirred over the weekend. The reaction was cooled to 0° C. and quenched with EtOAc (9.30 mL) dropwise over 10 min. The mixture was then treated with H₂O (3.10 mL) dropwise, a 15% NaOH solution (3.10 mL) solution dropwise, and then additional H₂O (9.30) dropwise. The mixture was stirred for 30 minutes at room temperature. The mixture was filtered through a pad of Celite. The Celite was washed with EtOAc. The filtrate was concentrated under reduced pressure to afford the title compound as a semi-wax solid (1.52 g, 37%).

MS (m+1)=253.0.

### Preparation of Intermediate 1d: 2-(5-(benzyloxy)pentyl)propane-1,3-diyl dioctanoate

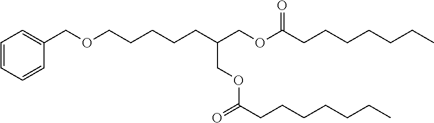 [SMILES: CCCCCCCC(=O)OCC(CCCCCOCc1ccccc1)COC(=O)CCCCCCC]

Pyridine (1.21 mL, 14.96 mmol) was added dropwise by syringe over ˜30 seconds to a solution of Intermediate 1c (1.51 g, 5.98 mmol) in DCM (20 ml) in a round bottom flask charged with a magnetic stir bar at 0° C. under N₂. Next, octanoyl chloride (2.145 mL, 12.57 mmol) was added dropwise by syringe over several minutes, and the reaction was allowed to warm to room temperature and stirred overnight. The mixture was diluted with sat. NH₄Cl (100 mL) and CH₂Cl₂ (100 mL). The organic was separated. The aqueous was extracted with CH₂Cl₂ (100 mL). The combined organics were dried over MgSO₄, filtered, and concentrated under reduced pressure. The crude material was purified by silica gel column chromatography eluting with 0-10% EtOAc/heptane to afford the title compound as colorless oil (2.88 g, 95%).

MS (m+1)=505.6.

### Preparation of Intermediate 1e: 2-(5-hydroxypentyl)propane-1,3-diyl dioctanoate

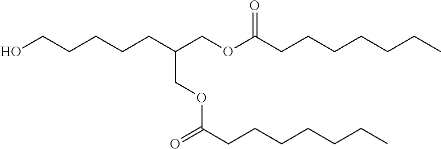 [SMILES: CCCCCCCC(=O)OCC(CCCCCO)COC(=O)CCCCCCC]

To a solution of Intermediate 1d (2.5 g, 4.95 mmol) in MeOH (25 ml) at room temperature was added 10% Pd/C, wet degussa type (264 mg). The mixture was stirred under a H₂ balloon overnight. The crude reaction mixture was filtered through a pad of Celite and filtrate was concentrated under reduced pressure to afford the title compound as colorless oil (2.0 g, 97%).

¹H NMR (400 MHz, CD₂Cl₂) δ 4.11-3.96 (m, 4H), 3.59 (t, J=6.5 Hz, 2H), 2.28 (t, J=7.5 Hz, 4H), 2.04-1.91 (m, 1H), 1.66-1.48 (m, 7H), 1.41-1.34 (m, 6H), 1.34-1.20 (m, 16H), 0.88 (t, J=6.8 Hz, 6H).

### Preparation of Intermediate 1f: 7-(octanoyloxy)-6-((octanoyloxy)methyl)heptanoic acid

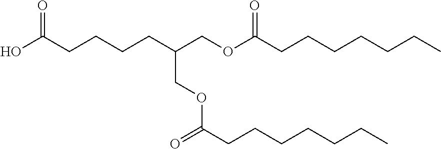 [SMILES: CCCCCCCC(=O)OCC(CCCCC(=O)O)COC(=O)CCCCCCC]

TEMPO (0.151 g, 0.965 mmol) was added in one portion to Intermediate 1e (2.0 g, 4.82 mmol) in MeCN:H₂O (46.84 mL, 1:1 ratio) in a vial charged with a magnetic stir bar at rt. Next, iodobenzene diacetate (3.42 g, 10.61 mmol) was added in one portion, and the reaction was allowed to continue to stir at room temperature overnight, after which the reaction was quenched with 15% aqueous sodium thiosulfate (50 mL). The reaction was diluted with H₂O and extracted with EtOAc (3×100 mL). The combined organic layers were dried with Na₂SO₄, filtered, and concentrated under reduced pressure to provide a pale yellow oil. The oil was dissolved in toluene (15 mL) and concentrated under reduced pressure (×6) to provide a pale yellow oil, which was dissolved in DCM and concentrated under reduced pressure (×3) to provide the title compound (plus minor aromatic impurities which could correspond to either residual toluene or iodobenzene) as a pale yellow oil (2.0 g, 97%).

MS (m-1)=427.2.

### Preparation of Intermediate 1g: 3-((tert-butyldimethylsilyl)oxy)propanal

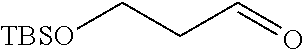 [SMILES: CC(C)(C)[Si](C)(C)OCCC=O]

In a 1 L round-bottom flask equipped with a stir bar, Tertbutyldimethylsilyloxypropanol (20 g, 105 mmol) was dissolved in DCM (500 mL). Et₃N (43.9 mL, 315 mmol) was added. In a second 500 mL flask equipped with a stir bar, SO₃.Py (25.08 g, 158 mmol) was dissolved in DMSO (100 mL, 1409 mmol). The resulting solution was added dropwise to the alcohol solution at 0° C. (in an ice-water bath). The reaction was stirred while warming to room temperature over the weekend. Water and DCM were added to the mixture in a separatory funnel. The organics were then washed with water, extracted in DCM, dried over MgSO₄, filtered and concentrated (cold) under reduced pressure to give crude product mixture. Purification by silica gel column chromatography (330 g column, 100% DCM) provided the title compound (17.7 g, 89%).

¹H NMR (400 MHz, CDCl₃) δ=9.81 (t, J=2.1 Hz, 1H), 3.99 (t, J=6.0 Hz, 2H), 2.61 (td, J=6.0, 2.3 Hz, 2H), 0.88 (s, 9H), 0.07 (s, 6H).

### Preparation of Intermediate 1h: 1-((tert-butyldimethylsilyl)oxy)pentadecan-3-ol

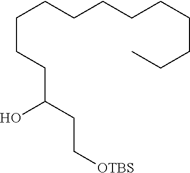 [SMILES: CCCCCCCCCCCCC(O)CCO[Si](C)(C)C(C)(C)C]

In a 500 mL round-bottom flask, Intermediate 1g (17.7 g, 94 mmol) was dissolved in THF (100 mL), and cooled to 0° C. in an ice-water bath. Dodecylmagnesiumbromide, 1M in diethyl ether (132 mL, 132 mmol) was then added to the aldehyde dropwise over 10 minutes by pipette, the ice-bath was removed, and reaction was stirred at room temperature for 30 minutes. The reaction flask was cooled again to 0° C. in an ice-bath. A saturated NH₄Cl solution was added slowly to adjust to pH ˜7 (600 mL), and the mixture was poured into a 1 L separatory funnel. The organics were then washed with saturated ammonium chloride solution, extracted in EtOAc, dried over MgSO₄, filtered and concentrated under reduced pressure to give the crude product mixture. Purification by silica gel column chromatography (330 g column, 100% Heptanes for 2 column volumes, 0% to 2% EtOAc/Heptane for 1 column volume, 2% EtOAc/Heptane for 3 column volumes, 2% to 5% EtOAc/Heptane for 1 column volume, then 5% EtOAc/Heptane for 10 column volumes) provided the title compound (24.9 g, 74%).

¹H NMR (400 MHz, CDCl₃) δ 3.97-3.87 (m, 1H), 3.87-3.77 (m, 2H), 1.69-1.60 (m, 2H), 1.57-1.36 (m, 3H), 1.36-1.19 (br, 19H), 0.93-0.84 (m, 12H), 0.09 (s, 6H).

### Preparation of Intermediate 1i: 1-((tert-butyldimethylsilyl)oxy)pentadecan-3-yl (4-nitrophenyl) carbonate

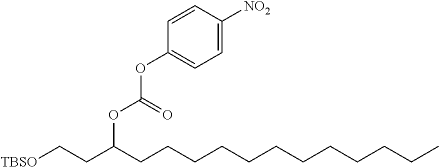 [SMILES: CCCCCCCCCCCCC(CCO[Si](C)(C)C(C)(C)C)OC(=O)Oc1ccc([N+](=O)[O-])cc1]

4-nitrophenyl carbonochloridate (2.084 g, 9.92 mmol) was added in one portion to a solution of Intermediate 1h (2.9660 g, 8.27 mmol) in DCM (28 mL) in a round bottom flask charged with a magnetic stir bar at room temperature. The reaction was fitted with a septum and placed under N₂, after which pyridine (1.003 mL, 12.40 mmol) was added dropwise by syringe over several minutes. The reaction was allowed to stir at room temperature overnight. After 24 hours of reaction time, the reaction was diluted with H₂O (100 mL) and DCM (125 mL). The organic layer was separated, and the aqueous layer was washed with DCM (125 mL). The combined organic layers were washed with brine (100 mL), dried with Na₂SO₄, filtered, and concentrated under reduced pressure to provide an off white residue. The crude residue was purified by silica gel column chromatography (80 g column, liquid loading, 0-2.5% EtOAc:heptane) to provide 3.144 g (73%) of the title compound (plus minor unidentified impurity peaks) as a colorless oil.

¹H NMR (400 MHz, CD₂Cl₂) δ 8.29-8.24 (m, 2H), 7.41-7.35 (m, 2H), 5.03-4.95 (m, 1H), 3.73 (dd, J=6.6, 5.7 Hz, 2H), 1.95-1.80 (m, 2H), 1.80-1.63 (m, 2H), 1.45-1.19 (m, 20H), 0.92-0.83 (m, 12H), 0.06 (s, 6H).

### Preparation of Intermediate 1j: 1-((tert-butyldimethylsilyl)oxy)pentadecan-3-yl (3-(diethylamino)propyl) carbonate

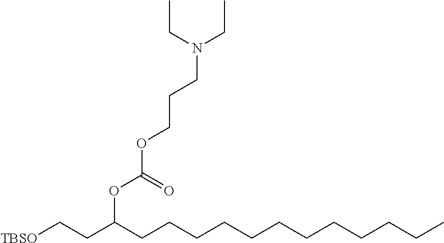 [SMILES: CCCCCCCCCCCCC(CCO[Si](C)(C)C(C)(C)C)OC(=O)OCCCN(CC)CC]

3-(diethylamino)propan-1-ol (3.48 mL, 22.26 mmol) was added dropwise by syringe over a few minutes to Intermediate 1i (2.9153 g, 5.57 mmol) in DCM (40 mL) in a round bottom flask charged with a magnetic stir bar under N₂. Next, pyridine (2.251 mL, 27.8 mmol) was added dropwise by a syringe over ˜30 seconds, followed by the addition of DMAP (0.136 g, 1.113 mmol) in one portion. The reaction was allowed to continue to stir at room temperature overnight. After 22 hours of reaction time, the reaction was diluted with H₂O (200 mL) and DCM (200 mL). The organic layer was separated and the aqueous layer was washed with DCM (200 mL). The combined organic layers were washed with brine (100 mL), dried with Na₂SO₄, filtered, and concentrated under reduced pressure to provide a crude yellow-orange oil, which was purified by silica gel column chromatography (120 g column, liquid loading (in DCM), 0-8% MeOH:DCM) to provide a yellow oil. The yellow oil was passed through a bond elut NH2 column (10 g), eluting with DCM. The filtrate was concentrated under reduced pressure to provide pale yellow oil. The bond elut procedure was repeated again to provide the title compound as colorless oil (2.0887 g, 73%).

MS (m+1)=516.4.

### Preparation of Intermediate 1k: 3-(diethylamino)propyl (1-hydroxypentadecan-3-yl) carbonate

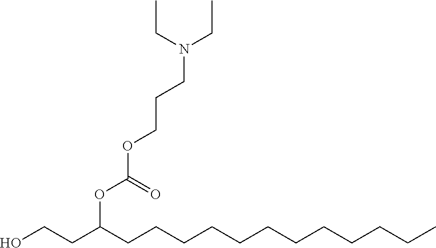 [SMILES: CCCCCCCCCCCCC(CCO)OC(=O)OCCCN(CC)CC]

To a solution of Intermediate 1j (2.2 g, 4.26 mmol) in MeOH (50 mL) at room temperature was added CAN (5.14 g, 9.38 mmol) in one portion. The mixture was stirred at room temperature and followed by TLC (70% EtOAc/heptane, KMNO₄ stain). After the consumption of starting material was observed, the mixture was diluted with CH₂Cl₂ (100 mL), and washed with sat. NaHCO₃ (2×100 mL). The aqueous layer was extracted with CH₂Cl₂ (100 mL). The combined organics were dried over Na₂SO₄, filtered, and concentrated under reduced pressure to afford the title compound (along with some minor impurities) as oil.

MS (m+1)=402.2.

### Preparation of final compound, Cationic Lipid B: 2-(10-dodecyl-3-ethyl-8,14-dioxo-7,9,13-trioxa-3-azaoctadecan-18-yl)propane-1,3-diyldioctanoate

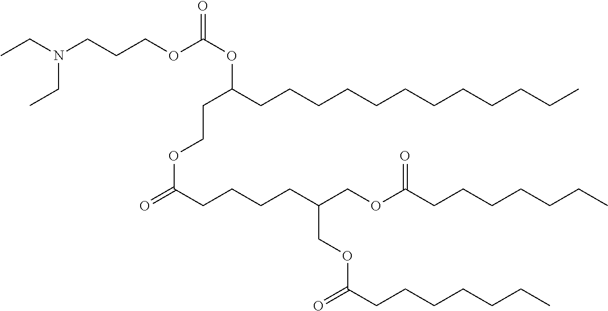 [SMILES: CCCCCCCCCCCCC(CCOC(=O)CCCCC(COC(=O)CCCCCCC)COC(=O)CCCCCCC)OC(=O)OCCCN(CC)CC]

In a round bottom flask, Intermediate 1f (1.99 g, 4.66 mmol), DMAP (0.207 g, 1.693 mmol), DIPEA (1.48 mL, 8.47 mmol), and Intermediate 1k (1.7 g, 13.07 mmol) were taken into dichloromethane (20 mL). EDC.HCl (1.62 g, 8.47 mmol) was added in one portion, and the reaction was stirred at ambient temperature. After 24h, the reaction was concentrated under reduced pressure. The residue was purified by silica gel column chromatography (0% to 5% MeOH in CH₂Cl₂). Fractions were collected and solvents were removed under reduced pressure to provide yellow oil (3.8 g). The oil was repurified by silica gel column chromatography (0-75% EtOAc/heptane) to afford the title compound as an off-white oil (2.6 g, 71.8%).

¹H NMR (400 MHz, CDCl₃) δ=4.80 (t, J=6.1 Hz, 1H), 4.25-3.96 (m, 8H), 2.62 (br. s., 4H), 2.30 (t, J=7.5 Hz, 6H), 2.05-1.85 (m, 5H), 1.71-1.53 (m, 8H), 1.43-1.21 (m, 42H), 1.21-0.98 (m, 6H), 0.96-0.79 (m, 9H). ¹³C NMR (101 MHz, CDCl₃) δ=174.15 (s, 2C), 173.71, 155.20, 75.90, 66.41, 64.14 (s, 2C), 60.85, 49.32, 47.10 (s, 2C), 37.46, 34.56 (s, 3C), 34.52, 34.29, 33.28, 32.22, 31.97 (s, 2C), 29.95 (s, 2C), 29.88, 29.80, 29.76, 29.66, 29.41 (s, 2C), 29.23 (s, 2C), 28.20, 26.64, 25.36, 25.25 (s, 4C), 23.00, 22.90 (s, 2C), 14.44, 14.38 (s, 2C), 11.37 (s, 2C).

### Example 29

### Synthesis of Cationic Lipid C

### Preparation of Intermediate 1a: (9Z,9′Z,12Z,12′Z)-2-(hydroxymethyl)propane-1,3-diyl bis(octadeca-9,12-dienoate)

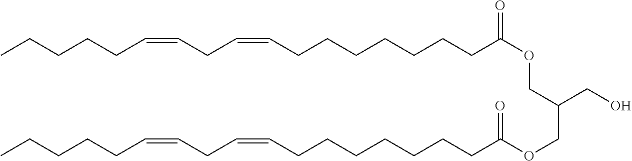 [SMILES: CCCCC/C=C\C/C=C\CCCCCCCC(=O)OCC(CO)COC(=O)CCCCCCC/C=C\C/C=C\CCCCC]

In a round bottom flask, linoleic acid (95.0 g, 339 mmol), DMAP (4.14 g, 33.90 mmol), DIPEA (74.1 mL, 424 mmol), and 2-(hydroxymethyl)propane-1,3-diol (18.0 g, 170 mmol) were taken into dichloromethane (435 ml). EDC (81.0 g, 424 mmol) was added in one portion, and the reaction was stirred at ambient temperature. After 24 hours, the reaction is concentrated under reduced pressure with silica gel powder for dry loading and the residue was purified on silica gel (Biotage) using ethyl acetate/heptane (0% to 40%) as eluent, to provide 47 g (44% yield) of the desired product as a colorless oil.

¹H NMR (400 MHz, CDCl₃): δ=5.19-5.50 (m, 8H), 4.19 (tt, J=11.83, 5.87 Hz, 4H), 3.51-3.69 (m, 2H), 2.78 (t, J=6.53 Hz, 4H), 2.33 (t, J=7.53 Hz, 4H), 2.20 (quint, J=5.83 Hz, 2H), 2.06 (q, J=6.78 Hz, 8H), 1.49-1.72 (m, 5H), 1.20-1.46 (m, 26H), 0.79-0.98 (m, 6H) ppm.

### Preparation of final compound, Cationic Lipid C: (9Z,9′Z,12Z,12′Z)-2-(((1,3-dimethylpyrrolidine-3-carbonyl)oxy)methyl)propane-1,3-diyl bis(octadeca-9,12-dienoate)

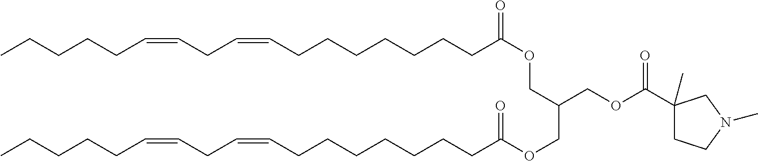 [SMILES: CCCCC/C=C\C/C=C\CCCCCCCC(=O)OCC(COC(=O)CCCCCCC/C=C\C/C=C\CCCCC)COC(=O)C1(C)CCN(C)C1]

In a round bottom flask, Intermediate 1a (15.0 g, 23.77 mmol), DMAP (0.581 g, 4.75 mmol), DIPEA (8.30 mL, 47.5 mmol) and 1,3-dimethylpyrrolidine-3-carboxylic acid (5.11 g, 35.7 mmol) were taken into dichloromethane (78 mL). EDC (9.11 mg, 47.5 mmol) was added in one portion, and the reaction was stirred at ambient temperature. After 24 hours, the reaction was concentrated under reduced pressure with dry silica gel for dry loading and the residue was purified on silica gel (Biotage) using ethyl acetate/heptane (0% to 80%) as eluent to provide 15 g (82% yield) of the desired product as a colorless oil.

¹H NMR (400 MHz, CDCl₃): δ=5.26-5.47 (m, 8H), 4.08-4.21 (m, 6H), 2.97 (d, J=9.29 Hz, 1H), 2.78 (t, J=6.53 Hz, 4H), 2.56-2.69 (m, 2H), 2.24-2.49 (m, 10H), 2.05 (q, J=6.78 Hz, 8H), 1.54-1.76 (m, 5H), 1.22-1.42 (m, 31H), 0.89 (t, J=6.78 Hz, 6H) ppm. MS (m+1)=756.7.

### Example 30

### Synthesis of Cationic Lipid D

### Preparation of Intermediate 13a: 4,4-bis(octyloxy)butanenitrile

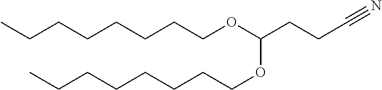 [SMILES: CCCCCCCCOC(CCC#N)OCCCCCCCC]

To a mixture of 4,4-diethoxybutanenitrile (15 g, 95 mmol) and octanol (37.3 g, 286 mmol) was added pyridinium p-toluenesulfonate (1.2 g, 4.77 mmol) and the mixture was heated to 105° C. After 72 hours, the reaction mixture is cooled and purified on silica gel using ethyl acetate/heptane as eluent to provide 9.34 g of the expected product. ¹H NMR (400 MHz, CDCl₃): δ=4.56 (t, J=5.40 Hz, 1H), 3.61 (dt, J=9.16, 6.59 Hz, 2H), 3.44 (dt, J=9.22, 6.68 Hz, 2H), 2.43 (t, J=7.28 Hz, 2H), 1.95 (td, J=7.34, 5.40 Hz, 2H), 1.50-1.66 (m, 4H), 1.17-1.44 (m, 20H), 0.80-0.95 (m, 6H) ppm.

### Preparation of Intermediate 13b: 4,4-bis(octyloxy)butanoic acid

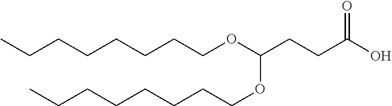 [SMILES: CCCCCCCCOC(CCC(=O)O)OCCCCCCCC]

In a high pressure reaction vessel, Intermediate 13a (9.34 g, 28.7 mmol) is dissolved in 30 mL EtOH. KOH (4.83 g) is dissolved in 30 mL water and the KOH solution was added to the EtOH solution. The tube was sealed and heated to 110° C. overnight. The mixture was cooled and diluted with EtOAc. 1N HCl was added to adjust pH to 5, and the aqueous phase was extracted with EtOAc twice. The combined organic extracts were dried over MgSO₄, filtered and concentrated under reduced pressure to provide 10.9 g of the expected product. ¹H NMR (400 MHz, CDCl₃): δ=4.46 (t, J=5.52 Hz, 1H), 3.46-3.59 (m, 2H), 3.08-3.46 (m, 3H), 2.18 (t, J=7.28 Hz, 2H), 1.72-1.89 (m, 2H), 1.46-1.63 (m, 4H), 1.28 (d, J=3.76 Hz, 20H), 0.79-0.96 (m, 6H) ppm.

### Preparation of Intermediate 13c: (9Z,12Z)-3-hydroxy-2-(hydroxymethyl)propyl octadeca-9,12-dienoate

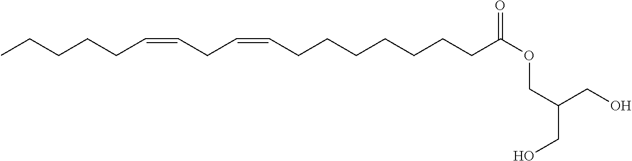 [SMILES: CCCCC/C=C\C/C=C\CCCCCCCC(=O)OCC(CO)CO]

In a round bottom flask, linoleic acid (23.78 g, 85 mmol), DMAP (2.072 g, 16.96 mmol), DIPEA (22.22 ml, 127 mmol), and 2-(hydroxymethyl)propane-1,3-diol (9 g, 85 mmol) were taken into dichloromethane (200 mL). EDC (24.39 g, 127 mmol) was added in one portion, and the reaction was stirred at ambient temperature. After 24h, the reaction is concentrated under reduced pressure, and the concentrate is purified on silica gel with ethyl acetate/heptane as eluent to provide 12.4 g of the expected product. 9.5 g of Intermediate 1a was also isolated. ¹H NMR (400 MHz, CDCl₃): δ=4.27 (d, J=6.27 Hz, 2H), 3.77 (qd, J=11.25, 5.14 Hz, 4H), 2.78 (t, J=6.40 Hz, 2H), 2.23-2.48 (m, 4H), 1.90-2.15 (m, 6H), 1.53-1.76 (m, 3H), 1.15-1.45 (m, 14H), 0.77-0.98 (m, 3H) ppm.

### Preparation of Intermediate 13d: (9Z,12Z)-3-((4,4-bis(octyloxy)butanoyl)oxy)-2-(hydroxymethyl)propyl octadeca-9,12-dienoate

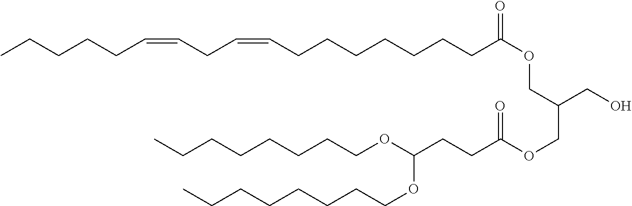 [SMILES: CCCCC/C=C\C/C=C\CCCCCCCC(=O)OCC(CO)COC(=O)CCC(OCCCCCCCC)OCCCCCCCC]

In a round bottom flask, Intermediate 13c (10.9 g, 31.6 mmol), DMAP (773 mg, 6.33 mmol), DIPEA (11.05 mL, 63.3 mmol), and Intermediate 13b (13.99 g, 38 mmol) were taken into dichloromethane (100 mL). EDC (12.13 g, 63.3 mmol) was added in one portion, and the reaction was stirred at ambient temperature. After 24h, the reaction was concentrated under reduced pressure. The concentrate was purified on silica gel with 0-20% EtOAc/heptane as eluent to provide 11.2 g of the expected product. ¹H NMR (400 MHz, CDCl₃): δ=5.27-5.45 (m, 4H), 4.50 (t, J=5.52 Hz, 1H), 4.08-4.25 (m, 4H), 3.50-3.69 (m, 4H), 3.41 (dt, J=9.22, 6.68 Hz, 2H), 2.78 (t, J=6.53 Hz, 2H), 2.42 (t, J=7.53 Hz, 2H), 2.33 (t, J=7.53 Hz, 2H), 2.13-2.29 (m, 2H), 2.00-2.13 (m, 4H), 1.88-2.00 (m, 2H), 1.49-1.69 (m, 7H), 1.20-1.44 (m, 32H), 0.83-0.95 (m, 9H) ppm.

### Preparation of final compound, Cationic Lipid D: (9Z,12Z)-3-((4,4-bis(octyloxy)butanoyl)oxy)-2-(((((1-ethylpiperidin-3-yl)methoxy)carbonyl)oxy)methyl)propyl octadeca-9,12-dienoate

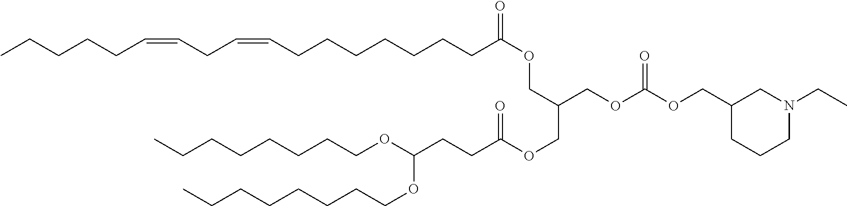 [SMILES: CCCCC/C=C\C/C=C\CCCCCCCC(=O)OCC(COC(=O)CCC(OCCCCCCCC)OCCCCCCCC)COC(=O)OCC1CCCN(CC)C1]

In a round-bottom flask, 4-nitrophenylchloroformate (290 mg, 1.439 mmol) was dissolved in DCM (10 mL). Pyridine (0.116 ml, 1.439 mmol) and DMAP (26.4 mg, 0.216 mmol) were added, followed by Intermediate 13d ((500 mg, 0.719 mmol), and mixture was stirred at ambient temperature for 1 hour. (1-ethylpiperidin-3-yl)methanol (618 mg, 4.32 mmol) was added, and the reaction was continued overnight. The reaction was concentrated under reduced pressure, and the concentrate was purified on silica gel with ethyl acetate/heptane as eluent to provide 530 mg of the expected compound. ¹H NMR (400 MHz, CDCl₃): δ=5.25-5.46 (m, 4H), 4.49 (t, J=5.52 Hz, 1H), 4.17 (dd, J=19.32, 5.77 Hz, 6H), 4.02-4.09 (m, 1H), 3.91-4.00 (m, 1H), 3.57 (dt, J=9.22, 6.68 Hz, 2H), 3.40 (dt, J=9.22, 6.68 Hz, 2H), 2.82-2.99 (m, 2H), 2.77 (t, J=6.40 Hz, 2H), 2.36-2.48 (m, 5H), 2.31 (t, J=7.53 Hz, 2H), 2.05 (q, J=6.78 Hz, 5H), 1.84-1.97 (m, 3H), 1.50-1.81 (m, 12H), 1.21-1.41 (m, 31H), 0.94-1.14 (m, 5H), 0.80-0.94 (m, 9H) ppm. MS (m+1)=865.1.

### Example 31

### Expression of Leptin Protein from Different Leptin mRNA Sequences

The purpose of this EXAMPLE was to demonstrate the influence of leptin mRNA nucleotide sequence on the efficiency of leptin protein expression.

The leptin mRNA constructs used in the EXAMPLE each contained different human leptin coding region sequences. The human leptin mRNAs of SEQ ID NO: 4, SEQ ID NO: 6 and SEQ ID NO:8 contained codon optimized sequences (SEQ ID NO: 17, SEQ ID NO: 18 and SEQ ID NO: 19, respectively). The human leptin mRNA of SEQ ID NO: 10 contains native human coding sequence (SEQ ID NO: 20). The nucleotide sequences of the four human leptin codon sequences are homologous sequences, being between 78% and 91% identical to each other.

Leptin mRNAs were transcribed as in EXAMPLE 2. HepG2/C3a cells were grown in Dulbecco's modified Eagle's medium supplemented with 10% fetal bovine serum in a 37° C. incubator with a humidified atmosphere of 5% CO₂. 1.5 micrograms of leptin mRNA was complexed with Lipofectamine 2000 (Life Technologies) and transfected into HepG2/C3a cells according to the manufacturer's protocol.

The cells were incubated for 24 hours, and condition medium was removed. The cells were washed with phosphate buffered saline once, and fresh Dulbecco's modified Eagle's medium with 10% fetal bovine serum was added to the cells. The cells were incubated for another 24 hours, and the condition medium was then removed.

Leptin protein levels in the condition medium were measured as described in EXAMPLE 1. The results are shown in TABLE 46. Leptin mRNA of SEQ ID NO: 4 supported the highest expression of leptin protein.

TABLE 46
Effect of Different Leptin mRNA Codon Optimizations
on Leptin Protein Levels in vitro

| Sequence | Leptin protein (pg/mL) |
|---|---|
| SEQ ID NO: 4 | 167,518 |
| SEQ ID NO: 6 | 58,878 |
| SEQ ID NO: 8 | 124,638 |
| SEQ ID NO: 10 | 17,238 |

### Example 32

### Leptin mRNA Packaged in Cationic Lipid C

### In Vivo Pharmacokinetic Study of Different Formulations Delivered Subcutaneously

Leptin protein expression was induced following subcutaneous delivery of human leptin mRNA (SEQ. ID: NO. 4), in which the uridines were substituted with pseudouridine, packaged in Cationic Lipid C using different lipid formulations.

Leptin mRNA was synthesized as described in EXAMPLE 2 and encapsulated in cationic lipid nanoparticles as in EXAMPLE 5, except the ratios of Cationic Lipid C to neutral lipid (DSPC), cholesterol, and lipidated PEG were varied as in TABLE 47.

TABLE 47
Leptin Protein levels in C57Bl/6 Mice Dosed Subcutaneously
with Leptin mRNA in Cationic Lipid C Formulations

| Formulation (cationic | Leptin protein |
|---|---|
| lipid:DSPC:cholesterol:PEG) | (pg/mL) |
| 40:10:48:2 | 986 |
| 30:10:58:2 | 2,142 |
| 50:10:38:2 | 2,690 |
| 45:5:48:2 | 3,793 |

9 week old male C57BI/6 mice (n=4 per group) were administered leptin mRNA in lipid nanoparticles subcutaneously as in EXAMPLE 8. On day 0, animals were weighed and sorted according to average body weight. Mice were dosed at 9 AM and blood was taken at 6 hours. Body weight and food intake were also recorded.

Human leptin protein levels were measured according to the protocol in EXAMPLE 1. Leptin protein levels after subcutaneous administration of leptin mRNA in Cationic Lipid C under different formulation conditions are detailed in TABLE 47.

Thus, subcutaneous delivery of human leptin mRNA to lean mice according to the procedure of this EXAMPLE can increase the potency of mRNA delivery after subcutaneous administration as evidenced by higher plasma human leptin protein levels.

### Example 33

### Leptin mRNA Packaged in Cationic Lipid B or Cationic Lipid C

### In Vivo Pharmacokinetic Study of Repeated Delivery Intravenously

Leptin protein expression was induced following intravenous delivery of human leptin mRNA (SEQ. ID. NO: 4) in which the uridines were substituted with pseudouridine and the leptin mRNA was, packaged in Cationic Lipid C using different lipid formulations. Leptin mRNA was synthesized as described in EXAMPLE 2 and encapsulated in cationic lipid nanoparticles as in EXAMPLE 5.

15 week old female 129Sv mice (n=4 per group) were administered leptin mRNA in lipid nanoparticles intravenously as in EXAMPLE 8. On day 0, animals were weighed and sorted according to average body weight. Mice were dosed at 9 AM on the days indicated in TABLE 48 and blood was taken at 6 hours. Body weight was also recorded.

Human leptin protein levels were measured according to the protocol in EXAMPLE 1. Leptin protein levels after each intravenous administration of leptin mRNA in Cationic Lipid B or Cationic Lipid C are detailed in TABLE 48.

TABLE 48
Leptin Protein Levels in 129Sv Mice Dosed by IV Repeatedly with
2 mg/kg Leptin mRNA in Cationic Lipid B or Cationic Lipid C

| Day of administration | Cationic Lipid B | Cationic Lipid C |
|---|---|---|
| 0 | 532,410 | 192,882 |
| 3 | 327,559 | 159,720 |
| 7 | 129,951 | 105,356 |
| 10 | 298,491 | 172,631 |
| 14 | 223,754 | 117,535 |
| 17 | 351,181 | 185,511 |

Plasma leptin protein levels in pg/mL

Repeated intravenous delivery of human leptin mRNA to 129Sv mice according to the procedure of this EXAMPLE results in largely reproducible levels of leptin protein in plasma 6 hours after mRNA delivery. For Cationic Lipid B, administration of a single 2 mg/kg dose of leptin mRNA to an additional group of mice at the same time as the repeated dose mice group received their day 17 dose resulted in plasma leptin protein levels of 252,622 μg/mL. This indicates that the variation in plasma leptin protein levels after repeated doses of leptin mRNA in Cationic Lipid B is within the normal variation for a single administration. There was no consistent difference in body weights between groups of mice over the course of the study, indicating that repeated administration of leptin mRNA in Cationic Lipid B or Cationic Lipid C was generally tolerated.

### Example 34

### Improvement Process A

mRNA Encapsulation in Lipid Nanoparticles by Improvement Process A.

The following improvement is a method is for an encapsulation process using a 0.75 ml volume of lipids and a 0.75 ml volume of mRNA at a concentration of up to 0.4 mg/mL, for a 0.3 mg mRNA encapsulation run. This process can be scaled down to as little as 40 μg of mRNA. The initial mRNA lipid nanoparticles in 50% ethanol are immediately diluted by mixing with citrate buffer in a collection vessel with a stir bar on a stir plate.

TABLE 49
Exemplary Materials and Reagents for mRNA
Encapsulation in Lipid Nanoparticles

| | | Catalog or |
|---|---|---|
| | | identification |
| Item | Vendor | number |
| Cationic lipid | Novartis | Example: Cationic |
| | | Lipid C |
| 1,2-distearoyl-sn-glycero-3- | Corden | LP-R4-076 |
| phosphocholine (DSPC) | | |
| Cholesterol | Sigma | C8667 |
| Lipidated polyethylene glycol | Novartis | Example: S024 |
| (PEG lipid) | | |
| Ethanol | Sigma | 459844 |
| Nuclease-free water | Life Technologies | 10977 |
| 100 mM citrate buffer, pH 6.0 | Teknova | Q2446 |
| Amicon Ultra-15 centrifugal | Millipore | UFC903024 |
| filter unit, 30 kDa molecular | | |
| weight cut-off | | |
| RNaseZap | Life Technologies | AM9780 |
| Syringe pump | KD Scientific | KDS200 |
| 10X phosphate buffered | Lonza | 51226 |
| saline | | |
| Minimate TFF system, 110 | PALL Corporation | OAPMP110 |
| volts | | |
| Vivaflow 50, 100 kDa | Sartorius | VF05C4 |
| molecular weight cut-off, | | |
| regenerated cellulose | | |
| Quant-IT Ribogreen RNA | Life Technologies | R11490 |
| assay kit | | |
| Tris-EDTA buffer | Promega | V6231 |
| Triton X-100 | Sigma | T8787 |
| Zetasizer NanoZS | Malvern | ZEN3600 |
| Masterflex silicone L/S 14 | Cole-Parmer | 96410-14 |
| tubing | | |
| 60 ml sterile Plastipak | Becton Dickson | 309653 |
| syringe | | |
| 140 ml sterile syringe | Tyco | 8881114030 |

Optionally, equipment and disposable supplies can be certified free of RNase activity by the manufacturer or rendered RNase free by use of the RNaseZap reagent.

The modified synthetic leptin mRNA of the invention is prepared in a citrate buffer which can be at pH 6.0, but which can also be lowered in small increments through pH 5.6 if desired.

Preparation of Lipid Mixture in Ethanol.

The following is one example of mRNA encapsulated at a cationic lipid amine to mRNA phosphate (N:P) molar ratio of 4:1. Lipids (cationic lipid, DSPC, cholesterol and lipidated PEG) are weighed out separately and prepared as 10 mg/mL stock solutions in ethanol (with the exception of polyethylene glycol (PEG), which is prepared at a concentration of 5 mg/mL). These stock solutions are used to prepare the lipid mixture. The molar ratios are 40:10:48:2, respectively. For example, in TABLE 50 are the amounts and final concentrations of all components for a 1.5 mL volume of lipids in ethanol. The 1.5 mL volume represents 2× of the required volume to ensure the target volume is available for loading into syringe. The mixture is prepared in a scintillation vial and is maintained at 37° C. until use.

TABLE 50
Lipid Mixture Example for Improvement Process A

| | | Final | |
|---|---|---|---|
| | Amount | concentration | Volume |
| Reagent | (mg) | (mM) | (μL) |
| Cationic Lipid C | 5.44 | 4.8 | 544 |
| DSPC | 1.42 | 1.2 | 142 |
| Cholesterol | 3.34 | 5.76 | 334 |
| PEG lipid (S024) | 0.97 | 0.24 | 194 |
| Ethanol | | | 285 |

Preparation of mRNA in Citrate Buffer.

The pH of the citrate buffer is first confirmed to be pH 6.0. If it is not, then the pH is adjusted before proceeding.

Enough mRNA in water for the encapsulation is thawed from −80° C. storage and exchanged from water into citrate buffer pH 6.0 by use of Amicon Ultra-15 centrifugal concentrators. Between 10 and 12 mg of mRNA can be loaded into each concentrator, which is centrifuged for 8 minutes at 4,000 rpm at 4° C. The volume is increased by the addition of citrate buffer pH 6.0. An exchange of ≧10 volumes with citrate buffer pH 6.0 is recommended to achieve the desired buffer condition. The concentration of the mRNA is measured by optical density at 260 nm in a UV spectrophotometer. The final concentration of the mRNA is adjusted to 0.4 mg/mL in 0.825 mL of citrate buffer in a rinsed scintillation vial and is held at room temperature until use. The 0.825 mL volume represents 1.1× of the required volume to ensure the target volume is available for loading into syringe.

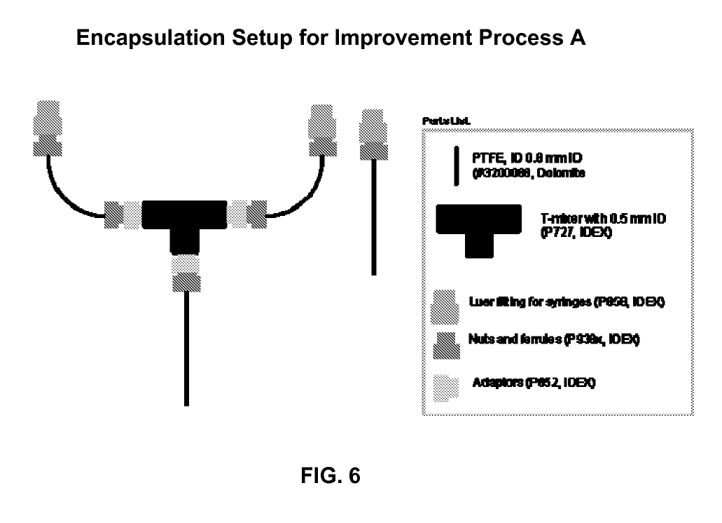

Encapsulation of mRNA in lipid nanoparticles. The T-junction with input and output lines is prepared as shown in FIG. 6. Sterile 1 mL syringes are loaded with an equal volume (0.75 mL) of lipids in ethanol (syringe (a)), mRNA in citrate buffer (syringe (b)), and citrate buffer alone (syringe (c)). Tubing leading from Luer fittings on the syringes (a) and (b) containing lipids and mRNA, respectively, are attached to the T junction, and tubing leading from a Luer fitting on syringe (c) containing citrate buffer alone is paired with the tubing exiting the T-junction over a collection vessel containing a stir bar on an active stir plate. Syringes are installed on a syringe pump. The syringe pump is set to the appropriate syringe (e.g., Becton Dickinson, Plastipak, 1 mL) and a flow rate of 1 mL per minute.

The mRNA now encapsulated in lipid nanoparticles is immediately transferred to dialysis tubing (e.g., SnakeSkin 10 kDa molecular weight cut-off, Thermo Scientific). The material is dialyzed against RNase and pyrogen-free 1× phosphate buffered saline overnight at 4° C. Remove mRNA lipid nanoparticles and place in sterile tubes and store at 4° C. until analysis.

See EXAMPLE 5 for additional information about Improvement Process A.

### Example 35

### Improvement Process B

mRNA encapsulation in lipid nanoparticles by Improvement Process B. The following improvement is a method for an encapsulation process using a 60 ml volume of lipids and a 60 ml volume of mRNA at a concentration of up to 0.5 mg/mL, or for a 30 mg mRNA encapsulation run. This process can be scaled down to as little as 2 mg of mRNA.

In contrast to Improvement Process A, in Improvement Process B the initial mRNA lipid nanoparticles in 50% ethanol are immediately diluted by mixing with citrate buffer in a second T junction before collection in a vessel. The resulting mRNA lipid nanoparticle suspension is then diluted a second time by mixing with citrate buffer through another T junction. Process B also includes the use of tangential flow filtration (TFF) for concentration of the mRNA lipid nanoparticles and buffer exchange in the TFF unit instead of dialysis. Optionally, TFF cartridges (e.g., Vivaflow 50) may first be rendered pyrogen-free.

Preparation of Lipid Mixture in Ethanol.

The following is an example of mRNA encapsulated at a cationic lipid amine to mRNA phosphate (N:P) molar ratio of 4:1. Lipids (cationic lipid, DSPC, cholesterol and lipidated PEG) are dissolved in ethanol. The molar ratios are 40:10:48:2, respectively. TABLE 51 shows the amounts and final concentrations of all components for a 63 ml volume of lipids in ethanol. The 63 mL volume represents 1.05× of the required volume to ensure the target volume is available for loading into syringe. The mixture is weighed out and placed into a sterile polyethylene terephthalate glycol-modified (PETG) 125 mL bottle and ethanol is added. The mixture is sonicated briefly, then gently agitated for 5 minutes and then maintained at 37° C. until use.

TABLE 51
Lipid Mixture Example for Improvement Process B

| | | Final |
|---|---|---|
| | Amount | concentration |
| Reagent | (mg) | (mM) |
| Cationic lipid C | 285.82 | 6.0 |
| DSPC | 74.67 | 1.5 |
| Cholesterol | 175.38 | 7.2 |
| PEG lipid (S024) | 51.03 | 0.3 |

Preparation of mRNA in Citrate Buffer.

The pH of the citrate buffer is first confirmed to be pH 6.0. If it is not, then the pH is adjusted before proceeding. Enough mRNA in water for the encapsulation is thawed from −80° C. storage and exchanged from water into citrate buffer pH 6.0 by use of Amicon Ultra-15 centrifugal concentrators. Between 10 and 12 mg of mRNA can be loaded into each concentrator, which is centrifuged for 5 minutes at 4,000 rpm at 4° C. Volume is increased by the addition of citrate buffer pH 6.0. An exchange of >10 volumes with citrate buffer pH 6.0 is recommended to achieve the desired buffer condition. The concentration of the mRNA is measured by optical density at 260 nm in a UV spectrophotometer. The final concentration of the mRNA is adjusted to 0.5 mg/mL in 63 mL of citrate buffer in a sterile 125 mL PETG bottle and is held at room temperature until use. The 63 mL volume represents 1.05× of the required volume to ensure the target volume is available for loading into syringe.

Encapsulation of mRNA in Lipid Nanoparticles.

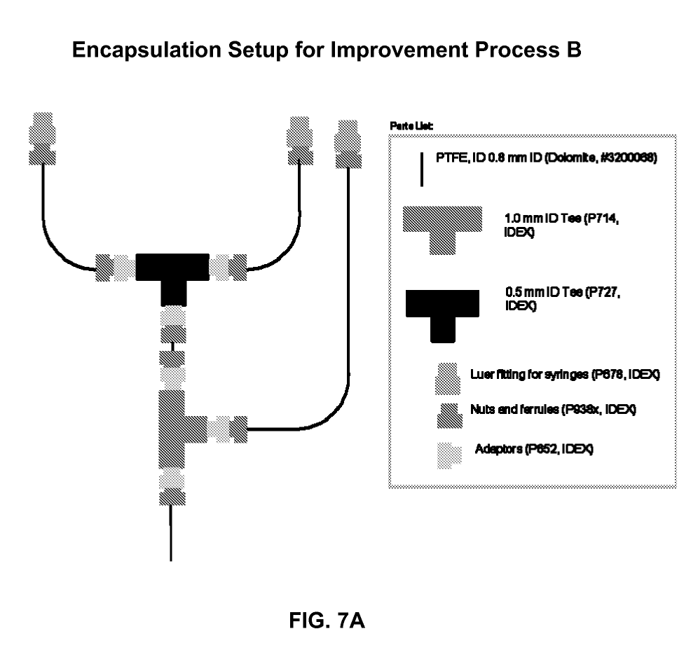

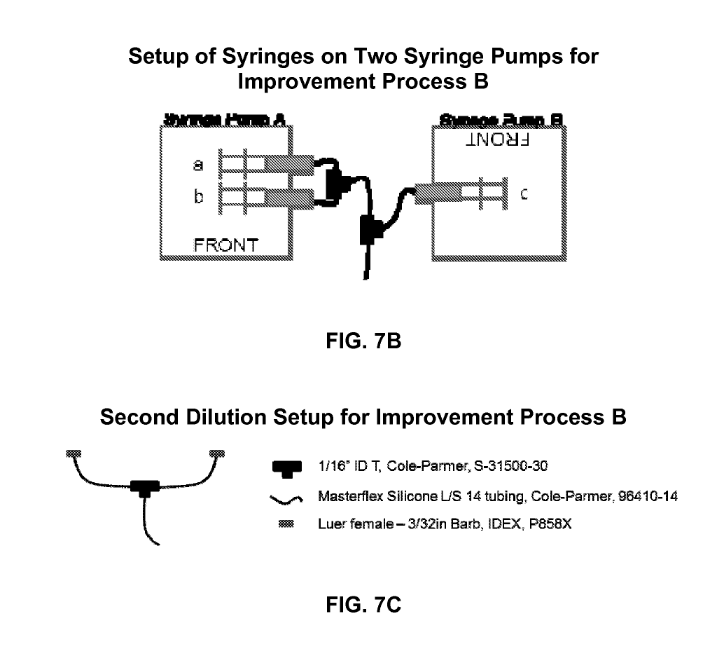

The T-junctions with input and output lines are prepared as shown in FIG. 7A. Sterile 60 mL syringes are loaded with an equal volume (60 ml) of lipids in ethanol (syringe (a)), mRNA in citrate buffer (syringe (b)), and citrate buffer alone (syringe (c)). Tubing leading from Luer fittings on the syringes (a) and (b) containing lipids and mRNA, respectively, are attached to the first T junction and installed on syringe pump A. See, FIG. 7B. Tubing leading from a Luer fitting on syringe (c) containing citrate buffer alone is attached to the second T junction to enable in-line dilution of mRNA lipid nanoparticles, and syringe (c) is installed on syringe pump B. The output line from the second T junction is positioned over a sterile 250 mL PETG bottle for collection of the diluted mRNA lipid nanoparticles. All fittings are tight on the syringes. The syringe pumps are set to the appropriate syringe manufacturer and size (BD, Plastipak, 60 ml) and a flow rate of up to 40 ml per minute. Both pumps are started simultaneously, and collecting material is started after approximately 0.5 seconds. Approximately 160-170 ml of mRNA lipid nanoparticles can be collected.

A 140 mL syringe can be used to aspirate 135 mL of the mRNA lipid nanoparticle suspension. Transfer the remaining 35-45 mL to a 60 mL syringe. Prepare a 140 mL and 60 mL syringe with the same volumes of citrate buffer. Another T junction for is set up as shown in FIG. 7C for another dilution of the mRNA lipid nanoparticle suspension. This dilution step is run with both syringes on only one syringe pump. The 140 mL syringes (one containing lipid nanoparticles, the other containing citrate buffer) are run first, and the 60 mL syringes are run second. All fittings are tight on the syringes. For the first run, the settings are changed on the syringe pump to the correct size and manufacturer (140 mL, Sherwood-monoject). The flow rate is set to 25 ml per minute. The diluted mRNA lipid nanoparticle suspension is collected into a sterile 500 ml PETG bottle. For the second run, the settings are changed on the syringe pump to the correct size and manufacturer (60 ml, BD Plastipak). The flow rate is set to 25 mL per minute. The diluted mRNA lipid nanoparticle suspension is collected into the same sterile 500 mL PETG bottle. The final volume will be approximately 370-380 mL.

Dialysis and Concentration of mRNA Lipid Nanoparticles by Tangential Flow Filtration.

For a 15 mg of mRNA in the encapsulation run, Vivaflow 50 cartridge can be used. For a 30 mg mRNA encapsulation run, 2 Vivaflow 50 cartridges can be attached in series. Optionally, the regenerated cellulose cartridges may first be rendered pyrogen-free. Optionally, this procedure may be started the day before the encapsulation.

Using a Minimate TFF system, two Vivaflow 50 cartridges are set up in series and tubing attached. 500 mL of pyrogen-free, nuclease-free water is loaded into the reservoir and run through cartridges at 20 psi pressure. 100 mL of 0.1 M NaOH/1.0 M NaCl is loaded into the reservoir and 50 mL run through the cartridges. The remaining 50 mL stand in the cartridges overnight. The next morning, the remaining 50 mL is run through the cartridges at 20 psi. More pyrogen-free, nuclease free water is loaded into the reservoir and 50 mL is run through the cartridges. Repeat this water wash two more times. Test an aliquot of the last water rinse for endotoxin to ensure that it is below detectable amounts.

Load the mRNA lipid nanoparticle suspension into the Minimate TFF reservoir. Concentrate the mixture while maintaining an overall pressure of 20-25 psi. The filtrate might elute at approximately 4 ml per minute to start, but could then slow down. Concentrate until the liquid level in the reservoir is at the 40 ml graduation. Diafilter the concentrated mRNA lipid nanoparticle suspension against 300 ml of pyrogen-free, nuclease-free 1×PBS. Keep the pressure of 20-25 psi. After diafiltration, resume concentration of the material to just below the 10 ml graduation mark on the reservoir. Collect the mRNA lipid nanoparticle suspension from the reservoir. It is possible to rinse the TFF system with additional 5 ml of 1×PBS and to collect this wash that contains diluted mRNA lipid nanoparticle suspension, but this wash can be collected separately from the concentrated mRNA lipid nanoparticle suspension. Store materials at 4° C. until analysis. Wash the TFF system with pyrogen-free, nuclease-free water, followed by 70% ethanol/water.

### Example 36

### Pharmacokinetics of Leptin Protein Expression Following Subcutaneous Dosing of Leptin mRNA in Cationic Lipid B or Cationic Lipid C to Lean C57BI/6, Diet-Induced Obese (DIO), or Leptin-Deficient Ob/Ob Mice

To determine if the amount of subcutaneous fat influences the amount of leptin protein expressed after subcutaneous delivery of leptin mRNA, three different mouse models were evaluated.

8 week old male lean C57BI/6 mice from Jackson Labs, 13 week old male diet-induced obese (DIO) mice from Taconic, and 16 week old male leptin-deficient ob/ob mice from Jackson Labs were housed four per cage with a normal light cycle (6:00-18:00). They were given the HPLC purified human leptin mRNA of the invention (SEQ ID NO: 4), in which the uridines were substituted with pseudouridine. The mRNA were packaged in Cationic Lipid B or Cationic Lipid C (N:P molar ratio=4:1) according to the formulation process of EXAMPLE 35. The formulation was diluted in injectable phosphate buffered saline (PBS). Mice were injected subcutaneously as described in EXAMPLE 8.

Groups of four mice each received 0.2, 0.6, or 2.0 milligrams per kilogram (mpk) of leptin mRNA formulated in Cationic Lipid B or Cationic Lipid C at 9 AM and blood was taken 6 hours later at 3 PM. Blood was also taken 24, 48, 72 and 96 hours after administration (9 AM each subsequent day for four days) for assessment of leptin protein levels.

Human leptin protein levels were measured in plasma according to the ELISA protocol in EXAMPLE 1. Leptin protein levels after subcutaneous administration of leptin mRNA in Cationic Lipid B are detailed in TABLE 52. Leptin protein levels after subcutaneous administration of leptin mRNA in Cationic Lipid C are detailed in TABLE 53.

The results with both Cationic Lipid formulations demonstrate greater leptin protein levels in the modestly obese DIO mouse model compared to the lean C57BI/6 mouse model. Leptin protein levels were even higher in the severely obese ob/ob mouse model compared to the modestly obese DIO mouse model. Thus, greater amounts of subcutaneous fat correlate with greater leptin protein expression after subcutaneous administration of leptin mRNA in either Cationic Lipid B or Cationic Lipid C.

TABLE 52
Leptin Protein Levels in Plasma After Subcutaneous Administration of Leptin mRNA in
Cationic Lipid B to Lean C57BI/6, DIO, and ob/ob Mice

| Time | | | | | | | | |
|---|---|---|---|---|---|---|---|---|
| point | C57BI/6 | C57BI/6 | C57BI/6 | DIO | DIO | ob/ob | ob/ob | ob/ob |
| (hours) | 0.2 mpk | 0.6 mpk | 2 mpk | 0.6 mpk | 2 mpk | 0.2 mpk | 0.6 mpk | 0.6 mpk |
| 6 | 3.5 | 2.3 | 9.4 | 12.6 | 20.3 | 6.4 | 6.9 | 21.4 |
| 24 | 1.2 | 2.4 | 8.6 | 10.6 | 22.0 | 15.6 | 19.8 | 55.5 |
| 48 | BLOQ | 0.5 | 3.1 | 4.3 | 13.6 | 6.2 | 9.0 | 24.8 |
| 72 | BLOQ | BLOQ | 1.6 | 1.1 | 6.9 | 2.0 | 3.9 | 12.1 |
| 96 | BLOQ | BLOQ | 0.8 | 0.9 | 3.1 | 0.8 | 0.9 | 6.1 |

Leptin protein levels expressed as ng/mL

TABLE 52
Leptin Protein Levels in Plasma After Subcutaneous Administration of Leptin mRNA in
Cationic Lipid C to Lean C57BI/6, DIO, and ob/ob Mice

| Time | | | | | | | | |
|---|---|---|---|---|---|---|---|---|
| point | C57BI/6 | C57BI/6 | C57BI/6 | DIO | DIO | ob/ob | ob/ob | ob/ob |
| (hours) | 0.2 mpk | 0.6 mpk | 2 mpk | 0.6 mpk | 2 mpk | 0.2 mpk | 0.6 mpk | 0.6 mpk |
| 6 | 1.4 | 2.2 | 4.0 | 7.3 | 10.9 | 3.5 | 6.3 | 10.9 |
| 24 | 0.6 | 2.0 | 7.2 | 8.1 | 14.8 | 7.3 | 21.8 | 39.6 |
| 48 | BLOQ | 0.5 | 1.9 | 2.4 | 7.0 | 1.8 | 5.7 | 15.0 |
| 72 | BLOQ | BLOQ | 0.9 | 0.7 | 2.3 | 0.8 | 2.0 | 7.5 |
| 96 | BLOQ | BLOQ | 4.0 | BLOQ | 0.8 | BLOQ | BLOQ | 2.3 |

Leptin protein levels expressed as ng/mL

### Example 37

### Pharmacokinetics of Repeated SC Treatment of Leptin mRNA Packaged with Cationic Lipid B and Cationic Lipid C Administered to Leptin-Deficient Ob/Ob Mice

The purpose of this EXAMPLE was to demonstrate that the repeated (weekly) treatment of leptin mRNA leads to consistent (stable) leptin level following each successive dose and that the in vivo efficacy is maintained throughout dosing period. HPLC purified human leptin mRNA (SEQ ID NO: 4) in which the uridines were substituted with pseudouridine was packaged in Cationic Lipid B and C and administered subcutaneously to a mouse model of genetic leptin deficiency.

The animals used were 10 week old male ob/ob mice from Jackson Labs. Animals were single housed with a normal light cycle (6:00-18:00). They were given the modified synthetic leptin mRNA of the invention (SEQ ID NO: 4) that had been HPLC purified, packaged in Cationic Lipid B or Cationic Lipid C at an N:P molar ratio of 4:1, and diluted in injectable phosphate buffered saline (PBS). The mice were restrained in the normal manner. The leptin mRNA was administered subcutaneously as shown in EXAMPLE 8.

Five mice received PBS (Group A). Five mice received the modified synthetic leptin mRNA at a dose of 0.6 mpk packaged in cationic lipid C (Group B). Five mice received the modified synthetic leptin mRNA at a dose of 0.2 mpk packaged in cationic lipid C (Group C). Five mice received the modified synthetic leptin mRNA at a dose of 0.06 mpk packaged in cationic lipid C (Group D). Five mice received the modified synthetic leptin mRNA at a dose of 0.6 mpk packaged in cationic lipid B (Group E). Five mice received the modified synthetic leptin mRNA at a dose of 0.2 mpk packaged in cationic lipid B (Group F). Five mice received the modified synthetic leptin mRNA at a dose of 0.06 mpk packaged in cationic lipid B (Group G). Five mice received the modified synthetic leptin mRNA at a dose of 0.02 mpk packaged in cationic lipid B (Group H).

On day 0, mice were weighed and sorted into groups according to their body weights, so that the average body weight per group was the same, and then dosed at 9:00 AM based on their grouping. All mice were also weighed at 9:00 AM on every subsequent day of the study.

Mice from each group were bled by tail nick on day 0 at 3:00 PM (6 hours post-dose), and then at 9:00 am on days 1, 7, 10, 15, 21, 22, 28, and 31. Plasma was isolated and human leptin protein levels were measured according to the ELISA protocol in EXAMPLE 1. CardioChek® PA (Cat no 730) and triglycerides (TG) test strips (Ref No. 1716), manufactured by Polymer Technology Systems, Inc., Indianapolis Ind. USA, were used to measure triglycerides. 15 μL of freshly collected EDTA-blood by mouse tail vein were applied to the meter and TG value was recorded within ˜2 minutes. AlphaTRAk® meter (Cat no 321250401) and glucose test strips (Ref 32109-46-50), manufactured by Abbott Laboratories, Inc., Abbott Park IL USA, were used to measure mice glucose level. Only one drop of whole blood from tail vein is need to read glucose from this meter within ˜20 seconds.

The TABLES below show the body weight, glucose levels and triglyceride levels from weekly (4 doses) treatment with HPLC purified human leptin mRNA of the invention (SEQ ID NO: 4) in which the uridines were substituted with pseudouridine and formulated in lipid nanoparticles containing either Cationic Lipid B or Cationic Lipid C.

TABLE 55
Body Weight (g)

| | PBS | Cationic Lipid C | | | Cationic Lipid B | | | |
|---|---|---|---|---|---|---|---|---|
| Day | N/A | 0.6 mpk | 0.2 mpk | 0.06 mpk | 0.6 mpk | 0.2 mpk | 0.06 mpk | 0.02 mpk |
| 0 | 39.4 ± 1.1 | 39.5 ± 1.0 | 39.5 ± 1.0 | 39.6 ± 1.0 | 39.7 ± 1.0 | 39.8 ± 1.0 | 39.9 ± 1.0 | 40.7 ± 1.3 |
| 7 | 42.4 ± 1.2 | 39.4 ± 0.8 | 40.8 ± 1.0 | 42.9 ± 0.8 | 36.6 ± 1.2 | 38.7 ± 1.2 | 41.5 ± 1.0 | 44.3 ± 1.2 |
| 10 | 42.9 ± 1.4 | 37.3 ± 0.7 | 10.3 ± 1.0 | 43.3 ± 0.9 | 33.8 ± 1.1 | 36.5 ± 1.3 | 40.7 ± 0.8 | 44.5 ± 1.2 |
| 14 | 43.9 ± 1.8 | 39.7 ± 0.7 | 42.6 ± 0.9 | 44.7 ± 1.0 | 36.7 ± 1.1 | 39.3 ± 1.2 | 43.7 ± 0.8 | 46.7 ± 1.2 |
| 15 | 44.2 ± 1.9 | 39.1 ± 0.7 | 42.5 ± 1.0 | 45.0 ± 1.2 | 35.7 ± 1.1 | 38.1 ± 1.4 | 43.2 ± 0.9 | 46.4 ± 1.1 |
| 21 | 44.6 ± 2.3 | 39.9 ± 0.7 | 45.0 ± 0.8 | 47.1 ± 1.2 | 36.7 ± 0.9 | 39.8 ± 1.5 | 44.7 ± 1.0 | 48.5 ± 1.2 |
| 22 | 44.4 ± 2.3 | 39.0 ± 0.7 | 43.7 ± 0.8 | 45.9 ± 1.0 | 35.3 ± 1.0 | 39.0 ± 1.5 | 43.3 ± 1.2 | 47.8 ± 1.2 |
| 28 | 44.4 ± 2.5 | 41.0 ± 0.9 | 45.4 ± 0.6 | 47.9 ± 1.0 | 38.0 ± 1.0 | 41.2 ± 1.7 | 45.1 ± 1.2 | 50.2 ± 1.4 |

TABLE 56
Gluclose (mg/dL)

| | PBS | Cationic Lipid C | | | Cationic Lipid B | | | |
|---|---|---|---|---|---|---|---|---|
| Day | N/A | 0.6 mpk | 0.2 mpk | 0.06 mpk | 0.6 mpk | 0.2 mpk | 0.06 mpk | 0.02 mpk |
| 10 | 309.2 ± 36.5 | 115.4 ± 4.0 | 128.8 ± 15.0 | 224.8 ± 47.6 | 109.6 ± 16.4 | 121.2 ± 21.5 | 114.8 ± 12.1 | 171.6 ± 26.7 |
| 15 | 293.6 ± 38.9 | 102.4 ± 7.6 | 121.2 ± 17.6 | 187.6 ± 33.2 | 70.0 ± 6.0 | 84.0 ± 5.1 | 124.8 ± 11.8 | 139.6 ± 19.3 |
| 21 | 335.0 ± 56.0 | 85.6 ± 8.5 | 147.2 ± 12.8 | 241.6 ± 32.0 | 76.8 ± 3.3 | 87.4 ± 3.5 | 103.2 ± 14.2 | 169.2 ± 24.9 |
| 22 | 363.6 ± 60.0 | 110.0 ± 9.1 | 135.0 ± 21.1 | 173.4 ± 48.8 | 86.4 ± 8.2 | 95.6 ± 10.9 | 130.8 ± 21.0 | 137.40 ± 13.9 |
| 28 | 335.0 ± 60.4 | 82.8 ± 3.9 | 129.6 ± 38.3 | 217.6 ± 53.3 | 80.2 ± 0.7 | 95.4 ± 10.7 | 89.0 ± 13.7 | 123.8 ± 11.6 |
| 31 | 290.6 ± 75.0 | 124.8 ± 11.5 | 109.8 ± 3.1 | 135.4 ± 27.4 | 110.8 ± 9.3 | 139.8 ± 13.6 | 127.8 ± 11.4 | 117.0 ± 11.0 |

TABLE 57
Triglycerides (mg/dL)

| | PBS | Cationic Lipid C | | | Cationic Lipid B | | | |
|---|---|---|---|---|---|---|---|---|
| Day | N/A | 0.6 mpk | 0.2 mpk | 0.06 mpk | 0.6 mpk | 0.2 mpk | 0.06 mpk | 0.02 mpk |
| 28 | 314.2 ± 72.4 | 138.2 ± 10.2 | 109.8 ± 11.0 | 213.2 ± 77.4 | 108.2 ± 6.8 | 120.0 ± 18.0 | 95.6 ± 10.9 | 118.6 ± 13.6 |
| 31 | 296.0 ± 68.6 | 70.8 ± 3.9 | 70.4 ± 1.9 | 125.0 ± 51.3 | 79.0 ± 4.4 | 69.4 ± 1.7 | 66.6 ± 1.3 | 75.6 ± 5.8 |

TABLE 58
Leptin (ng/mL)

| | PBS | Cationic Lipid C | | | Cationic Lipid B | | | |
|---|---|---|---|---|---|---|---|---|
| Day | N/A | 0.6 mpk | 0.2 mpk | 0.06 mpk | 0.6 mpk | 0.2 mpk | 0.06 mpk | 0.02 mpk |
| 1 | | 20.4 ± 2.3 | 6.3 ± 1.3 | 1.0 ± 0.1 | 48.5 ± 6.0 | 25.4 ± 5.2 | 5.4 ± 1.1 | 1.4 ± 0.4 |
| 8 | | 14.4 ± 2.6 | 3.6 ± 0.6 | 1.0 ± 0.0 | 26.0 ± 8.1 | 19.2 ± 5.0 | 4.5 ± 0.2 | 0.9 ± 0.1 |
| 15 | | 17.6 ± 2.6 | 3.6 ± 0.5 | 0.8 ± 0.1 | 15.8 ± 3.1 | 14.9 ± 1.1 | 3.9 ± 0.4 | 1.0 ± 0.1 |
| 22 | | 29.3 ± 11.4 | 4.0 ± 0.8 | 0.9 ± 0.1 | 49.4 ± 24.7 | 52.5 ± 22.5 | 2.9 ± 0.2 | 0.8 ± 0.1 |

TABLE 59
Change in Body Weight (g)

| | PBS | Cationic Lipid C | | | Cationic Lipid B | | | |
|---|---|---|---|---|---|---|---|---|
| Day | N/A | 0.6 mpk | 0.2 mpk | 0.06 mpk | 0.6 mpk | 0.2 mpk | 0.06 mpk | 0.02 mpk |
| 0 | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 | 0.0 ± 0.0 |
| 1 | 0.5 ± 0.2 | −1.0 ± 0.1 | −0.4 ± 0.2 | 0.1 ± 0.1 | −1.2 ± 0.1 | −0.8 ± 0.2 | −0.3 ± 0.1 | 0.0 ± 0.2 |
| 2 | 0.2 ± 0.5 | −2.2 ± 0.3 | −1.2 ± 0.5 | −0.2 ± 0.2 | −3.2 ± 0.2 | −2.6 ± 0.5 | −1.4 ± 0.1 | −0.1 ± 0.2 |
| 3 | 0.7 ± 0.4 | −2.5 ± 0.3 | −1.0 ± 0.5 | 0.6 ± 0.2 | −4.3 ± 0.3 | −3.2 ± 0.8 | −1.0 ± 0.2 | 0.7 ± 0.3 |
| 4 | 1.2 ± 0.5 | −2.7 ± 0.3 | −0.7 ± 0.6 | 1.1 ± 0.2 | −5.1 ± 0.4 | −3.4 ± 1.0 | −0.8 ± 0.3 | 1.1 ± 0.4 |
| 5 | 2.5 ± 0.5 | −1.8 ± 0.3 | 0.3 ± 0.5 | 2.3 ± 0.2 | −4.4 ± 0.5 | −2.4 ± 1.1 | 0.5 ± 0.3 | 2.7 ± 0.5 |
| 6 | 2.7 ± 0.6 | −1.3 ± 0.4 | 0.9 ± 0.6 | 2.8 ± 0.2 | −4.0 ± 0.4 | −2.0 ± 1.0 | 1.1 ± 0.4 | 2.9 ± 0.2 |
| 7 | 3.0 ± 0.6 | −0.1 ± 0.3 | 1.3 ± 0.5 | 3.3 ± 0.2 | −3.1 ± 0.4 | −1.0 ± 1.0 | 1.7 ± 0.2 | 3.6 ± 0.4 |
| 8 | 2.9 ± 0.6 | −1.0 ± 0.3 | 1.3 ± 0.6 | 3.4 ± 0.2 | −4.0 ± 0.4 | −1.8 ± 1.0 | 1.5 ± 0.2 | 3.2 ± 0.4 |
| 9 | 2.8 ± 0.6 | −2.0 ± 0.3 | 0.4 ± 0.7 | 2.9 ± 0.3 | −5.5 ± 0.4 | −3.0 ± 1.1 | 0.4 ± 0.4 | 3.0 ± 0.5 |
| 10 | 3.6 ± 0.7 | −2.2 ± 0.4 | 0.8 ± 0.8 | 3.7 ± 0.2 | −5.9 ± 0.4 | −3.2 ± 1.0 | 0.8 ± 0.3 | 3.8 ± 0.5 |
| 11 | 3.6 ± 0.8 | −1.9 ± 0.4 | 1.3 ± 0.9 | 3.7 ± 0.3 | −5.5 ± 0.5 | −3.0 ± 1.0 | 1.4 ± 0.4 | 4.3 ± 0.4 |
| 12 | 4.2 ± 0.8 | −1.5 ± 0.5 | 1.8 ± 0.9 | 4.3 ± 0.3 | −4.7 ± 0.6 | −2.1 ± 1.0 | 2.3 ± 0.3 | 4.8 ± 0.4 |
| 13 | 3.9 ± 0.9 | −0.9 ± 0.5 | 2.2 ± 0.8 | 4.4 ± 0.2 | −4.2 ± 0.6 | −1.7 ± 1.0 | 2.6 ± 0.3 | 4.9 ± 0.5 |
| 14 | 4.5 ± 0.9 | 0.2 ± 0.5 | 3.1 ± 0.8 | 5.2 ± 0.2 | −3.0 ± 0.7 | −0.5 ± 0.9 | 3.8 ± 0.3 | 6.0 ± 0.8 |
| 15 | 4.8 ± 1.0 | −0.4 ± 0.4 | 2.9 ± 0.9 | 5.4 ± 0.3 | −4.0 ± 0.7 | −1.6 ± 1.5 | 3.3 ± 0.2 | 5.7 ± 0.4 |
| 16 | 4.4 ± 1.1 | −1.5 ± 0.5 | 2.2 ± 0.9 | 5.1 ± 0.2 | −5.3 ± 0.7 | −2.1 ± 1.2 | 2.4 ± 0.3 | 5.4 ± 0.6 |
| 17 | 4.7 ± 1.2 | −1.8 ± 0.5 | 2.9 ± 1.0 | 5.8 ± 0.3 | −5.9 ± 0.7 | −2.3 ± 1.2 | 2.7 ± 0.3 | 6.2 ± 0.8 |
| 18 | 5.3 ± 1.2 | −1.6 ± 0.4 | 3.7 ± 1.1 | 6.5 ± 0.4 | −5.5 ± 0.7 | −1.9 ± 1.4 | 3.0 ± 0.3 | 6.9 ± 1.0 |
| 19 | 5.4 ± 1.2 | −1.0 ± 0.5 | 4.3 ± 1.1 | 6.9 ± 0.4 | −4.8 ± 0.7 | −1.4 ± 1.3 | 3.6 ± 0.4 | 7.2 ± 0.7 |
| 20 | 5.1 ± 1.3 | −0.4 ± 0.4 | 4.7 ± 1.0 | 7.1 ± 0.4 | −4.1 ± 0.7 | −1.0 ± 1.3 | 4.0 ± 0.4 | 7.2 ± 0.6 |
| 21 | 5.3 ± 1.5 | 0.4 ± 0.3 | 5.5 ± 1.0 | 7.5 ± 0.5 | −3.0 ± 0.7 | 0.0 ± 1.3 | 4.8 ± 0.4 | 7.8 ± 0.7 |
| 22 | 5.1 ± 1.4 | −0.5 ± 0.3 | 4.2 ± 1.0 | 6.3 ± 0.2 | −4.5 ± 0.7 | −0.8 ± 1.4 | 3.4 ± 0.7 | 7.1 ± 0.8 |
| 23 | 4.6 ± 1.5 | −1.7 ± 0.3 | 3.4 ± 1.0 | 5.9 ± 0.4 | −5.3 ± 0.8 | −1.8 ± 1.3 | 2.7 ± 0.7 | 6.9 ± 0.9 |
| 24 | 4.8 ± 1.6 | −1.9 ± 0.3 | 3.6 ± 1.1 | 6.3 ± 0.4 | −5.5 ± 0.7 | −1.8 ± 1.3 | 2.8 ± 0.6 | 7.0 ± 0.8 |
| 25 | 4.7 ± 1.8 | −1.1 ± 0.3 | 4.2 ± 1.2 | 6.8 ± 0.4 | −4.7 ± 0.8 | −0.9 ± 1.4 | 3.4 ± 0.5 | 7.9 ± 0.8 |
| 26 | 4.9 ± 1.8 | 0.0 ± 0.3 | 4.8 ± 1.2 | 7.8 ± 0.3 | −3.5 ± 0.8 | 0.0 ± 1.4 | 4.2 ± 0.5 | 8.7 ± 0.9 |
| 27 | 5.2 ± 1.6 | 0.7 ± 0.4 | 5.3 ± 1.1 | 7.9 ± 0.4 | −2.5 ± 0.8 | 0.7 ± 1.4 | 4.6 ± 0.5 | 8.9 ± 1.0 |
| 28 | 5.1 ± 1.6 | 1.5 ± 0.3 | 5.9 ± 1.0 | 8.3 ± 0.3 | −1.7 ± 0.8 | 1.4 ± 1.4 | 5.2 ± 0.3 | 9.4 ± 0.9 |

### Example 38

### Glucose and Triglyceride Data Following Subcutaneous Treatment of Leptin mRNA Packaged with Cationic Lipid D Administered to Leptin-Deficient Ob/Ob Mice

The purpose of this EXAMPLE was to demonstrate the effects of leptin mRNA on the kinetics (rate of decrease and return to baseline) of glucose and triglycerides (TG) after single subcutaneous treatment. HPLC purified human leptin mRNA (SEQ ID NO: 4) in which the uridines were substituted with pseudouridine, was packaged in Cationic Lipid D and administered subcutaneously to a mouse model of genetic leptin deficiency.

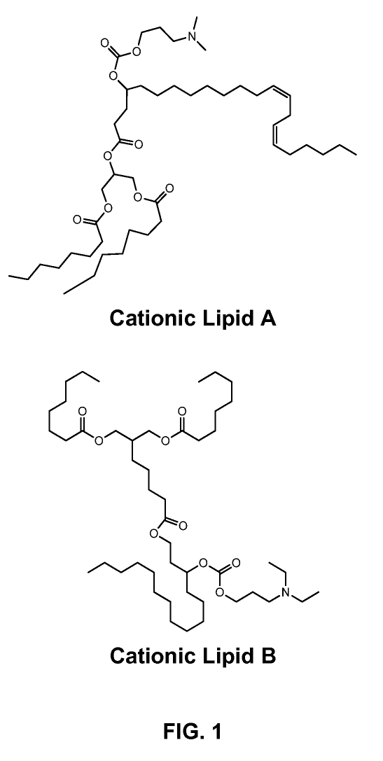

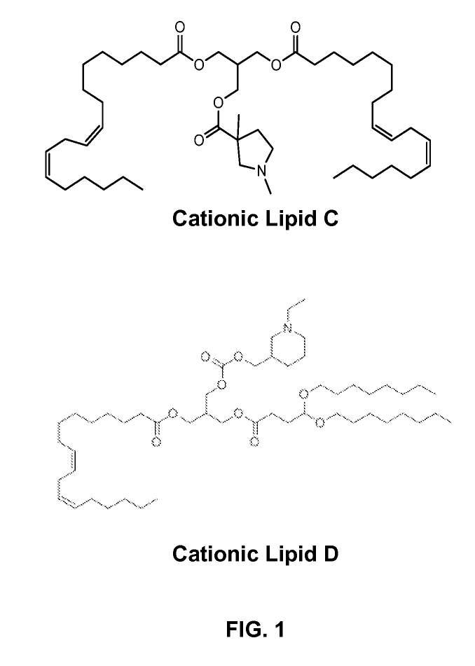

The structure of Cationic Lipid D is shown in FIG. 1.

The animals used were 10 week old male ob/ob mice from Jackson Labs. Animals were single housed with a normal light cycle (6:00-18:00). They were given the modified synthetic leptin mRNA of the invention (SEQ ID NO: 4) that had been HPLC purified, packaged in Cationic Lipid D at a N:P molar ratio of 4:1, diluted in injectable phosphate buffered saline (PBS) and injected subcutaneously as shown in EXAMPLE 8.

Four mice received PBS (Group A). Four mice received the modified synthetic leptin mRNA at a dose of 0.6 mpk packaged in cationic lipid D (Group B). Four mice received the modified synthetic leptin mRNA at a dose of 0.2 mpk packaged in cationic lipid D (Group C). Five mice received the modified synthetic leptin mRNA at a dose of 0.06 mpk packaged in Cationic Lipid D (Group D).

On day 0, mice were weighed and sorted into groups according to their fed glucose and triglyceride level, so that the average body weight per group was the same, and then dosed at 9:00 AM based on their grouping. All mice were also weighed at 9:00 AM on every subsequent day of the study.

Mice from each group were bled by tail nick on day 0 at 3:00 PM (6 hours post-dose), and then at 9:00 PM on days 1, 2, 3, 5, 6, 7 and 14. CardioChek® PA (Cat no 730) and triglycerides test strips (Ref No. 1716), were used to measure triglycerides, as described in EXAMPLE 37. 1AlphaTRAk® meter (Cat no 321250401) and glucose test strips (Ref 32109-46-50) were used to measure mice glucose levels, as also described in EXAMPLE 37.

TABLE 60
Glucose (mg/dL)

| | PBS | Cationic Lipid D | | |
|---|---|---|---|---|
| Day | A | B | C | D |
| 0 | 242.8 ± 20.1 | 249.5 ± 38.5 | 230.5 ± 13.8 | 227.2 ± 36.8 |
| 1 | 270.2 ± 19.2 | 197.0 ± 40.6 | 192.2 ± 38.9 | 213.8 ± 57.0 |
| 2 | 299.8 ± 16.1 | 128.5 ± 10.3 | 130.5 ± 26.7 | 160.5 ± 33.7 |
| 3 | 338.0 ± 31.4 | 118.0 ± 15.2 | 131.0 ± 13.0 | 177.2 ± 33.0 |
| 5 | 223.5 ± 49.8 | 90.2 ± 12.0 | 68.0 ± 4.3 | 127.2 ± 28.0 |
| 6 | 306.5 ± 32.1 | 87.8 ± 6.3 | 76.2 ± 3.2 | 168.8 ± 50.3 |
| 7 | 319.0 ± 30.4 | 83.5 ± 8.0 | 74.5 ± 9.7 | 170.0 ± 46.1 |
| 14 | 281.8 ± 26.3 | 160.2 ± 37.5 | 136.2 ± 19.1 | 212.5 ± 44.7 |

TABLE 61
Triglycerides (mg/dL)

| | PBS | Cationic Lipid D | | |
|---|---|---|---|---|
| Day | A | B | C | D |
| 0 | 249.8 ± 53.4 | 254.0 ± 44.7 | 261.2 ± 45.3 | 279.5 ± 49.7 |
| 1 | 315.0 ± 46.4 | 85.8 ± 6.5 | 112.0 ± 28.7 | 176.5 ± 52.8 |
| 2 | 375.2 ± 27.8 | 67.0 ± 6.2 | 66.2 ± 4.1 | 148.2 ± 27.1 |
| 3 | 456.2 ± 29.2 | 63.2 ± 2.7 | 83.2 ± 7.6 | 144.0 ± 23.4 |
| 5 | 396.8 ± 50.0 | 62.2 ± 1.9 | 63.8 ± 3.0 | 94.0 ± 12.1 |
| 7 | 377.8 ± 47.1 | 90.2 ± 6.3 | 96.2 ± 16.6 | 243.0 ± 82.8 |
| 14 | 292.5 ± 85.2 | 144.0 ± 40.6 | 260.5 ± 132.0 | 321.0 ± 67.9 |

### Example 39

### Toxicology Results for Cynomolqus Monkeys after Administration of Intravenous Administration of Leptin mRNA Packaged in Cationic Lipid B and Cationic Lipid C in Cynomolqus Monkeys

Cynomolgus monkeys received an intravenous administration of leptin mRNA packaged in lipid nanoparticles containing either Cationic Lipid B or Cationic Lipid C, as described in EXAMPLE 20. The brains were of the monkeys were examined for toxicological results either at 24 hours post-dose or at 72 hours post-dose.

Histological analyses of the spleens of some of the monkeys at 24 hours after intravenous administration showed increased cellularity in their periarterial lymphatic sheaths, with a shift towards a lymphohistiocytic population as compared to the mature lymphocytic population in control animals.

Histological analyses of some of the monkeys at 72 hours after intravenous administration of either of the lipid nanoparticles at the higher doses (e.g., at 1.5 mpk for Cationic Lipid B formulations or 1.25 mpk for Cationic Lipid C formulations) showed lesions and scattered foci of brain hemorrhage. The lesions were mostly restricted to the monkeys' brain white matter and were associated with mixed to predominantly neutrophilic inflammatory cell infiltration into the surrounding neuropil. The scattered focal hemorrhages were limited to the white matter.

Glial cell activation was observed in both the grey and white matter of the animals' cerebral cortex at 24 hours. Glial cell activation was measured immunohistochemically by increased GFAP expression (indicating activation in astrocytes) and increased Iba1 expression (indicating activation in microglial cells). Astrocyte activation was observed starting at 24 hours post-dose. The astrocyte activation was observed to be more pronounced at 72 hours post-dose in the animals showing histopathological lesions.

Other markers were also tested. Ki67 as measured immunohistochemically was increased in all high dose animals at the 24 and 72 hour time points. IL-1β was observed both by in situ hybridization and immunohistochemically in all high dose animals and was increased most in the monkeys that developed brain lesions. IL-1β mRNA and protein expression was observed at 24 hours and increases at 72 hours; at 24 hours mRNA expression appears to be present in astrocytes of grey and white matter. Loss of perivascular GFA-positive astrocytes was observed to occur at 72 hours in conjunction with inflammatory cell infiltrates and a loss of endothelial cell continuity. The presence of cleaved caspase 3 demonstrates apoptosis within the white matter of some of the affected animals at 72 hours after dosing. In situ hybridization results for human leptin RNA demonstrates the presence of leptin mRNA within glial cells and the endothelium of grey and white matter by 24 hours at all doses. At 72 hours, extensive localization was observed in the endothelium and glial cells of affected animals in regions of hemorrhage. Normal white matter endothelial cells expressed low levels of LDLR (as measured histochemically) which re-distributed after administration of the lipid nanoparticles to becomes upregulated in the affected animals observed. Immunohistochemistry results for C5b-9 (MAC) demonstrated complement deposition on endothelium of animals that received intravenous administration high dosages of at 72 hours. This complement deposition was observed to be more pronounced in affected animals and preceded the widespread the leptin signal (as detected by in situ hybridization) in the neuropil.

The scope of the invention is not intended to be limited to the above description, but rather is as set forth in the appended claims.

1. A formulation, comprising:
(a) a ribonucleic acid (RNA) polynucleotide,
(i) wherein the polynucleotide encodes a leptin protein, and
(ii) wherein the polynucleotide comprises a pseudouridine nucleotide; and
(b) a delivery agent.

2. The formulation of claim 1, wherein the encoded leptin protein is a human leptin protein.

3. The formulation of claim 1, wherein the ribonucleic acid polynucleotide comprises no uridine nucleotides.

4. The formulation of claim 1, wherein the ribonucleic acid polynucleotide comprises a coding region that has at least 78% sequence identity to a sequence selected from the group consisting SEQ ID NOS: 17-20.

5. The formulation of claim 1, wherein the ribonucleic acid polynucleotide comprises a coding region that has at least 90% sequence identity to a sequence selected from the group consisting SEQ ID NOS: 17-20.

6. The formulation of claim 1, wherein the ribonucleic acid polynucleotide comprises a sequence selected from the group consisting of SEQ ID NOS: 17-20.

7. The formulation of claim 1, wherein the ribonucleic acid polynucleotide comprises a sequence selected from the group consisting of SEQ ID NO: 4, SEQ ID NO: 6, SEQ ID NO: 8 and SEQ ID NO: 10.

8. The formulation of claim 1, wherein the formulation is a lipid nanoparticle (LNP).

9. The formulation of claim 1, wherein the delivery agent comprises a cationic lipid, a neutral lipid, a helper lipid and a stealth lipid.

10. The formulation of claim 9, wherein the molar ratio of cationic lipid to ribonucleic acid polynucleotide is between 3:1 and 8:1.

11. The formulation of claim 9, wherein the cationic lipid is selected from the group consisting of Cationic Lipid A, Cationic Lipid B, Cationic Lipid C and Cationic Lipid D.

12. The formulation of claim 9, wherein the neutral lipid is 1,2-distearoyl-sn-glycero-3-phosphocholine (DSPC).

13. The formulation of claim 9, wherein the helper lipid is cholesterol.

14. The formulation of claim 9, wherein the stealth lipid is a polyethylene glycol (PEG) lipid.

15. The formulation of claim 9, wherein the stealth lipid is S024.

16. The formulation of claim 1, wherein the delivery agent comprises a cationic lipid, cholesterol, a neutral lipid and a polyethylene glycol (PEG) lipid.

17-18. (canceled)

19. A method for treating a leptin deficiency, lipodystrophy or other condition where circulating leptin level is low in a subject, comprising the steps of:
(a) selecting a subject with a leptin deficiency, lipodystrophy or other condition where circulating leptin level is low;
(b) administering to the subject a formulation of claim 1;
wherein the administration of the formulation alleviates the symptoms of the subject's leptin deficiency, lipodystrophy or other condition where circulating leptin level is low.

20. The method of claim 19, wherein the formulation is administered in repeated doses.

21. The method of claim 19, wherein the formulation is administered intravenously.

22. The method of claim 19, wherein the formulation is administered subcutaneously.

23. The method of claim 19, wherein the formulation is administered intravenously, then administered subcutaneously.

24. The method of claim 19, wherein the formulation is administered at a dosage of at least 0.2 mg of ribonucleic acid polynucleotide/kg of the subject's body weight.

25. The method of claim 19, wherein the formulation is administered at dosage of at least 0.6 mg of ribonucleic acid polynucleotide/kg of the subject's body weight.

26. The method of claim 19, wherein the formulation is administered at an amount sufficient for a plasma leptin protein concentration of at least 1.4 ng/mL.

27. The method of claim 19, wherein the formulation is administered at an amount sufficient for a plasma leptin protein concentration of at least 2.8 ng/mL.

28. The method of claim 19, wherein the formulation is administered at an amount sufficient for a plasma leptin protein concentration of at least 10 ng/mL above the subject's plasma leptin protein concentration before the administration of the formulation.

29. The method of claim 19, wherein the formulation is administered at an amount sufficient for a plasma leptin protein concentration of at least 19 ng/mL.

30. The method of claim 19, wherein the formulation is administered at an amount sufficient for a plasma leptin protein concentration of at least 25 ng/mL.

31. The method of claim 19, wherein the formulation is administered at an amount sufficient for a plasma leptin protein concentration of at least 185 ng/mL.

32. The method of claim 19, wherein the formulation is administered at an amount sufficient for a plasma leptin protein concentration of at least 1300 ng/mL.

33. The method of claim 19, wherein the administration results in a decrease of plasma concentration of glucose by at least 30%.

34. The method of claim 19, wherein the administration results in a decrease of plasma concentration of triglycerides by at least 40%.

35. The method of claim 19, wherein the administration results in a stable body weight.

36. The method of claim 19, wherein the administration results in sustained weight loss.
